(19) United States
(12) Patent Application Publication
(10) Pub. No.: US 2010/0196349 A1
(43) Pub. Date: Aug. 5, 2010

(54) REELER DOMAIN CONTAINING PROTEIN

(75) Inventors: Christine Power, Thoiry (FR); Melanie Yorke-Smith, Geneva (CH); Richard Fagan, London (GB); Tania Rodrigues, London (GB); Dave Michalovich, London (GB)

Correspondence Address: SALIWANCHIK LLOYD & SALIWANCHIK;A PROFESSIONAL ASSOCIATION, PO Box 142950, GAINESVILLE, FL, 32614, US

(73) Assignee: ARES TRADING S.A., Aubonne (CH)

(21) Appl. No.: 12/095,593

(22) PCT Filed: Dec. 4, 2006
(86) PCT No.: PCT/GB2006/004517

§ 371 Date: Sep. 17, 2009

(30) Foreign Application Priority Data
Dec. 2, 2005 (GB) 0524648.3

(51) Int. Cl. A61K 39/395; C07K 14/00; C07H 21/00; C12N 15/74; C12N 5/10; C12Q 1/68; A61K 38/16; A61K 35/12; A61P 25/28

(52) U.S. Cl. 424/130.1; 530/350; 530/324; 536/23.1; 435/320.1; 435/325; 435/6; 514/12; 424/93.21

(57) ABSTRACT

This invention relates to protein INSP 171, herein identified as containing a Reeler domain, and to the use of this protein and the nucleic acid sequence from the encoding gene in the diagnosis, prevention and treatment of disease.

This invention relates to a protein, termed INSP171, herein identified as containing a Reeler domain, and to the use of this protein and nucleic acid sequences from the encoding gene in the diagnosis, prevention and treatment of disease.

All publications, patents and patent applications cited herein are incorporated in full by reference.

### BACKGROUND

The process of drug discovery is presently undergoing a fundamental revolution as the era of functional genomics comes of age. The term “functional genomics” applies to an approach utilising bioinformatics tools to ascribe function to protein sequences of interest. Such tools are becoming increasingly necessary as the speed of generation of sequence data is rapidly outpacing the ability of research laboratories to assign functions to these protein sequences.

As bioinformatics tools increase in potency and in accuracy, these tools are rapidly replacing the conventional techniques of biochemical characterisation. Indeed, the advanced bioinformatics tools used in identifying the present invention are now capable of outputting results in which a high degree of confidence can be placed.

Various institutions and commercial organisations are examining sequence data as they become available and significant discoveries are being made on an on-going basis. However, there remains a continuing need to identify and characterise further genes and the polypeptides that they encode, as targets for research and for drug discovery.

### Introduction to Secreted Proteins

The ability of cells to make and secrete extracellular proteins is central to many biological processes Enzymes, growth factors, extracellular matrix proteins and signalling molecules are all secreted by cells. This is through fusion of a secretory vesicle with the plasma membrane. In most cases, but not all, proteins are directed to the endoplasmic reticulum and into secretory vesicles by a signal peptide. Signal peptides are cis-acting sequences that affect the transport of polypeptide chains from the cytoplasm to a membrane bound compartment such as a secretory vesicle. Polypeptides that are targeted to the secretory vesicles are either secreted into the extracellular matrix or are retained in the plasma membrane. The polypeptides that are retained in the plasma membrane will have one or more transmembrane domains. Examples of signal peptide containing proteins that play a central role in the functioning of a cell are cytokines, hormones, extracellular matrix proteins, adhesion molecules, receptors, proteases, and growth and differentiation factors.

### Introduction to the Reeler Domain

Many eukaryotic cell-surface and secreted proteins show modular architectures with tandem arrays of distinct conserved domains. Specifically, a great diversity of domain architectures is seen in the surface receptors and extracellular matrix proteins of multicellular organisms.

Reeler domain containing proteins constitute a good example of this great diversity of domain architectures. Their architecture ranges from proteins containing solely a single Reeler region to combinations with domains as varied as a Kunitz-BPTI to Epidermal growth factor (EGF)-like.

The Reeler domain comprises ˜140 residues and evidence so far suggests it tends to be located at the N-terminus of a variety of secreted and surface proteins.

Reelin was amongst the first Reeler containing proteins identified, in this case there are additionally many EGF-like domains and BNR (Bacterial Neuraminidase Repeat) repeats (Genome Res. 1997 February; 7(2):157-64. The human reelin gene: isolation, sequencing, and mapping on chromosome 7. DeSilva U, D'Arcangelo G, Braden V V, Chen J, Miao G G, Curran T, Green E D). It constitutes a large extracellular matrix protein secreted by pioneer neurons that appears to function as an instructive signal in the regulation of cell positioning during neurodevelopment.

Members of the spondin family such as F-spondin also contain a Reeler domain in addition to thrombospondin type 1 (TSP-1) repeats. F-spondin is a matrix-attached adhesion molecule that promotes adhesion and outgrowth of hippocampal embryonic neurons and binds to a putative receptor(s) expressed on both hippocampal and sensory neurons. A Drosophila member of the spondin family, which is highly expressed in fat body and hemocytes (Fat-spondin, Genbank protein accession number AAD31715), also contains a Kunitz-BPTI domain besides the Reeler region and TSP-1 repeats.

Shirozu M et al., (Genomics 1996 Nov. 1; 37(3):273-80) identified another Reeler containing protein, a mouse stromal cell derived factor receptor 2 (SDR2). The mouse SDR, which was identified as a kidney-specific surface protein, additionally contains a Domon domain and a specific C-terminal six transmembrane ferric reductase domain. SDR2 is proposed to be important in regulating the metabolism of iron in the onset of neurodegenerative disorders.

Polypeptides of this nature are implicated in various diseases, including in particular, neurological disorders such as, for example, schizophrenia (see Impagnatiello et al., 1998, Proc Natl Acad Sci USA; 22; 95(26):15718-23), neurodegeneration, neurodevelopmental disease, stroke, brain/spinal cord injury, Alzheimer's disease, Parkinson's disease, motor neurone disease, neuromuscular disease, CNS inflammation, cerebellar degeneration, amylotrophic lateral sclerosis, head injury damage, and other neurological abnormalities, including Autosomal recessive lissencephaly with cerebellar hypoplasia (Hong et al., Nat Genet. 2000 September; 26(1):93-6) and autistic disorders (Persico et al., Mol Psychiatry. 2001 March; 6(2):150-9).

Reelin may be involved in schizophrenia, autism, bipolar disorder, major depression and in migration defects associated with temporal lobe epilepsy (Saez-Valero et al. J Neurosci Res. 2003 Apr. 1; 72(1):132-6. OMIM Accession Number 600514, NCBI gene ID 5649). Mutations of this gene are associated with autosomal recessive lissencephaly with cerebellar hypoplasia.

WO2003/039575 discloses methods of exposing a patient suffering from CNS disorders to a reagent that modulates the proliferation, migration, differentiation and survival of central nervous system cells via Reelin, Gas6 or Protein S signaling. Diseases or disorders of the nervous system is selected from the group consisting of neurodegenerative disorders, neural stem cell disorders, neural progenitor disorders, ischemic disorders, neurological kaumas, affective disorders, neuropsychiatric disorders, degenerative diseases of the retina, retinal injury/trauma and learning and memory disorders, Parkinson's disease and Parkinsonian disorders, Huntington's disease, Alzheimer's disease, Amyotrophic Lateral Sclerosis, spinal ischemia, ischemic stroke, spinal cord injury and cancer-related brain/spinal cord injury, schizophrenia and other psychoses, lissencephaly syndrome, depression, bipolar depression/disorder, anxiety syndromes/disorders, phobias, stress and related syndromes, cognitive function disorders, aggression, drug and alcohol abuse, obsessive compulsive behavior syndromes, seasonal mood disorder, borderline personality disorder, cerebral palsy, life style drug, multi-infarct dementia, Lewy body dementia, age related/geriatric dementia, epilepsy and injury related to epilepsy, temporal lobe epilepsy, spinal cord injury, brain injury, trauma related brain/spinal cord injury, anti-cancer treatment related brain/spinal cord tissue injury, infection and inflammation related brain/spinal cord injury, environmental toxin related brain/spinal cord injury, multiple sclerosis, autism, attention deficit disorders, narcolepsy and sleep disorders.

WO2004/078135 discloses a composition comprising an isolated F-spondin polypeptide that specifically bound to amyloid-13 precursor protein (APP) or the APP-like proteins (APLP 1 and APLP2) involved in Alzheimer's disease.

WO97/29189 discloses methods for utilizing an F-spondin polypeptide for treating spinal cord injuries and damage to peripheral nerves by promoting neural-cell adhesion and neurite extension, inhibiting tumor metastases and tumor angiogenesis, and stimulating wound repair. Antagonists are also disclosed which may be utilized to prevent malaria.

WO93/20196 discloses an isolated vertebrate nucleic acid molecule encoding F-spondin, a method of stimulating growth of a nerve cell comprising contacting the nerve cell with purified F-spondin, a method of regenerating nerve cells in a subject comprising administering to the subject purified F-spondin, and a pharmaceutical composition for stimulating nerve cell growth comprising a pharmaceutically acceptable carrier and purified F-spondin.

EP0897979 discloses SDR2 polypeptides and polynucleotides, methods for producing such polypeptides by recombinant techniques and methods for utilizing SDR2 polypeptides and polynucleotides in the design of protocols for the treatment of cancer, inflammation, autoimmunity, allergy, asthma, rheumatoid arthritis, CNS inflammation, cerebellar degeneration, Alzheimer's disease, Parkinson's disease, multiple sclerosis, amylotrophic lateral sclerosis, head injury damage, and other neurological abnormalities, septic shock, sepsis, stroke, osteoporosis, osteoarthritis, ischemia reperfusion injury, cardiovascular disease, kidney disease, liver disease, ischemic injury, myocardial infarction, hypotension, hypertension, AIDS, myelodysplastic syndromes and other hematologic abnormalities, aplastic anemia, male pattern baldness, and bacterial, fungal, protozoan and viral infections.

The identification of novel proteins containing Reeler domains will allow the development of new methods for the treatment and diagnosis of diseases and disorders in which these proteins are implicated. Accordingly, there remains a need for the identification of such proteins to enable new drugs to be developed for the treatment and prevention of human disease.

### THE INVENTION

The invention is based on the discovery that the INSP171 protein contains a Reeler domain.

In one embodiment of the first aspect of the invention, there is provided a polypeptide, which polypeptide:
i) comprises the amino acid sequence as recited in SEQ ID NO:2, SEQ ID NO:4, SEQ ID NO:6, SEQ ID NO:8 or SEQ ID NO:10;
ii) is a fragment thereof that contains a Reeler domain and/or has an antigenic determinant in common with the polypeptides of (i); or
iii) is a functional equivalent of (i) or (ii).

A polypeptide according to this aspect of the invention may consist of the amino acid sequence as recited in any one of SEQ ID NO:2, SEQ ID NO:4, SEQ ID NO:6, SEQ ID NO:8 or SEQ ID NO:10.

The polypeptide having the sequence recited in SEQ ID NO:2 is referred to hereafter as “the INSP171 mature polypeptide”. The polypeptide having the sequence recited in SEQ ID NO:4 is referred to hereafter as “the INSP171-SV1 mature polypeptide”. The polypeptide having the sequence recited in SEQ ID NO:6 is referred to hereafter as “INSP171-SV2 mature polypeptide”. The polypeptide having the sequence recited in SEQ ID NO:8 is referred to hereafter as “INSP171-SV3 mature polypeptide”. The polypeptide having the sequence recited in SEQ ID NO:10 is referred to hereafter INSP171-SV4 mature polypeptide”.

The polypeptide having the sequence recited in SEQ ID NO:12 is referred to hereafter as “INSP171 his tag mature polypeptide”. The polypeptide having the sequence recited in SEQ ID NO:14 is referred to hereafter as “INSP171-SV1 his tag mature polypeptide”. The polypeptide having the sequence recited in SEQ ID NO:16 is referred to hereafter as “INSP171-SV2 his tag mature polypeptide”. The polypeptide having the sequence recited in SEQ ID NO:18 is referred to hereafter as “INSP171-SV3 his tag mature polypeptide”. The polypeptide having the sequence recited in SEQ ID NO:20 is referred to hereafter as “INSP171-SV4 his tag mature polypeptide”. The polypeptide having the sequence recited in SEQ ID NO:22 is referred to hereafter as “INSP171 full length polypeptide”.

Two alternative translation start sites were predicted at amino acid positions 1 and 3 of the 327 residue sequence of SEQ ID NO:22. The polypeptide having the sequence recited in SEQ ID NO:24 is referred to hereafter as “INSP171-SV1 Full length polypeptide”. The polypeptide having the sequence recited in SEQ ID NO:26 is referred to hereafter as “INSP171-SV2 Full length polypeptide”. The polypeptide having the sequence recited in SEQ ID NO:28 is referred to hereafter as “INSP171-SV3 Full length polypeptide”. The polypeptide having the sequence recited in SEQ ID NO:30 is referred to hereafter as “INSP171-SV4 Full length polypeptide”.

The polypeptide having the sequence recited in SEQ ID NO:32 is referred to hereafter as “INSP171 his tag Full length polypeptide”. The polypeptide having the sequence recited in SEQ ID NO:34 is referred to hereafter as “INSP171-SV1 his tag Full length polypeptide”. The polypeptide having the sequence recited in SEQ ID NO:36 is referred to hereafter as “INSP171-SV2 his tag Full length polypeptide”. The polypeptide having the sequence recited in SEQ ID NO:38 is referred to hereafter as “INSP171-SV3 his tag Full length polypeptide”. The polypeptide having the sequence recited in SEQ ID NO:40 is referred to hereafter as “INSP171-SV4 his tag Full length polypeptide”.

Polypeptides containing any combination of the splice variants listed above are included in the first aspect of the invention.

Although the Applicant does not wish to be bound by this theory, it is postulated that the first 23 amino acids of the INSP171 polypeptide that starts from the methionine at position 1 of SEQ ID NO:22 form a signal peptide, as shown in the schematic representation below:

INSP171 Exon 1a

(SEQ ID NO: 41)

MRMQAALVGWACTTLCLASCSSAFSHGASTVACDDMQPKHIQAQPQHQDS
HHITIHTHRTSYAPGDKIPV

Similarly, it is postulated that the first 21 amino acids of the INSP171 polypeptide that starts from the methionine at position 3 of SEQ ID NO:22 form a signal peptide, as shown in the schematic representation below:

INSP171 Exon 1b

(SEQ ID NO: 42)

MQAALVGWACTTLCLASCSSAFSHGASTVACDDMQPKHIQAQPQHQDSHH
ITIHTHRTSYAPGDKIPV

The full length INSP171 polypeptide sequence without the postulated signal sequence is recited in SEQ ID NO:2. The polypeptide having the sequence recited in SEQ ID NO:2 is referred to hereafter as “the INSP171 mature polypeptide”.

Functional equivalents of the INSP171 polypeptides may include splice variants of the INSP171 polypeptides specifically exemplified herein.

Preferably, the “Reeler domain containing protein” may be a molecule containing a Reeler domain detected with an e-value lower than 0.1, 0.01, 0.001, 0.0001, 0.00001, 0.000001 or 0.0000001.

Preferably, a polypeptide according to the invention functions as a Reeler domain containing polypeptide. The term “Reeler domain” will be understood by the skilled person, who will readily be able to ascertain whether any given polypeptide falls within this definition. The Reeler domain comprises ˜140 residues and evidence so far suggests it tends to be located at the N-terminus of a variety of secreted and surface proteins.

By “functions as a Reeler domain containing polypeptide” we refer to polypeptides that comprise amino acid sequence or structural features that can be identified as conserved features within Reeler domain containing polypeptides, such that a biological activity is shared with other Reeler domain containing polypeptides.

The Reeler domain identified in INSP171 full length polypeptide spans from residues 32-157. The Reeler domain identified in the INSP171-SV1 polypeptide spans from residues 32-144. The Reeler domains identified in the INSP171-SV2, INSP171-SV3 and INSP171-SV4 polypeptides span from residues 32-69 in these sequences.

A complete Reeler domain has been predicted in INSP171 as well as two N-glycosylated Asparagine residues. Glycosylated forms of this polypeptide form one aspect of the invention. INSP171-SV1 contains most of the Reeler domain however the last predicted N-Glycosylated Asparagine is absent. Glycosylated forms of these polypeptides form one aspect of the invention, particularly at the asparagine residues indicated in FIG. 3.

In INSP171-SV2, INSP171-SV3 and INSP171-SV4 only incomplete Reeler domains are present and they lack any predicted N-glycosylated Asparagines.

This aspect of the invention also includes fusion proteins that incorporate polypeptide fragments and variants of these polypeptide fragments as defined above.

The polypeptides of the first aspect of the invention may further comprise a histidine tag. Preferably the histidine tag is found at the C-terminal of the polypeptide. Preferably the histidine tag comprises 1-10 histidine residues (e.g. 1, 2, 3, 4, 5, 6, 7, 8, 9 or 10 residues). More preferably the histidine tag comprises 6 histidine residues.

An “antigenic determinant” of the present invention may be a part of a polypeptide of the present invention, which binds to an antibody-combining site or to a T-cell receptor (TCR). Alternatively, an “antigenic determinant” may be a site on the surface of a polypeptide of the present invention to which a single antibody molecule binds. Generally an antigen has several or many different antigenic determinants and reacts with antibodies of many different specificities. Preferably, the antibody is immunospecific to a polypeptide of the invention. Preferably, the antibody is immunospecific to a polypeptide of the invention, which is not part of a fusion protein. Preferably, the antibody is immunospecific to INSP171 or a fragment thereof. Antigenic determinants usually consist of chemically active surface groupings of molecules, such as amino acids or sugar side chains, and can have specific three dimensional structural characteristics, as well as specific charge characteristics. Preferably, the “antigenic determinant” refers to a particular chemical group on a polypeptide of the present invention that is antigenic, i.e. that elicit a specific immune response.

In a second aspect, the invention provides a purified nucleic acid molecule which encodes a polypeptide of the first aspect of the invention.

The term “purified nucleic acid molecule” preferably refers to a nucleic acid molecule of the invention that (1) has been separated from at least about 50 percent of proteins, lipids, carbohydrates, or other materials with which it is naturally found when total nucleic acid is isolated from the source cells, (2) is not linked to all or a portion of a polynucleotide to which the “purified nucleic acid molecule” is linked in nature, (3) is operably linked to a polynucleotide which it is not linked to in nature, or (4) does not occur in nature as part of a larger polynucleotide sequence. Preferably, the isolated nucleic acid molecule of the present invention is substantially free from any other contaminating nucleic acid molecule(s) or other contaminants that are found in its natural environment that would interfere with its use in polypeptide production or its therapeutic, diagnostic, prophylactic or research use. In a preferred embodiment, genomic DNA are specifically excluded from the scope of the invention. Preferably, genomic DNA larger than 10 kbp (kilo base pairs), 50 kbp, 100 kbp, 150 kbp, 200 kbp, 250 kbp or 300 kbp are specifically excluded from the scope of the invention. Preferably, the “purified nucleic acid molecule” consists of cDNA only.

Preferably, the purified nucleic acid molecule comprises the nucleic acid sequence as recited in SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, and/or SEQ ID NO:39, or is a redundant equivalent or fragment of any one of these sequences.

Preferably, the purified nucleic acid molecule consists of the nucleic acid sequence as recited in SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, and/or SEQ ID NO:39.

In a third aspect, the invention provides a purified nucleic acid molecule which hybridizes under high stringency conditions with a nucleic acid molecule of the second aspect of the invention. High stringency hybridisation conditions are defined as overnight incubation at 42° C. in a solution comprising 50% formamide, 5×SSC (150 mM NaCl, 15 mM trisodium citrate), 50 mM sodium phosphate (pH7.6), 5×Denhardts solution, 10% dextran sulphate, and 20 microgram/ml denatured, sheared salmon sperm DNA, followed by washing the filters in 0.1×SSC at approximately 65° C.

In a fourth aspect, the invention provides a vector, such as an expression vector, that contains a nucleic acid molecule of the second or third aspect of the invention.

In a fifth aspect, the invention provides a host cell transformed with a vector of the fourth aspect of the invention.

In a sixth aspect, the invention provides a ligand which binds specifically to, and which preferably inhibits the activity of a polypeptide of the first aspect of the invention.

In a seventh aspect, the invention provides a compound that is effective to alter the expression of a natural gene which encodes a polypeptide of the first aspect of the invention or to regulate the activity of a polypeptide of the first aspect of the invention.

Such compounds may be identified using the assays and screening methods disclosed herein.

A compound of the seventh aspect of the invention may either increase (agonize) or decrease (antagonize) the level of expression of the gene or the activity of the polypeptide.

Importantly, the identification of the domain organisation and function of the INSP171 polypeptide allows for the design of screening methods capable of identifying compounds that are effective in the treatment and/or diagnosis of disease. Ligands and compounds according to the sixth and seventh aspects of the invention may be identified using such methods. These methods are included as aspects of the present invention. Using these methods, it will now be possible to identify inhibitors or antagonists of INSP171, such as, for example, monoclonal antibodies, which may be of use in modulating INSP171 activity in vivo in clinical applications. Such compounds are likely to be useful in counteracting the activity of the INSP171 polypeptides.

Another aspect of this invention resides in the use of an INSP171 gene or polypeptide as a target for the screening of candidate drug modulators, particularly candidate drugs active against Reeler domain containing protein related disorders.

A further aspect of this invention resides in methods of screening of compounds for therapy of Reeler domain containing protein related disorders, comprising determining the ability of a compound to bind to an INSP171 gene or polypeptide, or a fragment thereof.

A further aspect of this invention resides in methods of screening of compounds for therapy of Reeler domain containing protein related disorders, comprising testing for modulation of the activity of an INSP171 gene or polypeptide, or a fragment thereof.

In an eighth aspect, the invention provides a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, for use in therapy or diagnosis of diseases in which polypeptides containing Reeler domains are implicated. Such diseases include, but are not limited to neoplasm, cancer, brain tumour, glioma, bone tumor, lung tumor, breast tumour, prostate tumour, colon tumour, hepatocellular carcinoma or liver tumour, endometrial carcinoma, hemangioma, myeloproliferative disorder, leukemia, hematological disease, neutropenia, thrombocytopenia, angiogenesis disorders, dermatological disease, aging, wounds, burns, fibrosis, cardiovascular disease, restenosis, heart disease, peripheral vascular disease, coronary artery disease, oedema, thromboembolism, dysmenorrhea, endometriosis, pre-eclampsia, lung disease, COPD, asthma bone disease, renal disease, glomerulonephritis, liver disease, Crohn's disease, gastritis, ulcerative colitis, ulcer, immune disorder, autoimmune disease, arthritis, rheumatoid arthritis, psoriasis, epidermolysis bullosa, systemic lupus erythematosus, ankylosing spondylitis, Lyme disease, multiple sclerosis, neurodegeneration, stroke, brain/spinal cord injury, Alzheimer's disease, Parkinson's disease, motor neurone disease, neuromuscular disease, CNS inflammation, cerebellar degeneration, amylotrophic lateral sclerosis, head injury damage, and other neurological abnormalities, HIV, AIDS, cytomegalovirus infection and fungal infection.

Particularly, polypeptides of this nature are implicated in neurological disorders such as, for example, schizophrenia, neurodegeneration, neurodevelopmental disease, stroke, brain/spinal cord injury, Alzheimer's disease, Parkinson's disease, motor neurone disease, neuromuscular disease, CNS inflammation, cerebellar degeneration, amylotrophic lateral sclerosis, head injury damage, and other neurological abnormalities, including Autosomal recessive lissencephaly with cerebellar hypoplasia and autistic disorders.

Polypeptides containing the Reeler domain have been implicated in various diseases, including in particular, neurological disorders such as, for example, schizophrenia (see Impagnatiello et al., 1998, Proc Natl Acad Sci USA; 22; 95(26):15718-23), neurodegeneration, neurodevelopmental disease, stroke, brain/spinal cord injury, Alzheimer's disease, Parkinson's disease, motor neurone disease, neuromuscular disease, CNS inflammation, cerebellar degeneration, amylotrophic lateral sclerosis, head injury damage, and other neurological abnormalities, including Autosomal recessive lissencephaly with cerebellar hypoplasia (Hong et al., Nat Genet. 2000 September; 26(1):93-6) and autistic disorders (Persico et al., Mol Psychiatry. 2001 March; 6(2):150-9).

Reelin itself may be involved in schizophrenia, autism, bipolar disorder, major depression and in migration defects associated with temporal lobe epilepsy (Saez-Valero et al. J Neurosci Res. 2003 Apr. 1; 72(1):132-6. OMIM Accession Number 600514, NCBI gene ID 5649). Mutations of this gene are associated with autosomal recessive lissencephaly with cerebellar hypoplasia.

Cloning data for INSP171 and splice variants thereof as well as gene expression data for an homologous sequence to INSP171 (EST information for UniGene Cluster Hs.170876 corresponding to SDR2) indicate that INSP171 is expressed in hepatocellular carcinoma or liver tumour, and in endometrial carcinoma.

These moieties of the first, second, third, fourth, fifth, sixth or seventh aspect of the invention may also be used in the manufacture of a medicament for the treatment of such diseases.

This eighth aspect of the invention also provides a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, for use in therapy or diagnosis of diseases in which polypeptides containing Reeler domains are implicated. Such diseases include those listed above.

These moieties of the first, second, third, fourth, fifth, sixth or seventh aspect of the invention may also be used in the manufacture of a medicament for the treatment of such diseases.

In a ninth aspect, the invention provides a method of diagnosing a disease in a patient, comprising assessing the level of expression of a natural gene encoding a polypeptide of the first aspect of the invention or the activity of a polypeptide of the first aspect of the invention in tissue from said patient and comparing said level of expression or activity to a control level, wherein a level that is different to said control level is indicative of disease. Such a method will preferably be carried out in vitro. Similar methods may be used for monitoring the therapeutic treatment of disease in a patient, wherein altering the level of expression or activity of a polypeptide or nucleic acid molecule over the period of time towards a control level is indicative of regression of disease.

In a ninth aspect, the invention provides a method of diagnosing a disease in a patient, comprising assessing the level of expression of a natural gene encoding a polypeptide of the first aspect of the invention or the activity of a polypeptide of the first aspect of the invention in tissue from said patient and comparing said level of expression or activity to a control level, wherein a level that is different to said control level is indicative of disease. Such a method will preferably be carried out in vitro. Similar methods may be used for monitoring the therapeutic treatment of disease in a patient, wherein altering the level of expression or activity of a polypeptide or nucleic acid molecule over the period of time towards a control level is indicative of regression of disease.

A preferred method for detecting polypeptides of the first aspect of the invention comprises the steps of (a) contacting a ligand, such as an antibody, of the sixth aspect of the invention with a biological sample under conditions suitable for the formation of a ligand-polypeptide complex; and (b) detecting said complex.

A number of different such methods according to the ninth aspect of the invention exist, as the skilled reader will be aware, such as methods of nucleic acid hybridization with short probes, point mutation analysis, polymerase chain reaction (PCR) amplification and methods using antibodies to detect aberrant protein levels. Similar methods may be used on a short or long term basis to allow therapeutic treatment of a disease to be monitored in a patient. The invention also provides kits that are useful in these methods for diagnosing disease.

Preferably, the disease diagnosed by a method of the ninth aspect of the invention is a disease in which polypeptides containing Reeler domains are implicated, as described above.

In a tenth aspect, the invention provides for the use of the polypeptide of the first aspect of the invention as a polypeptide that contains a Reeler domain.

In an eleventh aspect, the invention provides a pharmaceutical composition comprising a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, in conjunction with a pharmaceutically-acceptable carrier.

In a twelfth aspect, the present invention provides a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, for use in the manufacture of a medicament for the diagnosis or treatment of a disease in which Reeler domain containing polypeptides are implicated. Such diseases include those described above in connection with the eighth aspect of the invention and include, but are not limited to neoplasm, cancer, brain tumour, glioma, bone tumor, lung tumor, breast tumour, prostate tumour, colon tumour, hepatocellular carcinoma or liver tumour, endometrial carcinoma, hemangioma, myeloproliferative disorder, leukemia, hematological disease, neutropenia, thrombocytopenia, angiogenesis disorders, dermatological disease, aging, wounds, burns, fibrosis, cardiovascular disease, restenosis, heart disease, peripheral vascular disease, coronary artery disease, oedema, thromboembolism, dysmenorrhea, endometriosis, pre-eclampsia, lung disease, COPD, asthma bone disease, renal disease, glomerulonephritis, liver disease, Crohn's disease, gastritis, ulcerative colitis, ulcer, immune disorder, autoimmune disease, arthritis, rheumatoid arthritis, psoriasis, epidermolysis bullosa, systemic lupus erythematosus, ankylosing spondylitis, Lyme disease, multiple sclerosis, neurodegeneration, stroke, brain/spinal cord injury, Alzheimer's disease, Parkinson's disease, motor neurone disease, neuromuscular disease, CNS inflammation, cerebellar degeneration, amylotrophic lateral sclerosis, head injury damage, and other neurological abnormalities, HIV, AIDS, cytomegalovirus infection and fungal infection.

In a thirteenth aspect, the invention provides a method of treating a disease in a patient comprising administering to the patient a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention.

For diseases in which the expression of a natural gene encoding a polypeptide of the first aspect of the invention, or in which the activity of a polypeptide of the first aspect of the invention, is lower in a diseased patient when compared to the level of expression or activity in a healthy patient, the polypeptide, nucleic acid molecule, vector, host cell, ligand or compound administered to the patient should be an agonist. Conversely, for diseases in which the expression of the natural gene or activity of the polypeptide is higher in a diseased patient when compared to the level of expression or activity in a healthy patient, the polypeptide, nucleic acid molecule, vector, host cell, ligand or compound administered to the patient should be an antagonist. Examples of such antagonists include antisense nucleic acid molecules, ribozymes and ligands, such as antibodies.

More preferably, the disease diagnosed by a method of the ninth aspect of the invention is a disease in which Reeler domain containing polypeptides are implicated, as described above.

The INSP171 polypeptides are Reeler domain containing proteins and thus have roles in many disease states. Antagonists of the INSP171 polypeptides are of particular interest as they provide a way of modulating these disease states.

In a fourteenth aspect, the invention provides transgenic or knockout non-human animals that have been transformed to express higher, lower or absent levels of a polypeptide of the first aspect of the invention. Such transgenic animals are very useful models for the study of disease and may also be used in screening regimes for the identification of compounds that are effective in the treatment or diagnosis of such a disease.

Preferably, the disease diagnosed by a method of the ninth aspect of the invention is a disease in which Reeler domain containing polypeptides are implicated, as described above.

As used herein, “functional equivalent” refers to a protein or nucleic acid molecule that possesses functional or structural characteristics that are substantially similar to a polypeptide or nucleic acid molecule of the present invention. A functional equivalent of a protein may contain modifications depending on the necessity of such modifications for the performance of a specific function. The term “functional equivalent” is intended to include the fragments, mutants, hybrids, variants, analogs, or chemical derivatives of a molecule.

Preferably, the “functional equivalent” may be a protein or nucleic acid molecule that exhibits any one or more of the functional activities of the polypeptides of the present invention.

Preferably, the “functional equivalent” may be a protein or nucleic acid molecule that displays substantially similar activity compared with INSP171 or fragments thereof in a suitable assay for the measurement of biological activity or function. Preferably, the “functional equivalent” may be a protein or nucleic acid molecule that displays identical or higher activity compared with INSP171 or fragments thereof in a suitable assay for the measurement of biological activity or function. Preferably, the “functional equivalent” may be a protein or nucleic acid molecule that displays 50%, 60%, 70%, 80%, 90%, 95%, 98%, 99%, 100% or more activity compared with INSP171 or fragments thereof in a suitable assay for the measurement of biological activity or function.

Preferably, the “functional equivalent” may be a protein or polypeptide capable of exhibiting a substantially similar in vivo or in vitro activity as the polypeptides of the invention. Preferably, the “functional equivalent” may be a protein or polypeptide capable of interacting with other cellular or extracellular molecules in a manner substantially similar to the way in which the corresponding portion of the polypeptides of the invention would. For example, a “functional equivalent” would be able, in an immunoassay, to diminish the binding of an antibody to the corresponding peptide (i.e., the peptide the amino acid sequence of which was modified to achieve the “functional equivalent”) of the polypeptide of the invention, or to the polypeptide of the invention itself, where the antibody was raised against the corresponding peptide of the polypeptide of the invention. An equimolar concentration of the functional equivalent will diminish the aforesaid binding of the corresponding peptide by at least about 5%, preferably between about 5% and 10%, more preferably between about 10% and 25%, even more preferably between about 25% and 50%, and most preferably between about 40% and 50%.

For example, functional equivalents can be fully functional or can lack function in one or more activities. Thus, in the present invention, variations can affect the function, for example, of the activities of the polypeptide that reflect its possession of a Reeler domain.

A summary of standard techniques and procedures which may be employed in order to utilise the invention is given below. It will be understood that this invention is not limited to the particular methodology, protocols, cell lines, vectors and reagents described. It is also to be understood that the terminology used herein is for the purpose of describing particular embodiments only and it is not intended that this terminology should limit the scope of the present invention. The extent of the invention is limited only by the terms of the appended claims.

Standard abbreviations for nucleotides and amino acids are used in this specification.

The practice of the present invention will employ, unless otherwise indicated, conventional techniques of molecular biology, microbiology, recombinant DNA technology and immunology, which are within the skill of those working in the art.

Such techniques are explained fully in the literature. Examples of particularly suitable texts for consultation include the following: Sambrook Molecular Cloning; A Laboratory Manual, Second Edition (1989); DNA Cloning, Volumes I and II (D. N Glover ed. 1985); Oligonucleotide Synthesis (M. J. Gait ed. 1984); Nucleic Acid Hybridization (B. D. Hames & S. J. Higgins eds. 1984); Transcription and Translation (B. D. Hames & S. J. Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. 1987, Cold Spring Harbor Laboratory); Immunochemical Methods in Cell and Molecular Biology (Mayer and Walker, eds. 1987, Academic Press, London); Scopes, (1987) Protein Purification: Principles and Practice, Second Edition (Springer Verlag, N.Y.); and Handbook of Experimental Immunology, Volumes I-IV (D. M. Weir and C. C. Blackwell eds. 1986).

As used herein, the term “polypeptide” includes any peptide or protein comprising two or more amino acids joined to each other by peptide bonds or modified peptide bonds, i.e. peptide isosteres. This term refers both to short chains (peptides and oligopeptides) and to longer chains (proteins).

The polypeptide of the present invention may be in the form of a mature protein or may be a pre-, pro- or prepro-protein that can be activated by cleavage of the pre-, pro- or prepro-portion to produce an active mature polypeptide. In such polypeptides, the pre-, pro- or prepro-sequence may be a leader or secretory sequence or may be a sequence that is employed for purification of the mature polypeptide sequence.

The polypeptide of the first aspect of the invention may form part of a fusion protein. For example, it is often advantageous to include one or more additional amino acid sequences which may contain secretory or leader sequences, pro-sequences, sequences which aid in purification, or sequences that confer higher protein stability, for example during recombinant production. Alternatively or additionally, the mature polypeptide may be fused with another compound, such as a compound to increase the half-life of the polypeptide (for example, polyethylene glycol).

In a further preferred embodiment, a polypeptide of the invention, that may comprise a sequence having at least 85% of homology with INSP171, INSP171-SV1, INSP171-SV2, INSP171-SV3 and/or INSP171-SV4, is a fusion protein.

These fusion proteins can be obtained by cloning a polynucleotide encoding a polypeptide comprising a sequence having at least 85% of homology with INSP171, INSP171-SV1, INSP171-SV2, INSP171-SV3 and/or INSP171-SV4 in frame to the coding sequences for a heterologous protein sequence.

The term “heterologous”, when used herein, is intended to designate any polypeptide other than a human INSP171, INSP171-SV1, INSP171-SV2, INSP171-SV3 and/or INSP171-SV4 polypeptide.

Example of heterologous sequences, that can be comprised in the soluble fusion proteins either at N- or at C-terminus, are the following: extracellular domains of membrane-bound protein, immunoglobulin constant regions (Fc region), multimerization domains, domains of extracellular proteins, signal sequences, export sequences, or sequences allowing purification by affinity chromatography.

Many of these heterologous sequences are commercially available in expression plasmids since these sequences are commonly included in the fusion proteins in order to provide additional properties without significantly impairing the specific biological activity of the protein fused to them (Terpe K, Appl Microbiol Biotechnol, 60: 523-33, 2003). Examples of such additional properties are a longer lasting half-life in body fluids, the extracellular localization, or an easier purification procedure as allowed by the a stretch of Histidines forming the so-called “histidine tag” (Gentz et al., Proc Natl Acad Sci USA, 86: 821-4, 1989) or by the “HA” tag, an epitope derived from the influenza hemagglutinin protein (Wilson et al., Cell, 37: 767-78, 1994). If needed, the heterologous sequence can be eliminated by a proteolytic cleavage, for example by inserting a proteolytic cleavage site between the protein and the heterologous sequence, and exposing the purified fusion protein to the appropriate protease. These features are of particular importance for the fusion proteins since they facilitate their production and use in the preparation of pharmaceutical compositions. For example, the protein used in the examples (INSP171, INSP171-SV1, INSP171-SV2, INSP171-SV3 and/or INSP171-SV4) can be purified by means of a hexa-histidine peptide fused at the C-terminus of INSP171, INSP171-SV1, INSP171-SV2, INSP171-SV3 and/or INSP171-SV4. When the fusion protein comprises an immunoglobulin region, the fusion may be direct, or via a short linker peptide which can be as short as 1 to 3 amino acid residues in length or longer, for example, 13 amino acid residues in length. Said linker may be a tripeptide of the sequence E-F-M (Glu-Phe-Met), for example, or a 13-amino acid linker sequence comprising Glu-Phe-Gly-Ala-Gly-Leu-Val-Leu-Gly-Gly-Gln-Phe-Met (SEQ ID NO: 43) introduced between the sequence of the substances of the invention and the immunoglobulin sequence. The resulting fusion protein has improved properties, such as an extended residence time in body fluids (half-life), increased specific activity, increased expression level, or the purification of the fusion protein is facilitated.

In a preferred embodiment, the protein is fused to the constant region of an Ig molecule. Preferably, it is fused to heavy chain regions, like the CH2 and CH3 domains of human IgG1, for example. Other isoforms of Ig molecules are also suitable for the generation of fusion proteins according to the present invention, such as isoforms IgG₂ or IgG₄, or other Ig classes, like IgM or IgA, for example. Fusion proteins may be monomeric or multimeric, hetero- or homomultimeric.

In a further preferred embodiment, the functional derivative comprises at least one moiety attached to one or more functional groups, which occur as one or more side chains on the amino acid residues. Preferably, the moiety is a polyethylene (PEG) moiety. PEGylation may be carried out by known methods, such as the ones described in WO99/55377, for example.

Polypeptides may contain amino acids other than the 20 gene-encoded amino acids, modified either by natural processes, such as by post-translational processing or by chemical modification techniques which are well known in the art. Among the known modifications which may commonly be present in polypeptides of the present invention are glycosylation, lipid attachment, sulphation, gamma-carboxylation, for instance of glutamic acid residues, hydroxylation and ADP-ribosylation. Other potential modifications include acetylation, aceylation, imidation, covalent attachment of flavin, covalent attachment of a haeme moiety, covalent attachment of a nucleotide or nucleotide derivative, covalent attachment of a lipid derivative, covalent attachment of phosphatidylinositol, cross-linking, cyclization, disulphide bond formation, demethylation, formation of covalent cross-links, formation of cysteine, formation of pyroglutamate, formylation, GPI anchor formation, iodination, methylation, myristoylation, oxidation, proteolytic processing, phosphorylation, prenylation, racemization, selenoylation, transfer-RNA mediated addition of amino acids to proteins such as arginylation, and ubiquitination.

Modifications can occur anywhere in a polypeptide, including the peptide backbone, the amino acid side-chains and the amino or carboxyl termini. In fact, blockage of the amino or carboxyl terminus in a polypeptide, or both, by a covalent modification is common in naturally-occurring and synthetic polypeptides and such modifications may be present in polypeptides of the present invention.

The modifications that occur in a polypeptide often will be a function of how the polypeptide is made. For polypeptides that are made recombinantly, the nature and extent of the modifications in large part will be determined by the post-translational modification capacity of the particular host cell and the modification signals that are present in the amino acid sequence of the polypeptide in question. For instance, glycosylation patterns vary between different types of host cell.

The polypeptides of the present invention can be prepared in any suitable manner. Such polypeptides include isolated naturally-occurring polypeptides (for example purified from cell culture), recombinantly-produced polypeptides (including fusion proteins), synthetically-produced polypeptides or polypeptides that are produced by a combination of these methods.

The functionally-equivalent polypeptides of the first aspect of the invention may be polypeptides that are homologous to the INSP171 polypeptides. Two polypeptides are said to be “homologous”, as the term is used herein, if the sequence of one of the polypeptides has a high enough degree of identity or similarity to the sequence of the other polypeptide. “Identity” indicates that at any particular position in the aligned sequences, the amino acid residue is identical between the sequences. “Similarity” indicates that, at any particular position in the aligned sequences, the amino acid residue is of a similar type between the sequences. Degrees of identity and similarity can be readily calculated (Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). Preferably, percentage identity, as referred to herein, is as determined using BLAST version 2.1.3 using the default parameters specified by the NCBI (the National Center for Biotechnology Information; http://www.ncbi.nlm.nih.gov/) [Blosum 62 matrix; gap open penalty=11 and gap extension penalty=1].

Homologous polypeptides therefore include natural biological variants (for example, allelic variants or geographical variations within the species from which the polypeptides are derived) and mutants (such as mutants containing amino acid substitutions, insertions or deletions) of the INSP171 polypeptides. Such mutants may include polypeptides in which one or more of the amino acid residues are substituted with a conserved or non-conserved amino acid residue (preferably a conserved amino acid residue) and such substituted amino acid residue may or may not be one encoded by the genetic code. Typical such substitutions are among Ala, Val, Leu and Ile; among Ser and Thr; among the acidic residues Asp and Glu; among Asn and Gln; among the basic residues Lys and Arg; or among the aromatic residues Phe and Tyr. Particularly preferred are variants in which several, i.e. between 5 and 10, 1 and 5, 1 and 3, 1 and 2 or just 1 amino acids are substituted, deleted or added in any combination. Especially preferred are silent substitutions, additions and deletions, which do not alter the properties and activities of the protein. Also especially preferred in this regard are conservative substitutions.

Such mutants also include polypeptides in which one or more of the amino acid residues includes a substituent group.

In accordance with the present invention, any substitution should be preferably a “conservative” or “safe” substitution, which is commonly defined a substitution introducing an amino acids having sufficiently similar chemical properties (e.g. a basic, positively charged amino acid should be replaced by another basic, positively charged amino acid), in order to preserve the structure and the biological function of the molecule.

The literature provide many models on which the selection of conservative amino acids substitutions can be performed on the basis of statistical and physico-chemical studies on the sequence and/or the structure of proteins (Rogov S I and Nekrasov A N, 2001). Protein design experiments have shown that the use of specific subsets of amino acids can produce foldable and active proteins, helping in the classification of amino acid “synonymous” substitutions which can be more easily accommodated in protein structure, and which can be used to detect functional and structural homologs and paralogs (Murphy L R et al., 2000). The groups of synonymous amino acids and the groups of more preferred synonymous amino acids are shown in Table 1.

Specific, non-conservative mutations can be also introduced in the polypeptides of the invention with different purposes. Mutations reducing the affinity of the Reeler domain containing protein may increase its ability to be reused and recycled, potentially increasing its therapeutic potency (Robinson C R, 2002). Immunogenic epitopes eventually present in the polypeptides of the invention can be exploited for developing vaccines (Stevanovic S, 2002), or eliminated by modifying their sequence following known methods for selecting mutations for increasing protein stability, and correcting them (van den Burg B and Eijsink V, 2002; WO 02/05146, WO 00/34317, WO 98/52976).

Preferred alternative, synonymous groups for amino acids derivatives included in peptide mimetics are those defined in Table 2. A non-exhaustive list of amino acid derivatives also include aminoisobutyric acid (Aib), hydroxyproline (Hyp), 1,2,3,4-tetrahydro-isoquinoline-3-COOH, indoline-2carboxylic acid, 4-difluoro-proline, L-thiazolidine-4-carboxylic acid, L-homoproline, 3,4-dehydro-proline, 3,4-dihydroxy-phenylalanine, cyclohexyl-glycine, and phenylglycine.

By “amino acid derivative” is intended an amino acid or amino acid-like chemical entity other than one of the 20 genetically encoded naturally occurring amino acids. In particular, the amino acid derivative may contain substituted or non-substituted, linear, branched, or cyclic alkyl moieties, and may include one or more heteroatoms. The amino acid derivatives can be made de novo or obtained from commercial sources (Calbiochem-Novabiochem A G, Switzerland; Bachem, USA).

Various methodologies for incorporating unnatural amino acids derivatives into proteins, using both in vitro and in vivo translation systems, to probe and/or improve protein structure and function are disclosed in the literature (Dougherty D A, 2000). Techniques for the synthesis and the development of peptide mimetics, as well as non-peptide mimetics, are also well known in the art (Golebiowski A et al., 2001; Hruby V J and Balse P M, 2000; Sawyer T K, in “Structure Based Drug Design”, edited by Veerapandian P, Marcel Dekker Inc., pg. 557-663, 1997).

Typically, greater than 80% identity between two polypeptides is considered to be an indication of functional equivalence. Preferably, functionally equivalent polypeptides of the first aspect of the invention have a degree of sequence identity with the INSP171 polypeptide, or with active fragments thereof, of greater than 80%. More preferred polypeptides have degrees of identity of greater than 90%, 95%, 98% or 99%, respectively.

The functionally-equivalent polypeptides of the first aspect of the invention may also be polypeptides which have been identified using one or more techniques of structural alignment. For example, the Inpharmatica Genome Threader technology that forms one aspect of the search tools used to generate the Biopendium search database may be used (see PCT application published as WO 01/69507) to identify polypeptides of presently-unknown function which, while having low sequence identity as compared to the INSP171 polypeptides, are predicted to be related molecules by virtue of sharing significant structural homology with the INSP171 polypeptide sequences. By “significant structural homology” is meant that the Inpharmatica Genome Threader predicts two proteins to share structural homology with a certainty of 10% and above.

The polypeptides of the first aspect of the invention also include fragments of the INSP171 polypeptides and fragments of the functional equivalents of these polypeptides, provided that those fragments retain INSP171 activity, or have an antigenic determinant in common with these polypeptides.

As used herein, the term “fragment” refers to a polypeptide having an amino acid sequence that is the same as part, but not all, of the amino acid sequence of the INSP171 polypeptides or one of their functional equivalents. The fragments should comprise at least n consecutive amino acids from the sequence and, depending on the particular sequence, n preferably is 7 or more (for example, 8, 10, 12, 14, 16, 18, 20 or more). Small fragments may form an antigenic determinant. Fragments according to the invention may be 1-100 amino acids in length, preferably, 5-50, more preferably 7-20 amino acids.

Nucleic acids according to the invention are preferably 100-1000 nucleotides in length, preferably 200-900 nucleotides, preferably 300-700, preferably 400-500, preferably 410-450 nucleotides in length. Polypeptides according to the invention are preferably 10-500 amino acids in length, preferably 50-400, preferably 100-300, preferably 200-250 amino acids in length.

Fragments of the full length INSP171 polypeptides may consist of combinations of neighbouring exon sequences in the INSP171 polypeptide sequences, respectively. Alternatively, fragments of the full length INSP171 polypeptides may consist of combinations of different domains of the INSP171 polypeptide, for example extracellular, transmembrane and intracellular domains. Such fragments are included in the present invention.

Such fragments may be “free-standing”, i.e. not part of or fused to other amino acids or polypeptides, or they may be comprised within a larger polypeptide of which they form a part or region. When comprised within a larger polypeptide, the fragment of the invention most preferably forms a single continuous region. For instance, certain preferred embodiments relate to a fragment having a pre- and/or pro-polypeptide region fused to the amino terminus of the fragment and/or an additional region fused to the carboxyl terminus of the fragment. However, several fragments may be comprised within a single larger polypeptide.

The polypeptides of the present invention or their immunogenic fragments (comprising at least one antigenic determinant) can be used to generate ligands, such as polyclonal or monoclonal antibodies, that are immunospecific for the polypeptides. Such antibodies may be employed to isolate or to identify clones expressing the polypeptides of the invention or to purify the polypeptides by affinity chromatography. The antibodies may also be employed as diagnostic or therapeutic aids, amongst other applications, as will be apparent to the skilled reader.

The term “immunospecific” means that the antibodies have substantially greater affinity for the polypeptides of the invention than their affinity for other related polypeptides in the prior art. As used herein, the term “antibody” refers to intact molecules as well as to fragments thereof, such as Fab, F(ab′)₂ and Fv, which are capable of binding to the antigenic determinant in question. Such antibodies thus bind to the polypeptides of the first aspect of the invention.

By “substantially greater affinity” we mean that there is a measurable increase in the affinity for a polypeptide of the invention as compared with the affinity for known cell-surface receptors.

Preferably, the affinity is at least 1.5-fold, 2-fold, 5-fold 10-fold, 100-fold, 10³-fold, 10⁴-fold, 10⁵-fold, 10⁶-fold or greater for a polypeptide of the invention than for known polypeptides containing Reeler domains.

Preferably, there is a measurable increase in the affinity for a polypeptide of the invention as compared with known Reeler domain containing proteins.

If polyclonal antibodies are desired, a selected mammal, such as a mouse, rabbit, goat or horse, may be immunised with a polypeptide of the first aspect of the invention. The polypeptide used to immunize the animal can be derived by recombinant DNA technology or can be synthesized chemically. If desired, the polypeptide can be conjugated to a carrier protein. Commonly used carriers to which the polypeptides may be chemically coupled include bovine serum albumin, thyroglobulin and keyhole limpet hemocyanin. The coupled polypeptide is then used to immunize the animal. Serum from the immunised animal is collected and treated according to known procedures, for example by immunoaffinity chromatography.

Monoclonal antibodies to the polypeptides of the first aspect of the invention can also be readily produced by one skilled in the art. The general methodology for making monoclonal antibodies using hybridoma technology is well known (see, for example, Kohler, G. and Milstein, C., Nature 256: 495-497 (1975); Kozbor et al., Immunology Today 4: 72 (1983); Cole et al., 77-96 in Monoclonal Antibodies and Cancer Therapy, Alan R. Liss, Inc. (1985).

Panels of monoclonal antibodies produced against the polypeptides of the first aspect of the invention can be screened for various properties, i.e., for isotype, epitope, affinity, etc. Monoclonal antibodies are particularly useful in purification of the individual polypeptides against which they are directed. Alternatively, genes encoding the monoclonal antibodies of interest may be isolated from hybridomas, for instance by PCR techniques known in the art, and cloned and expressed in appropriate vectors.

Chimeric antibodies, in which non-human variable regions are joined or fused to human constant regions (see, for example, Liu et al., Proc. Natl. Acad. Sci. USA, 84, 3439 (1987)), may also be of use.

The antibody may be modified to make it less immunogenic in an individual, for example by humanization (see Jones et al., Nature, 321, 522 (1986); Verhoeyen et al., Science, 239, 1534 (1988); Kabat et al., J. Immunol., 147, 1709 (1991); Queen et al., Proc. Natl Acad. Sci. USA, 86, 10029 (1989); Gorman et al., Proc. Natl. Acad. Sci. USA, 88, 34181 (1991); and Hodgson et al., Bio/Technology, 9, 421 (1991)). The term “humanized antibody”, as used herein, refers to antibody molecules in which the CDR amino acids and selected other amino acids in the variable domains of the heavy and/or light chains of a non-human donor antibody have been substituted in place of the equivalent amino acids in a human antibody. The humanized antibody thus closely resembles a human antibody but has the binding ability of the donor antibody.

In a further alternative, the antibody may be a “bispecific” antibody, that is an antibody having two different antigen-binding domains, each domain being directed against a different epitope.

Phage display technology may be utilised to select genes which encode antibodies with binding activities towards the polypeptides of the invention either from repertoires of PCR amplified V-genes of lymphocytes from humans screened for possessing the relevant antibodies, or from naive libraries (McCafferty, J. et al., (1990), Nature 348, 552-554; Marks, J. et al., (1992) Biotechnology 10, 779-783). The affinity of these antibodies can also be improved by chain shuffling (Clackson, T. et al., (1991) Nature 352, 624-628).

Antibodies generated by the above techniques, whether polyclonal or monoclonal, have additional utility in that they may be employed as reagents in immunoassays, radioimmunoassays (RIA) or enzyme-linked immunosorbent assays (ELISA). In these applications, the antibodies can be labelled with an analytically-detectable reagent such as a radioisotope, a fluorescent molecule or an enzyme.

Preferred nucleic acid molecules of the second and third aspects of the invention are those which encode a polypeptide sequences as recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38 or SEQ ID NO:40, and functionally equivalent polypeptides. These nucleic acid molecules may be used in the methods and applications described herein. The nucleic acid molecules of the invention preferably comprise at least n consecutive nucleotides from the sequences disclosed herein where, depending on the particular sequence, n is 10 or more (for example, 12, 14, 15, 18, 20, 25, 30, 35, 40 or more).

The nucleic acid molecules of the invention also include sequences that are complementary to nucleic acid molecules described above (for example, for antisense or probing purposes).

Nucleic acid molecules of the present invention may be in the form of RNA, such as mRNA, or in the form of DNA, including, for instance cDNA, synthetic DNA or genomic DNA. Such nucleic acid molecules may be obtained by cloning, by chemical synthetic techniques or by a combination thereof. The nucleic acid molecules can be prepared, for example, by chemical synthesis using techniques such as solid phase phosphoramidite chemical synthesis, from genomic or cDNA libraries or by separation from an organism. RNA molecules may generally be generated by the in vitro or in vivo transcription of DNA sequences.

The nucleic acid molecules may be double-stranded or single-stranded. Single-stranded DNA may be the coding strand, also known as the sense strand, or it may be the non-coding strand, also referred to as the anti-sense strand.

The term “nucleic acid molecule” also includes analogues of DNA and RNA, such as those containing modified backbones, and peptide nucleic acids (PNA). The term “PNA”, as used herein, refers to an antisense molecule or an anti-gene agent which comprises an oligonucleotide of at least five nucleotides in length linked to a peptide backbone of amino acid residues, which preferably ends in lysine. The terminal lysine confers solubility to the composition. PNAs may be pegylated to extend their lifespan in a cell, where they preferentially bind complementary single stranded DNA and RNA and stop transcript elongation (Nielsen, P. E. et al. (1993) Anticancer Drug Des. 8:53-63).

A nucleic acid molecule which encodes the polypeptide of SEQ ID NO:2 may be identical to the coding sequence of the nucleic acid molecule shown in SEQ ID NO:1. A nucleic acid molecule which encodes the polypeptide of SEQ ID NO:4 may be identical to the coding sequence of the nucleic acid molecule shown in SEQ ID NO:3. A nucleic acid molecule which encodes the polypeptide of SEQ ID NO:6 may be identical to the coding sequence of the nucleic acid molecule shown in SEQ ID NO:5. A nucleic acid molecule which encodes the polypeptide of SEQ ID NO:8 may be identical to the coding sequence of the nucleic acid molecule shown in SEQ ID NO:7. A nucleic acid molecule which encodes the polypeptide of SEQ ID NO:10 may be identical to the coding sequence of the nucleic acid molecule shown in SEQ ID NO:9. A nucleic acid molecule which encodes the polypeptide of SEQ ID NO:12 may be identical to the coding sequence of the nucleic acid molecule shown in SEQ ID NO:11. A nucleic acid molecule which encodes the polypeptide of SEQ ID NO:14 may be identical to the coding sequence of the nucleic acid molecule shown in SEQ ID NO:13. A nucleic acid molecule which encodes the polypeptide of SEQ ID NO:16 may be identical to the coding sequence of the nucleic acid molecule shown in SEQ ID NO:15. A nucleic acid molecule which encodes the polypeptide of SEQ ID NO:18 may be identical to the coding sequence of the nucleic acid molecule shown in SEQ ID NO:17. A nucleic acid molecule which encodes the polypeptide of SEQ ID NO:20 may be identical to the coding sequence of the nucleic acid molecule shown in SEQ ID NO:19. A nucleic acid molecule which encodes the polypeptide of SEQ ID NO:22 may be identical to the coding sequence of the nucleic acid molecule shown in SEQ ID NO:21. A nucleic acid molecule which encodes the polypeptide of SEQ ID NO:24 may be identical to the coding sequence of the nucleic acid molecule shown in SEQ ID NO:23. A nucleic acid molecule which encodes the polypeptide of SEQ ID NO:26 may be identical to the coding sequence of the nucleic acid molecule shown in SEQ ID NO:25. A nucleic acid molecule which encodes the polypeptide of SEQ ID NO:28 may be identical to the coding sequence of the nucleic acid molecule shown in SEQ ID NO:27. A nucleic acid molecule which encodes the polypeptide of SEQ ID NO:30 may be identical to the coding sequence of the nucleic acid molecule shown in SEQ ID NO:29. A nucleic acid molecule which encodes the polypeptide of SEQ ID NO:32 may be identical to the coding sequence of the nucleic acid molecule shown in SEQ ID NO:31. A nucleic acid molecule which encodes the polypeptide of SEQ ID NO:34 may be identical to the coding sequence of the nucleic acid molecule shown in SEQ ID NO:33. A nucleic acid molecule which encodes the polypeptide of SEQ ID NO:36 may be identical to the coding sequence of the nucleic acid molecule shown in SEQ ID NO:35. A nucleic acid molecule which encodes the polypeptide of SEQ ID NO:38 may be identical to the coding sequence of the nucleic acid molecule shown in SEQ ID NO:37. A nucleic acid molecule which encodes the polypeptide of SEQ ID NO:40 may be identical to the coding sequence of the nucleic acid molecule shown in SEQ ID NO:39.

These molecules also may have a different sequence which, as a result of the degeneracy of the genetic code, encodes a polypeptide of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38 or SEQ ID NO:40. Such nucleic acid molecules may include, but are not limited to, the coding sequence for the mature polypeptide by itself; the coding sequence for the mature polypeptide and additional coding sequences, such as those encoding a leader or secretory sequence, such as a pro-, pre- or prepro-polypeptide sequence; the coding sequence of the mature polypeptide, with or without the aforementioned additional coding sequences, together with further additional, non-coding sequences, including non-coding 5′ and 3′ sequences, such as the transcribed, non-translated sequences that play a role in transcription (including termination signals), ribosome binding and mRNA stability. The nucleic acid molecules may also include additional sequences which encode additional amino acids, such as those which provide additional functionalities.

The nucleic acid molecules of the second and third aspects of the invention may also encode the fragments or the functional equivalents of the polypeptides and fragments of the first aspect of the invention. Such a nucleic acid molecule may be a naturally-occurring variant such as a naturally-occurring allelic variant, or the molecule may be a variant that is not known to occur naturally. Such non-naturally occurring variants of the nucleic acid molecule may be made by mutagenesis techniques, including those applied to nucleic acid molecules, cells or organisms.

Among variants in this regard are variants that differ from the aforementioned nucleic acid molecules by nucleotide substitutions, deletions or insertions. The substitutions, deletions or insertions may involve one or more nucleotides. The variants may be altered in coding or non-coding regions or both. Alterations in the coding regions may produce conservative or non-conservative amino acid substitutions, deletions or insertions.

The nucleic acid molecules of the invention can also be engineered, using methods generally known in the art, for a variety of reasons, including modifying the cloning, processing, and/or expression of the gene product (the polypeptide). DNA shuffling by random fragmentation and PCR reassembly of gene fragments and synthetic oligonucleotides are included as techniques which may be used to engineer the nucleotide sequences. Site-directed mutagenesis may be used to insert new restriction sites, alter glycosylation patterns, change codon preference, produce splice variants, introduce mutations and so forth.

Nucleic acid molecules which encode a polypeptide of the first aspect of the invention may be ligated to a heterologous sequence so that the combined nucleic acid molecule encodes a fusion protein. Such combined nucleic acid molecules are included within the second or third aspects of the invention. For example, to screen peptide libraries for inhibitors of the activity of the polypeptide, it may be useful to express, using such a combined nucleic acid molecule, a fusion protein that can be recognised by a commercially-available antibody. A fusion protein may also be engineered to contain a cleavage site located between the sequence of the polypeptide of the invention and the sequence of a heterologous protein so that the polypeptide may be cleaved and purified away from the heterologous protein.

The nucleic acid molecules of the invention also include antisense molecules that are partially complementary to nucleic acid molecules encoding polypeptides of the present invention and that therefore hybridize to the encoding nucleic acid molecules (hybridization). Such antisense molecules, such as oligonucleotides, can be designed to recognise, specifically bind to and prevent transcription of a target nucleic acid encoding a polypeptide of the invention, as will be known by those of ordinary skill in the art (see, for example, Cohen, J. S., Trends in Pharm. Sci., 10, 435 (1989), Okano, J. Neurochem. 56, 560 (1991); O'Connor, J. Neurochem 56, 560 (1991); Lee et al., Nucleic Acids Res 6, 3073 (1979); Cooney et al., Science 241, 456 (1988); Dervan et al., Science 251, 1360 (1991)).

The term “hybridization” as used here refers to the association of two nucleic acid molecules with one another by hydrogen bonding. Typically, one molecule will be fixed to a solid support and the other will be free in solution. Then, the two molecules may be placed in contact with one another under conditions that favour hydrogen bonding. Factors that affect this bonding include: the type and volume of solvent; reaction temperature; time of hybridization; agitation; agents to block the non-specific attachment of the liquid phase molecule to the solid support (Denhardt's reagent or BLOTTO); the concentration of the molecules; use of compounds to increase the rate of association of molecules (dextran sulphate or polyethylene glycol); and the stringency of the washing conditions following hybridization (see Sambrook et al. [supra]).

The inhibition of hybridization of a completely complementary molecule to a target molecule may be examined using a hybridization assay, as known in the art (see, for example, Sambrook et al [supra]). A substantially homologous molecule will then compete for and inhibit the binding of a completely homologous molecule to the target molecule under various conditions of stringency, as taught in Wahl, G. M. and S. L. Berger (1987; Methods Enzymol. 152:399-407) and Kimmel, A. R. (1987; Methods Enzymol. 152:507-511).

“Stringency” refers to conditions in a hybridization reaction that favour the association of very similar molecules over association of molecules that differ. High stringency hybridisation conditions are defined as overnight incubation at 42° C. in a solution comprising 50% formamide, 5×SSC (150 mM NaCl, 15 mM trisodium citrate), 50 mM sodium phosphate (pH7.6), 5×Denhardts solution, 10% dextran sulphate, and 20 microgram/ml denatured, sheared salmon sperm DNA, followed by washing the filters in 0.1×SSC at approximately 65° C. Low stringency conditions involve the hybridisation reaction being carried out at 35° C. (see Sambrook et al. [supra]). Preferably, the conditions used for hybridization are those of high stringency.

Preferred embodiments of this aspect of the invention are nucleic acid molecules that are at least 70% identical over their entire length to nucleic acid molecules encoding the INSP171 polypeptides (SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38 or SEQ ID NO:40) and nucleic acid molecules that are substantially complementary to such nucleic acid molecules. Preferably, a nucleic acid molecule according to this aspect of the invention comprises a region that is at least 80% identical over its entire length to the nucleic acid molecules having the sequence produced by SEQ ID NO:1, SEQ ID NO:3, SEQ ID NO:5, SEQ ID NO:7, SEQ ID NO:9, SEQ ID NO:11, SEQ ID NO:13 SEQ ID NO:15, SEQ ID NO:17 SEQ ID NO:19, SEQ ID NO:21, SEQ ID NO:23, SEQ ID NO:25 SEQ ID NO:27, SEQ ID NO:29, SEQ ID NO:31, SEQ ID NO:33 SEQ ID NO:35, SEQ ID NO:37 or SEQ ID NO:39 or a nucleic acid molecule that is complementary thereto. In this regard, nucleic acid molecules at least 90%, preferably at least 95%, more preferably at least 98% or 99% identical over their entire length to the same are particularly preferred. Preferred embodiments in this respect are nucleic acid molecules that encode polypeptides which retain substantially the same biological function or activity as the INSP171 polypeptides.

The invention also provides a process for detecting a nucleic acid molecule of the invention, comprising the steps of: (a) contacting a nucleic probe according to the invention with a biological sample under hybridizing conditions to form duplexes; and (b) detecting any such duplexes that are formed.

As discussed additionally below in connection with assays that may be utilised according to the invention, a nucleic acid molecule as described above may be used as a hybridization probe for RNA, cDNA or genomic DNA, in order to isolate full-length cDNAs and genomic clones encoding the INSP171 polypeptides and to isolate cDNA and genomic clones of homologous or orthologous genes that have a high sequence similarity to the gene encoding this polypeptide.

In this regard, the following techniques, among others known in the art, may be utilised and are discussed below for purposes of illustration. Methods for DNA sequencing and analysis are well known and are generally available in the art and may, indeed, be used to practice many of the embodiments of the invention discussed herein. Such methods may employ such enzymes as the Klenow fragment of DNA polymerase I, Sequenase (US Biochemical Corp, Cleveland, Ohio), Taq polymerase (Perkin Elmer), thermostable T7 polymerase (Amersham, Chicago, Ill.), or combinations of polymerases and proof-reading exonucleases such as those found in the ELONGASE Amplification System marketed by Gibco/BRL (Gaithersburg, Md.). Preferably, the sequencing process may be automated using machines such as the Hamilton Micro Lab 2200 (Hamilton, Reno, Nev.), the Peltier Thermal Cycler (PTC200; MJ Research, Watertown, Mass.) and the ABI Catalyst and 373 and 377 DNA Sequencers (Perkin Elmer).

One method for isolating a nucleic acid molecule encoding a polypeptide with an equivalent function to that of the INSP171 polypeptides is to probe a genomic or cDNA library with a natural or artificially-designed probe using standard procedures that are recognised in the art (see, for example, “Current Protocols in Molecular Biology”, Ausubel et al. (eds). Greene Publishing Association and John Wiley Interscience, New York, 1989, 1992). Probes comprising at least 15, preferably at least 30, and more preferably at least 50, contiguous bases that correspond to, or are complementary to, nucleic acid sequences from the appropriate encoding gene (SEQ ID NO:1, SEQ ID NO:3, SEQ ID NO:5, SEQ ID NO:7, SEQ ID NO:9, SEQ ID NO:11, SEQ ID NO:13 SEQ ID NO:15, SEQ ID NO:17 SEQ ID NO:19 SEQ ID NO:21, SEQ ID NO:23, SEQ ID NO:25 SEQ ID NO:27, SEQ ID NO:29, SEQ ID NO:31, SEQ ID NO:33 SEQ ID NO:35, SEQ ID NO:37 or SEQ ID NO:39) are particularly useful probes. Such probes may be labelled with an analytically-detectable reagent to facilitate their identification. Useful reagents include, but are not limited to, radioisotopes, fluorescent dyes and enzymes that are capable of catalysing the formation of a detectable product. Using these probes, the ordinarily skilled artisan will be capable of isolating complementary copies of genomic DNA, cDNA or RNA polynucleotides encoding proteins of interest from human, mammalian or other animal sources and screening such sources for related sequences, for example, for additional members of the family, type and/or subtype.

In many cases, isolated cDNA sequences will be incomplete, in that the region encoding the polypeptide will be cut short, normally at the 5′ end. Several methods are available to obtain full length cDNAs, or to extend short cDNAs. Such sequences may be extended utilising a partial nucleotide sequence and employing various methods known in the art to detect upstream sequences such as promoters and regulatory elements. For example, one method which may be employed is based on the method of Rapid Amplification of cDNA Ends (RACE; see, for example, Frohman et al., PNAS USA 85, 8998-9002, 1988). Recent modifications of this technique, exemplified by the Marathon™ technology (Clontech Laboratories Inc.), for example, have significantly simplified the search for longer cDNAs. A slightly different technique, termed “restriction-site” PCR, uses universal primers to retrieve unknown nucleic acid sequence adjacent a known locus (Sarkar, G. (1993) PCR Methods Applic. 2:318-322). Inverse PCR may also be used to amplify or to extend sequences using divergent primers based on a known region (Triglia, T. et al. (1988) Nucleic Acids Res. 16:8186). Another method which may be used is capture PCR which involves PCR amplification of DNA fragments adjacent a known sequence in human and yeast artificial chromosome DNA (Lagerstrom, M. et al. (1991) PCR Methods Applic., 1, 111-119). Another method which may be used to retrieve unknown sequences is that of Parker, J. D. et al. (1991); Nucleic Acids Res. 19:3055-3060). Additionally, one may use PCR, nested primers, and PromoterFinder™ libraries to walk genomic DNA (Clontech, Palo Alto, Calif.). This process avoids the need to screen libraries and is useful in finding intron/exon junctions.

When screening for full-length cDNAs, it is preferable to use libraries that have been size-selected to include larger cDNAs. Also, random-primed libraries are preferable, in that they will contain more sequences that contain the 5′ regions of genes. Use of a randomly primed library may be especially preferable for situations in which an oligo d(T) library does not yield a full-length cDNA. Genomic libraries may be useful for extension of sequence into 5′ non-transcribed regulatory regions.

In one embodiment of the invention, the nucleic acid molecules of the present invention may be used for chromosome localisation. In this technique, a nucleic acid molecule is specifically targeted to, and can hybridize with, a particular location on an individual human chromosome. The mapping of relevant sequences to chromosomes according to the present invention is an important step in the confirmatory correlation of those sequences with the gene-associated disease. Once a sequence has been mapped to a precise chromosomal location, the physical position of the sequence on the chromosome can be correlated with genetic map data. Such data are found in, for example, V. McKusick, Mendelian Inheritance in Man (available on-line through Johns Hopkins University Welch Medical Library). The relationships between genes and diseases that have been mapped to the same chromosomal region are then identified through linkage analysis (coinheritance of physically adjacent genes). This provides valuable information to investigators searching for disease genes using positional cloning or other gene discovery techniques. Once the disease or syndrome has been crudely localised by genetic linkage to a particular genomic region, any sequences mapping to that area may represent associated or regulatory genes for further investigation. The nucleic acid molecule may also be used to detect differences in the chromosomal location due to translocation, inversion, etc. among normal, carrier, or affected individuals.

The nucleic acid molecules of the present invention are also valuable for tissue localisation. Such techniques allow the determination of expression patterns of the polypeptide in tissues by detection of the mRNAs that encode them. These techniques include in situ hybridization techniques and nucleotide amplification techniques, such as PCR. Results from these studies provide an indication of the normal functions of the polypeptide in the organism. In addition, comparative studies of the normal expression pattern of mRNAs with that of mRNAs encoded by a mutant gene provide valuable insights into the role of mutant polypeptides in disease. Such inappropriate expression may be of a temporal, spatial or quantitative nature.

Gene silencing approaches may also be undertaken to down-regulate endogenous expression of a gene encoding a polypeptide of the invention. RNA interference (RNAi) (Elbashir, S M et al., Nature 2001, 411, 494-498) is one method of sequence specific post-transcriptional gene silencing that may be employed. Short dsRNA oligonucleotides are synthesised in vitro and introduced into a cell. The sequence specific binding of these dsRNA oligonucleotides triggers the degradation of target mRNA, reducing or ablating target protein expression.

Efficacy of the gene silencing approaches assessed above may be assessed through the measurement of polypeptide expression (for example, by Western blotting), and at the RNA level using TaqMan-based methodologies.

The vectors of the present invention comprise nucleic acid molecules of the invention and may be cloning or expression vectors. The host cells of the invention, which may be transformed, transfected or transduced with the vectors of the invention may be prokaryotic or eukaryotic.

The polypeptides of the invention may be prepared in recombinant form by expression of their encoding nucleic acid molecules in vectors contained within a host cell. Such expression methods are well known to those of skill in the art and many are described in detail by Sambrook et al (supra) and Fernandez & Hoeffler (1998, eds. “Gene expression systems. Using nature for the art of expression”. Academic Press, an Diego, London, Boston, New York, Sydney, Tokyo, Toronto).

Generally, any system or vector that is suitable to maintain, propagate or express nucleic acid molecules to produce a polypeptide in the required host may be used. The appropriate nucleotide sequence may be inserted into an expression system by any of a variety of well-known and routine techniques, such as, for example, those described in Sambrook et al., (supra). Generally, the encoding gene can be placed under the control of a control element such as a promoter, ribosome binding site (for bacterial expression) and, optionally, an operator, so that the DNA sequence encoding the desired polypeptide is transcribed into RNA in the transformed host cell.

Examples of suitable expression systems include, for example, chromosomal, episomal and virus-derived systems, including, for example, vectors derived from: bacterial plasmids, bacteriophage, transposons, yeast episomes, insertion elements, yeast chromosomal elements, viruses such as baculoviruses, papova viruses such as SV40, vaccinia viruses, adenoviruses, fowl pox viruses, pseudorabies viruses and retroviruses, or combinations thereof, such as those derived from plasmid and bacteriophage genetic elements, including cosmids and phagemids. Human artificial chromosomes (HACs) may also be employed to deliver larger fragments of DNA than can be contained and expressed in a plasmid. The vectors pEAK12d, pDEST12.2, pEAK12d_INSP171-6HIS, pDEST12.2_INSP171-6HIS, pEAK12d_INSP171SV1-6HIS, pDEST12.2_INSP171SV1-6HIS, pEAK12d_INSP171SV2-6HIS, pDEST12.2_INSP171SV2-6HIS, pDEST12.2INSP171SV3-6HIS, pEAK12d_INSP171SV4-6HIS and pDEST12.2INSP171SV4-6HIS are preferred examples of suitable vectors for use in accordance with the aspects of this invention relating to INSP171.

Particularly suitable expression systems include microorganisms such as bacteria transformed with recombinant bacteriophage, plasmid or cosmid DNA expression vectors; yeast transformed with yeast expression vectors; insect cell systems infected with virus expression vectors (for example, baculovirus); plant cell systems transformed with virus expression vectors (for example, cauliflower mosaic virus, CaMV; tobacco mosaic virus, TMV) or with bacterial expression vectors (for example, Ti or pBR322 plasmids); or animal cell systems. Cell-free translation systems can also be employed to produce the polypeptides of the invention.

Introduction of nucleic acid molecules encoding a polypeptide of the present invention into host cells can be effected by methods described in many standard laboratory manuals, such as Davis et al., Basic Methods in Molecular Biology (1986) and Sambrook et al., [supra]. Particularly suitable methods include calcium phosphate transfection, DEAE-dextran mediated transfection, transvection, microinjection, cationic lipid-mediated transfection, electroporation, transduction, scrape loading, ballistic introduction or infection (see Sambrook et al., 1989 [supra]; Ausubel et al., 1991 [supra]; Spector, Goldman & Leinwald, 1998). In eukaryotic cells, expression systems may either be transient (for example, episomal) or permanent (chromosomal integration) according to the needs of the system.

The encoding nucleic acid molecule may or may not include a sequence encoding a control sequence, such as a signal peptide or leader sequence, as desired, for example, for secretion of the translated polypeptide into the lumen of the endoplasmic reticulum, into the periplasmic space or into the extracellular environment. These signals may be endogenous to the polypeptide or they may be heterologous signals. Leader sequences can be removed by the bacterial host in post-translational processing.

In addition to control sequences, it may be desirable to add regulatory sequences that allow for regulation of the expression of the polypeptide relative to the growth of the host cell. Examples of regulatory sequences are those which cause the expression of a gene to be increased or decreased in response to a chemical or physical stimulus, including the presence of a regulatory compound or to various temperature or metabolic conditions. Regulatory sequences are those non-translated regions of the vector, such as enhancers, promoters and 5′ and 3′ untranslated regions. These interact with host cellular proteins to carry out transcription and translation. Such regulatory sequences may vary in their strength and specificity. Depending on the vector system and host utilised, any number of suitable transcription and translation elements, including constitutive and inducible promoters, may be used. For example, when cloning in bacterial systems, inducible promoters such as the hybrid lacZ promoter of the Bluescript phagemid (Stratagene, LaJolla, Calif.) or pSportI™ plasmid (Gibco BRL) and the like may be used. The baculovirus polyhedrin promoter may be used in insect cells. Promoters or enhancers derived from the genomes of plant cells (for example, heat shock, RUBISCO and storage protein genes) or from plant viruses (for example, viral promoters or leader sequences) may be cloned into the vector. In mammalian cell systems, promoters from mammalian genes or from mammalian viruses are preferable. If it is necessary to generate a cell line that contains multiple copies of the sequence, vectors based on SV40 or EBV may be used with an appropriate selectable marker.

An expression vector is constructed so that the particular nucleic acid coding sequence is located in the vector with the appropriate regulatory sequences, the positioning and orientation of the coding sequence with respect to the regulatory sequences being such that the coding sequence is transcribed under the “control” of the regulatory sequences, i.e., RNA polymerase which binds to the DNA molecule at the control sequences transcribes the coding sequence. In some cases it may be necessary to modify the sequence so that it may be attached to the control sequences with the appropriate orientation; i.e., to maintain the reading frame.

The control sequences and other regulatory sequences may be ligated to the nucleic acid coding sequence prior to insertion into a vector. Alternatively, the coding sequence can be cloned directly into an expression vector that already contains the control sequences and an appropriate restriction site.

For long-term, high-yield production of a recombinant polypeptide, stable expression is preferred. For example, cell lines which stably express the polypeptide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells that successfully express the introduced sequences. Resistant clones of stably transformed cells may be proliferated using tissue culture techniques appropriate to the cell type.

Mammalian cell lines available as hosts for expression are known in the art and include many immortalised cell lines available from the American Type Culture Collection (ATCC) including, but not limited to, Chinese hamster ovary (CHO), HeLa, baby hamster kidney (BHK), monkey kidney (COS), C127, 3T3, BHK, HEK 293, Bowes melanoma and human hepatocellular carcinoma (for example Hep G2) cells and a number of other cell lines.

In the baculovirus system, the materials for baculovirus/insect cell expression systems are commercially available in kit form from, inter alia, Invitrogen, San Diego Calif. (the “MaxBac” kit). These techniques are generally known to those skilled in the art and are described fully in Summers and Smith, Texas Agricultural Experiment Station Bulletin No. 1555 (1987). Particularly suitable host cells for use in this system include insect cells such as Drosophila S2 and Spodoptera Sf9 cells.

There are many plant cell culture and whole plant genetic expression systems known in the art. Examples of suitable plant cellular genetic expression systems include those described in U.S. Pat. No. 5,693,506; U.S. Pat. No. 5,659,122; and U.S. Pat. No. 5,608,143. Additional examples of genetic expression in plant cell culture have been described by Zenk, Phytochemistry 30, 3861-3863 (1991).

In particular, all plants from which protoplasts can be isolated and cultured to give whole regenerated plants can be utilised, so that whole plants are recovered which contain the transferred gene. Practically all plants can be regenerated from cultured cells or tissues, including but not limited to all major species of sugar cane, sugar beet, cotton, fruit and other trees, legumes and vegetables.

Examples of particularly preferred bacterial host cells include streptococci, staphylococci, E. coli, Streptomyces and Bacillus subtilis cells.

Examples of particularly suitable host cells for fungal expression include yeast cells (for example, S. cerevisiae) and Aspergillus cells.

Any number of selection systems are known in the art that may be used to recover transformed cell lines. Examples include the herpes simplex virus thymidine kinase (Wigler, M. et al. (1977) Cell 11:223-32) and adenine phosphoribosyltransferase (Lowy, I. et al. (1980) Cell 22:817-23) genes that can be employed in tk⁻ or aprt^{±} cells, respectively.

Also, antimetabolite, antibiotic or herbicide resistance can be used as the basis for selection; for example, dihydrofolate reductase (DHFR) that confers resistance to methotrexate (Wigler, M. et al. (1980) Proc. Natl. Acad. Sci. 77:3567-70); npt, which confers resistance to the aminoglycosides neomycin and G-418 (Colbere-Garapin, F. et al (1981) J. Mol. Biol. 150:1-14) and als or pat, which confer resistance to chlorsulfuron and phosphinotricin acetyltransferase, respectively. Additional selectable genes have been described, examples of which will be clear to those of skill in the art.

Although the presence or absence of marker gene expression suggests that the gene of interest is also present, its presence and expression may need to be confirmed. For example, if the relevant sequence is inserted within a marker gene sequence, transformed cells containing the appropriate sequences can be identified by the absence of marker gene function. Alternatively, a marker gene can be placed in tandem with a sequence encoding a polypeptide of the invention under the control of a single promoter. Expression of the marker gene in response to induction or selection usually indicates expression of the tandem gene as well.

Alternatively, host cells that contain a nucleic acid sequence encoding a polypeptide of the invention and which express said polypeptide may be identified by a variety of procedures known to those of skill in the art. These procedures include, but are not limited to, DNA-DNA or DNA-RNA hybridizations and protein bioassays, for example, fluorescence activated cell sorting (FACS) or immunoassay techniques (such as the enzyme-linked immunosorbent assay [ELISA] and radioimmunoassay [RIA]), that include membrane, solution, or chip based technologies for the detection and/or quantification of nucleic acid or protein (see Hampton, R. et al. (1990) Serological Methods, a Laboratory Manual, APS Press, St Paul, Minn.) and Maddox, D. E. et al. (1983) J. Exp. Med, 158, 1211-1216).

A wide variety of labels and conjugation techniques are known by those skilled in the art and may be used in various nucleic acid and amino acid assays. Means for producing labelled hybridization or PCR probes for detecting sequences related to nucleic acid molecules encoding polypeptides of the present invention include oligolabelling, nick translation, end-labelling or PCR amplification using a labelled polynucleotide. Alternatively, the sequences encoding the polypeptide of the invention may be cloned into a vector for the production of an mRNA probe. Such vectors are known in the art, are commercially available, and may be used to synthesise RNA probes in vitro by addition of an appropriate RNA polymerase such as T7, T3 or SP6 and labelled nucleotides. These procedures may be conducted using a variety of commercially available kits (Pharmacia & Upjohn, (Kalamazoo, Mich.); Promega (Madison Wis.); and U.S. Biochemical Corp., Cleveland, Ohio)).

Suitable reporter molecules or labels, which may be used for ease of detection, include radionuclides, enzymes and fluorescent, chemiluminescent or chromogenic agents as well as substrates, cofactors, inhibitors, magnetic particles, and the like.

Nucleic acid molecules according to the present invention may also be used to create transgenic animals, particularly rodent animals. Such transgenic animals form a further aspect of the present invention. This may be done locally by modification of somatic cells, or by germ line therapy to incorporate heritable modifications. Such transgenic animals may be particularly useful in the generation of animal models for drug molecules effective as modulators of the polypeptides of the present invention.

The polypeptide can be recovered and purified from recombinant cell cultures by well-known methods including ammonium sulphate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography is particularly useful for purification. Well known techniques for refolding proteins may be employed to regenerate an active conformation when the polypeptide is denatured during isolation and or purification.

Specialised vector constructions may also be used to facilitate purification of proteins, as desired, by joining sequences encoding the polypeptides of the invention to a nucleotide sequence encoding a polypeptide domain that will facilitate purification of soluble proteins. Examples of such purification-facilitating domains include metal chelating peptides such as histidine-tryptophan modules that allow purification on immobilised metals, protein A domains that allow purification on immobilised immunoglobulin, and the domain utilised in the FLAGS extension/affinity purification system (Immunex Corp., Seattle, Wash.). The inclusion of cleavable linker sequences such as those specific for Factor XA or enterokinase (Invitrogen, San Diego, Calif.) between the purification domain and the polypeptide of the invention may be used to facilitate purification. One such expression vector provides for expression of a fusion protein containing the polypeptide of the invention fused to several histidine residues preceding a thioredoxin or an enterokinase cleavage site. The histidine residues facilitate purification by IMAC (immobilised metal ion affinity chromatography as described in Porath, J. et al. (1992), Prot. Exp. Puff. 3: 263-281) while the thioredoxin or enterokinase cleavage site provides a means for purifying the polypeptide from the fusion protein. A discussion of vectors which contain fusion proteins is provided in Kroll, D. J. et al. (1993; DNA Cell Biol. 12:441-453).

If the polypeptide is to be expressed for use in screening assays, generally it is preferred that it be secreted into the culture medium of the host cell in which it is expressed. In this event, the polypeptides of the invention may be purified from the culture medium may be harvested prior to use in the screening assay, for example using standard protein purification techniques such as gel exclusion chromatography, ion-exchange chromatography or affinity chromatography. Examples of suitable methods of protein purification are provided in the Examples herein. If polypeptide is produced intracellularly, the cells must first be lysed before the polypeptide is recovered.

As indicated above, the present invention also provides novel targets and methods for the screening of drug candidates or leads. These screening methods include binding assays and/or functional assays, and may be performed in vitro, in cell systems or in animals.

In this regard, a particular object of this invention resides in the use of an INSP171 polypeptide as a target for screening candidate drugs for treating or preventing Reeler domain containing protein related disorders.

Another object of this invention resides in methods of selecting biologically active compounds, said methods comprising contacting a candidate compound with a INSP171 gene or polypeptide, and selecting compounds that bind said gene or polypeptide.

A further other object of this invention resides in methods of selecting biologically active compounds, said method comprising contacting a candidate compound with recombinant host cell expressing a INSP171 polypeptide with a candidate compound, and selecting compounds that bind said INSP171 polypeptide at the surface of said cells and/or that modulate the activity of the INSP171 polypeptide.

A “biologically active” compound denotes any compound having biological activity in a subject, preferably therapeutic activity, more preferably a compound having Reeler domain activity, and further preferably a compound that can be used for treating INSP171 related disorders, or as a lead to develop drugs for treating a Reeler domain containing protein related disorder. A “biologically active” compound preferably is a compound that modulates the activity of INSP171.

The above methods may be conducted in vitro, using various devices and conditions, including with immobilized reagents, and may further comprise an additional step of assaying the activity of the selected compounds in a model of Reeler domain containing protein related disorder, such as an animal model.

Preferred selected compounds are agonists of INSP171, i.e., compounds that can bind to INSP171 and mimic the activity of an endogenous ligand thereof.

A further object of this invention resides in a method of selecting biologically active compounds, said method comprising contacting in vitro a test compound with a INSP171 polypeptide according to the present invention and determining the ability of said test compound to modulate the activity of said INSP171 polypeptide.

A further object of this invention resides in a method of selecting biologically active compounds, said method comprising contacting in vitro a test compound with a INSP171 gene according to the present invention and determining the ability of said test compound to modulate the expression of said INSP171 gene, preferably to stimulate expression thereof.

In another embodiment, this invention relates to a method of screening, selecting or identifying active compounds, particularly compounds active on multiple sclerosis or related disorders, the method comprising contacting a test compound with a recombinant host cell comprising a reporter construct, said reporter construct comprising a reporter gene under the control of a INSP171 gene promoter, and selecting the test compounds that modulate (e.g. stimulate or reduce, preferably stimulate) expression of the reporter gene.

The polypeptide of the invention can be used to screen libraries of compounds in any of a variety of drug screening techniques. Such compounds may activate (agonize) or inhibit (antagonize) the level of expression of the gene or the activity of the polypeptide of the invention and form a further aspect of the present invention. Preferred compounds are effective to alter the expression of a natural gene which encodes a polypeptide of the first aspect of the invention or to regulate the activity of a polypeptide of the first aspect of the invention.

Agonist or antagonist compounds may be isolated from, for example, cells, cell-free preparations, chemical libraries or natural product mixtures. These agonists or antagonists may be natural or modified substrates, ligands, enzymes, receptors or structural or functional mimetics. For a suitable review of such screening techniques, see Coligan et al., Current Protocols in Immunology 1(2):Chapter 5 (1991).

Binding to a target gene or polypeptide provides an indication as to the ability of the compound to modulate the activity of said target, and thus to affect a pathway leading to Reeler domain containing protein related disorder in a subject. The determination of binding may be performed by various techniques, such as by labelling of the candidate compound, by competition with a labelled reference ligand, etc. For in vitro binding assays, the polypeptides may be used in essentially pure form, in suspension, immobilized on a support, or expressed in a membrane (intact cell, membrane preparation, liposome, etc.).

Modulation of activity includes, without limitation, stimulation of the surface expression of the INSP171 receptor, modulation of multimerization of said receptor (e.g., the formation of multimeric complexes with other sub-units), etc. The cells used in the assays may be any recombinant cell (i.e., any cell comprising a recombinant nucleic acid encoding a INSP171 polypeptide) or any cell that expresses an endogenous INSP171 polypeptide. Examples of such cells include, without limitation, prokaryotic cells (such as bacteria) and eukaryotic cells (such as yeast cells, mammalian cells, insect cells, plant cells, etc.). Specific examples include E. coli, Pichia pastoris, Hansenula polymorpha, Schizosaccharomyces pombe, Kluyveromyces or Saccharomyces yeasts, mammalian cell lines (e.g., Vero cells, CHO cells, 3T3 cells, COS cells, etc.) as well as primary or established mammalian cell cultures (e.g., produced from fibroblasts, embryonic cells, epithelial cells, nervous cells, adipocytes, etc.).

Compounds that are most likely to be good antagonists are molecules that bind to the polypeptide of the invention without inducing the biological effects of the polypeptide upon binding to it. Potential antagonists include small organic molecules, peptides, polypeptides and antibodies that bind to the polypeptide of the invention and thereby inhibit or extinguish its activity. In this fashion, binding of the polypeptide to normal cellular binding molecules may be inhibited, such that the normal biological activity of the polypeptide is prevented.

The polypeptide of the invention that is employed in such a screening technique may be free in solution, affixed to a solid support, borne on a cell surface or located intracellularly. In general, such screening procedures may involve using appropriate cells or cell membranes that express the polypeptide that are contacted with a test compound to observe binding, or stimulation or inhibition of a functional response. The functional response of the cells contacted with the test compound is then compared with control cells that were not contacted with the test compound. Such an assay may assess whether the test compound results in a signal generated by activation of the polypeptide, using an appropriate detection system. Inhibitors of activation are generally assayed in the presence of a known agonist and the effect on activation by the agonist in the presence of the test compound is observed.

A preferred method for identifying an agonist or antagonist compound of a polypeptide of the present invention comprises:

(a) contacting a cell expressing (optionally on the surface thereof) the polypeptide according to the first aspect of the invention, the polypeptide being associated with a second component capable of providing a detectable signal in response to the binding of a compound to the polypeptide, with a compound to be screened under conditions to permit binding to the polypeptide; and
(b) determining whether the compound binds to and activates or inhibits the polypeptide by measuring the level of a signal generated from the interaction of the compound with the polypeptide.

Methods for generating detectable signals in the types of assays described herein will be known to those of skill in the art. A particular example is cotransfecting a construct expressing a polypeptide according to the invention, or a fragment such as the LBD, in fusion with the GAL4 DNA binding domain, into a cell together with a reporter plasmid, an example of which is pFR-Luc (Stratagene Europe, Amsterdam, The Netherlands). This particular plasmid contains a synthetic promoter with five tandem repeats of GAL4 binding sites that control the expression of the luciferase gene. When a potential ligand is added to the cells, it will bind the GAL4-polypeptide fusion and induce transcription of the luciferase gene. The level of the luciferase expression can be monitored by its activity using a luminescence reader (see, for example, Lehman et al. JBC 270, 12953, 1995; Pawar et al. JBC, 277, 39243, 2002).

A further preferred method for identifying an agonist or antagonist of a polypeptide of the invention comprises:

(a) contacting a labelled or unlabeled compound with the polypeptide immobilized on any solid support (for example beads, plates, matrix support, chip) and detection of the compound by measuring the label or the presence of the compound itself; or
(b) contacting a cell expressing on the surface thereof the polypeptide, by means of artificially anchoring it to the cell membrane, or by constructing a chimeric receptor being associated with a second component capable of providing a detectable signal in response to the binding of a compound to the polypeptide, with a compound to be screened under conditions to permit binding to the polypeptide; and
(c) determining whether the compound binds to and activates or inhibits the polypeptide by comparing the level of a signal generated from the interaction of the compound with the polypeptide with the level of a signal in the absence of the compound.

For example, a method such as FRET detection of ligand bound to the polypeptide in the presence of peptide co-activators (Norris et al, Science 285, 744, 1999) might be used.

A further preferred method for identifying an agonist or antagonist of a polypeptide of the invention comprises:

(a) contacting a cell expressing (optionally on the surface thereof) the polypeptide, the polypeptide being associated with a second component capable of providing a detectable signal in response to the binding of a compound to the polypeptide, with a compound to be screened under conditions to permit binding to the polypeptide; and
(b) determining whether the compound binds to and activates or inhibits the polypeptide by comparing the level of a signal generated from the interaction of the compound with the polypeptide with the level of a signal in the absence of the compound.

In further preferred embodiments, the general methods that are described above may further comprise conducting the identification of agonist or antagonist in the presence of labelled or unlabelled ligand for the polypeptide.

In another embodiment of the method for identifying agonist or antagonist of a polypeptide of the present invention comprises:

determining the inhibition of binding of a ligand to cells which express a polypeptide of the invention (and which optionally have a polypeptide of the invention on the surface thereof), or to cell membranes containing such a polypeptide, in the presence of a candidate compound under conditions to permit binding to the polypeptide, and determining the amount of ligand bound to the polypeptide. A compound capable of causing reduction of binding of a ligand is considered to be an agonist or antagonist. Preferably the ligand is labelled.

More particularly, a method of screening for a polypeptide antagonist or agonist compound comprises the steps of:

(a) incubating a labelled ligand with a whole cell expressing a polypeptide according to the invention, optionally on the cell surface, or a cell membrane containing a polypeptide of the invention,
(b) measuring the amount of labelled ligand bound to the whole cell or the cell membrane;
(c) adding a candidate compound to a mixture of labelled ligand and the whole cell or the cell membrane of step (a) and allowing the mixture to attain equilibrium;
(d) measuring the amount of labelled ligand bound to the whole cell or the cell membrane after step (c); and
(e) comparing the difference in the labelled ligand bound in step (b) and (d), such that the compound which causes the reduction in binding in step (d) is considered to be an agonist or antagonist.

Similarly, there is provided a method of screening for a polypeptide antagonist or agonist compound which comprises the steps of:

(a) incubating a labelled ligand with a polypeptide according to the invention on any solid support or the cell surface, or a cell membrane containing a polypeptide of the invention.
(b) measuring the amount of labelled ligand bound to the polypeptide on the solid support, whole cell or the cell membrane;
(c) adding a candidate compound to a mixture of labelled ligand and immobilized polypeptide on the solid support, the whole cell or the cell membrane of step (a) and allowing the mixture to attain equilibrium;
(d) measuring the amount of labelled ligand bound to the immobilized polypeptide or the whole cell or the cell membrane after step (c); and
(e) comparing the difference in the labelled ligand bound in step (b) and (d), such that the compound which causes the reduction in binding in step (d) is considered to be an agonist or antagonist.

The polypeptides may be found to modulate a variety of physiological and pathological processes in a dose-dependent manner in the above-described assays. Thus, the “functional equivalents” of the polypeptides of the invention include polypeptides that exhibit any of the same modulatory activities in the above-described assays in a dose-dependent manner. Although the degree of dose-dependent activity need not be identical to that of the polypeptides of the invention, preferably the “functional equivalents” will exhibit substantially similar dose-dependence in a given activity assay compared to the polypeptides of the invention.

In certain of the embodiments described above, simple binding assays may be used, in which the adherence of a test compound to a surface bearing the polypeptide is detected by means of a label directly or indirectly associated with the test compound or in an assay involving competition with a labelled competitor. In another embodiment, competitive drug screening assays may be used, in which neutralizing antibodies that are capable of binding the polypeptide specifically compete with a test compound for binding. In this manner, the antibodies can be used to detect the presence of any test compound that possesses specific binding affinity for the polypeptide.

Persons skilled in the art will be able to devise assays for identifying modulators of a polypeptide of the invention. Of interest in this regard is Lokker N A et al., J. Biol. Chem., 1997, Dec. 26; 272(52):33037-44, which reports an example of an assay to identify antagonists (in this case neutralizing antibodies).

Assays may also be designed to detect the effect of added test compounds on the production of mRNA encoding the polypeptide in cells. For example, an ELISA may be constructed that measures secreted or cell-associated levels of polypeptide using monoclonal or polyclonal antibodies by standard methods known in the art, and this can be used to search for compounds that may inhibit or enhance the production of the polypeptide from suitably manipulated cells or tissues. The formation of binding complexes between the polypeptide and the compound being tested may then be measured.

Assay methods that are also included within the terms of the present invention are those that involve the use of the genes and polypeptides of the invention in overexpression or ablation assays. Such assays involve the manipulation of levels of these genes/polypeptides in cells and assessment of the impact of this manipulation event on the physiology of the manipulated cells. For example, such experiments reveal details of signalling and metabolic pathways in which the particular genes/polypeptides are implicated, generate information regarding the identities of polypeptides with which the studied polypeptides interact and provide clues as to methods by which related genes and proteins are regulated.

Another technique for drug screening which may be used provides for high throughput screening of compounds having suitable binding affinity to the polypeptide of interest (see International patent application WO84/03564). In this method, large numbers of different small test compounds are synthesised on a solid substrate, which may then be reacted with the polypeptide of the invention and washed. One way of immobilising the polypeptide is to use non-neutralizing antibodies. Bound polypeptide may then be detected using methods that are well known in the art. Purified polypeptide can also be coated directly onto plates for use in the aforementioned drug screening techniques.

The polypeptide of the invention may be used to identify membrane-bound or soluble receptors, through standard receptor binding techniques that are known in the art, such as ligand binding and crosslinking assays in which the polypeptide is labelled with a radioactive isotope, is chemically modified, or is fused to a peptide sequence that facilitates its detection or purification, and incubated with a source of the putative receptor (for example, a composition of cells, cell membranes, cell supernatants, tissue extracts, or bodily fluids). The efficacy of binding may be measured using biophysical techniques such as surface plasmon resonance (supplied by Biacore AB, Uppsala, Sweden) and spectroscopy. Binding assays may be used for the purification and cloning of the receptor, but may also identify agonists and antagonists of the polypeptide, that compete with the binding of the polypeptide to its receptor. Standard methods for conducting screening assays are well understood in the art.

In another embodiment, this invention relates to the use of a INSP171 polypeptide or fragment thereof, whereby the fragment is preferably a INSP171 gene-specific fragment, for isolating or generating an agonist or stimulator of the INSP171 polypeptide for the treatment of an immune related disorder, wherein said agonist or stimulator is selected from the group consisting of:

1. a specific antibody or fragment thereof including: a) a chimeric, b) a humanized or c) a fully human antibody, as well as;
2. a bispecific or multispecific antibody,
3. a single chain (e.g. scFv) or
4. single domain antibody, or
5. a peptide- or non-peptide mimetic derived from said antibodies or
6. an antibody-mimetic such as a) an anticalin or b) a fibronectin-based binding molecule (e.g. trinectin or adnectin).

The generation of peptide- or non-peptide mimetics from antibodies is known in the art (Saragovi et al., 1991 and Saragovi et al., 1992).

Anticalins are also known in the art (Vogt et al., 2004). Fibronectin-based binding molecules are described in U.S. Pat. No. 6,818,418 and WO2004/029224.

Furthermore, the test compound may be of various origin, nature and composition, such as any small molecule, nucleic acid, lipid, peptide, polypeptide including an antibody such as a chimeric, humanized or fully human antibody or an antibody fragment, peptide- or non-peptide mimetic derived therefrom as well as a bispecific or multispecific antibody, a single chain (e.g. scFv) or single domain antibody or an antibody-mimetic such as an anticalin or fibronectin-based binding molecule (e.g. trinectin or adnectin), etc., in isolated form or in mixture or combinations.

The invention also includes a screening kit useful in the methods for identifying agonists, antagonists, ligands, receptors, substrates, enzymes, that are described above.

The invention includes the agonists, antagonists, ligands, receptors, substrates and enzymes, and other compounds which modulate the activity or antigenicity of the polypeptide of the invention discovered by the methods that are described above.

As mentioned above, it is envisaged that the various moieties of the invention (i.e. the polypeptides of the first aspect of the invention, a nucleic acid molecule of the second or third aspect of the invention, a vector of the fourth aspect of the invention, a host cell of the fifth aspect of the invention, a ligand of the sixth aspect of the invention, a compound of the seventh aspect of the invention) may be useful in the therapy or diagnosis of diseases. To assess the utility of the moieties of the invention for treating or diagnosing a disease one or more of the following assays may be carried out. Note that although some of the following assays refer to the test compound as being a protein/polypeptide, a person skilled in the art will readily be able to adapt the following assays so that the other moieties of the invention may also be used as the “test compound”.

The invention also provides pharmaceutical compositions comprising a polypeptide, nucleic acid, ligand or compound of the invention in combination with a suitable pharmaceutical carrier. These compositions may be suitable as therapeutic or diagnostic reagents, as vaccines, or as other immunogenic compositions, as outlined in detail below.

According to the terminology used herein, a composition containing a polypeptide, nucleic acid, ligand or compound [X] is “substantially free of” impurities [herein, Y] when at least 85% by weight of the total X+Y in the composition is X. Preferably, X comprises at least about 90% by weight of the total of X+Y in the composition, more preferably at least about 95%, 98% or even 99% by weight.

The pharmaceutical compositions should preferably comprise a therapeutically effective amount of the polypeptide, nucleic acid molecule, ligand, or compound of the invention. The term “therapeutically effective amount” as used herein refers to an amount of a therapeutic agent needed to treat, ameliorate, or prevent a targeted disease or condition, or to exhibit a detectable therapeutic or preventative effect. For any compound, the therapeutically effective dose can be estimated initially either in cell culture assays, for example, of neoplastic cells, or in animal models, usually mice, rabbits, dogs, or pigs. The animal model may also be used to determine the appropriate concentration range and route of administration. Such information can then be used to determine useful doses and routes for administration in humans.

The precise effective amount for a human subject will depend upon the severity of the disease state, general health of the subject, age, weight, and gender of the subject, diet, time and frequency of administration, drug combination(s), reaction sensitivities, and tolerance/response to therapy. This amount can be determined by routine experimentation and is within the judgement of the clinician. Generally, an effective dose will be from 0.01 mg/kg to 50 mg/kg, preferably 0.05 mg/kg to 10 mg/kg. Compositions may be administered individually to a patient or may be administered in combination with other agents, drugs or hormones.

A pharmaceutical composition may also contain a pharmaceutically acceptable carrier, for administration of a therapeutic agent. Such carriers include antibodies and other polypeptides, genes and other therapeutic agents such as liposomes, provided that the carrier does not itself induce the production of antibodies harmful to the individual receiving the composition, and which may be administered without undue toxicity. Suitable carriers may be large, slowly metabolised macromolecules such as proteins, polysaccharides, polylactic acids, polyglycolic acids, polymeric amino acids, amino acid copolymers and inactive virus particles.

Pharmaceutically acceptable salts can be used therein, for example, mineral acid salts such as hydrochlorides, hydrobromides, phosphates, sulphates, and the like; and the salts of organic acids such as acetates, propionates, malonates, benzoates, and the like. A thorough discussion of pharmaceutically acceptable carriers is available in Remington's Pharmaceutical Sciences (Mack Pub. Co., N.J. 1991).

Pharmaceutically acceptable carriers in therapeutic compositions may additionally contain liquids such as water, saline, glycerol and ethanol. Additionally, auxiliary substances, such as wetting or emulsifying agents, pH buffering substances, and the like, may be present in such compositions. Such carriers enable the pharmaceutical compositions to be formulated as tablets, pills, dragees, capsules, liquids, gels, syrups, slurries, suspensions, and the like, for ingestion by the patient.

Once formulated, the compositions of the invention can be administered directly to the subject. The subjects to be treated can be animals; in particular, human subjects can be treated.

The pharmaceutical compositions utilised in this invention may be administered by any number of routes including, but not limited to, oral, intravenous, intramuscular, intra-arterial, intramedullary, intrathecal, intraventricular, transdermal or transcutaneous applications (for example, see WO98/20734), subcutaneous, intraperitoneal, intranasal, enteral, topical, sublingual, intravaginal or rectal means. Gene guns or hyposprays may also be used to administer the pharmaceutical compositions of the invention. Typically, the therapeutic compositions may be prepared as injectables, either as liquid solutions or suspensions; solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule.

If the activity of the polypeptide of the invention is in excess in a particular disease state, several approaches are available. One approach comprises administering to a subject an inhibitor compound (antagonist) as described above, along with a pharmaceutically acceptable carrier in an amount effective to inhibit the function of the polypeptide, such as by blocking the binding of ligands, substrates, enzymes, receptors, or by inhibiting a second signal, and thereby alleviating the abnormal condition. Preferably, such antagonists are antibodies. Most preferably, such antibodies are chimeric and/or humanized to minimise their immunogenicity, as described previously.

In another approach, soluble forms of the polypeptide that retain binding affinity for the ligand, substrate, enzyme, receptor, in question, may be administered. Typically, the polypeptide may be administered in the form of fragments that retain the relevant portions.

In an alternative approach, expression of the gene encoding the polypeptide can be inhibited using expression blocking techniques, such as the use of antisense nucleic acid molecules (as described above), either internally generated or separately administered. Modifications of gene expression can be obtained by designing complementary sequences or antisense molecules (DNA, RNA, or PNA) to the control, 5′ or regulatory regions (signal sequence, promoters, enhancers and introns) of the gene encoding the polypeptide. Similarly, inhibition can be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) In: Huber, B. E. and B. I. Carr, Molecular and Immunologic Approaches, Futura Publishing Co., Mt. Kisco, N.Y.). The complementary sequence or antisense molecule may also be designed to block translation of mRNA by preventing the transcript from binding to ribosomes. Such oligonucleotides may be administered or may be generated in situ from expression in vivo.

In addition, expression of the polypeptide of the invention may be prevented by using ribozymes specific to its encoding mRNA sequence. Ribozymes are catalytically active RNAs that can be natural or synthetic (see for example Usman, N, et al., Curr. Opin. Struct. Biol (1996) 6(4), 527-33). Synthetic ribozymes can be designed to specifically cleave mRNAs at selected positions thereby preventing translation of the mRNAs into functional polypeptide. Ribozymes may be synthesised with a natural ribose phosphate backbone and natural bases, as normally found in RNA molecules. Alternatively the ribozymes may be synthesised with non-natural backbones, for example, 2′-O-methyl RNA, to provide protection from ribonuclease degradation and may contain modified bases.

RNA molecules may be modified to increase intracellular stability and half-life. Possible modifications include, but are not limited to, the addition of flanking sequences at the 5′ and/or 3′ ends of the molecule or the use of phosphorothioate or 2′O-methyl rather than phosphodiesterase linkages within the backbone of the molecule. This concept is inherent in the production of PNAs and can be extended in all of these molecules by the inclusion of non-traditional bases such as inosine, queosine and butosine, as well as acetyl-, methyl-, thio- and similarly modified forms of adenine, cytidine, guanine, thymine and uridine which are not as easily recognised by endogenous endonucleases.

For treating abnormal conditions related to an under-expression of the polypeptide of the invention and its activity, several approaches are also available. One approach comprises administering to a subject a therapeutically effective amount of a compound that activates the polypeptide, i.e., an agonist as described above, to alleviate the abnormal condition. Alternatively, a therapeutic amount of the polypeptide in combination with a suitable pharmaceutical carrier may be administered to restore the relevant physiological balance of polypeptide.

Gene therapy may be employed to effect the endogenous production of the polypeptide by the relevant cells in the subject. Gene therapy is used to treat permanently the inappropriate production of the polypeptide by replacing a defective gene with a corrected therapeutic gene.

Gene therapy of the present invention can occur in vivo or ex vivo. Ex vivo gene therapy requires the isolation and purification of patient cells, the introduction of a therapeutic gene and introduction of the genetically altered cells back into the patient. In contrast, in vivo gene therapy does not require isolation and purification of a patient's cells.

The therapeutic gene is typically “packaged” for administration to a patient. Gene delivery vehicles may be non-viral, such as liposomes, or replication-deficient viruses, such as adenovirus as described by Berkner, K. L., in Curr. Top. Microbiol. Immunol., 158, 39-66 (1992) or adeno-associated virus (AAV) vectors as described by Muzyczka, N., in Curr. Top. Microbiol. Immunol., 158, 97-129 (1992) and U.S. Pat. No. 5,252,479. For example, a nucleic acid molecule encoding a polypeptide of the invention may be engineered for expression in a replication-defective retroviral vector. This expression construct may then be isolated and introduced into a packaging cell transduced with a retroviral plasmid vector containing RNA encoding the polypeptide, such that the packaging cell now produces infectious viral particles containing the gene of interest. These producer cells may be administered to a subject for engineering cells in vivo and expression of the polypeptide in vivo (see Chapter 20, Gene Therapy and other Molecular Genetic-based Therapeutic Approaches, (and references cited therein) in Human Molecular Genetics (1996), T Strachan and A P Read, BIOS Scientific Publishers Ltd).

Another approach is the administration of “naked DNA” in which the therapeutic gene is directly injected into the bloodstream or muscle tissue.

In situations in which the polypeptides or nucleic acid molecules of the invention are disease-causing agents, the invention provides that they can be used in vaccines to raise antibodies against the disease causing agent.

Vaccines according to the invention may either be prophylactic (i.e. to prevent infection) or therapeutic (i.e. to treat disease after infection). Such vaccines comprise immunizing antigen(s), immunogen(s), polypeptide(s), protein(s) or nucleic acid, usually in combination with pharmaceutically-acceptable carriers as described above, which include any carrier that does not itself induce the production of antibodies harmful to the individual receiving the composition. Additionally, these carriers may function as immunostimulating agents (“adjuvants”). Furthermore, the antigen or immunogen may be conjugated to a bacterial toxoid, such as a toxoid from diphtheria, tetanus, cholera, H. pylori, and other pathogens.

Since polypeptides may be broken down in the stomach, vaccines comprising polypeptides are preferably administered parenterally (for instance, subcutaneous, intramuscular, intravenous, or intradermal injection). Formulations suitable for parenteral administration include aqueous and non-aqueous sterile injection solutions which may contain anti-oxidants, buffers, bacteriostats and solutes which render the formulation isotonic with the blood of the recipient, and aqueous and non-aqueous sterile suspensions which may include suspending agents or thickening agents.

The vaccine formulations of the invention may be presented in unit-dose or multi-dose containers. For example, sealed ampoules and vials and may be stored in a freeze-dried condition requiring only the addition of the sterile liquid carrier immediately prior to use. The dosage will depend on the specific activity of the vaccine and can be readily determined by routine experimentation.

Genetic delivery of antibodies that bind to polypeptides according to the invention may also be effected, for example, as described in International patent application WO98/55607.

The technology referred to as jet injection (see, for example, www.powderject.com) may also be useful in the formulation of vaccine compositions.

A number of suitable methods for vaccination and vaccine delivery systems are described in International patent application WO00/29428.

This invention also relates to the use of nucleic acid molecules according to the present invention as diagnostic reagents. Detection of a mutated form of the gene characterised by the nucleic acid molecules of the invention which is associated with a dysfunction will provide a diagnostic tool that can add to, or define, a diagnosis of a disease, or susceptibility to a disease, which results from under-expression, over-expression or altered spatial or temporal expression of the gene. Individuals carrying mutations in the gene may be detected at the DNA level by a variety of techniques.

Nucleic acid molecules for diagnosis may be obtained from a subject's cells, such as from blood, urine, saliva, tissue biopsy or autopsy material. The genomic DNA may be used directly for detection or may be amplified enzymatically by using PCR, ligase chain reaction (LCR), strand displacement amplification (SDA), or other amplification techniques (see Saiki et al., Nature, 324, 163-166 (1986); Bej, et al., Crit. Rev. Biochem. Molec. Biol., 26, 301-334 (1991); Birkenmeyer et al., J. Virol. Meth., 35, 117-126 (1991); Van Brunt, J., Bio/Technology, 8, 291-294 (1990)) prior to analysis.

In one embodiment, this aspect of the invention provides a method of diagnosing a disease in a patient, comprising assessing the level of expression of a natural gene encoding a polypeptide according to the invention and comparing said level of expression to a control level, wherein a level that is different to said control level is indicative of disease. The method may comprise the steps of:
a) contacting a sample of tissue from the patient with a nucleic acid probe under stringent conditions that allow the formation of a hybrid complex between a nucleic acid molecule of the invention and the probe;
b) contacting a control sample with said probe under the same conditions used in step a);
c) and detecting the presence of hybrid complexes in said samples;
   wherein detection of levels of the hybrid complex in the patient sample that differ from levels of the hybrid complex in the control sample is indicative of disease.

A further aspect of the invention comprises a diagnostic method comprising the steps of:
a) obtaining a tissue sample from a patient being tested for disease;
b) isolating a nucleic acid molecule according to the invention from said tissue sample; and
c) diagnosing the patient for disease by detecting the presence of a mutation in the nucleic acid molecule which is associated with disease.

To aid the detection of nucleic acid molecules in the above-described methods, an amplification step, for example using PCR, may be included.

Deletions and insertions can be detected by a change in the size of the amplified product in comparison to the normal genotype. Point mutations can be identified by hybridizing amplified DNA to labelled RNA of the invention or alternatively, labelled antisense DNA sequences of the invention. Perfectly-matched sequences can be distinguished from mismatched duplexes by RNase digestion or by assessing differences in melting temperatures. The presence or absence of the mutation in the patient may be detected by contacting DNA with a nucleic acid probe that hybridises to the DNA under stringent conditions to form a hybrid double-stranded molecule, the hybrid double-stranded molecule having an unhybridized portion of the nucleic acid probe strand at any portion corresponding to a mutation associated with disease; and detecting the presence or absence of an unhybridized portion of the probe strand as an indication of the presence or absence of a disease-associated mutation in the corresponding portion of the DNA strand.

Such diagnostics are particularly useful for prenatal and even neonatal testing.

Point mutations and other sequence differences between the reference gene and “mutant” genes can be identified by other well-known techniques, such as direct DNA sequencing or single-strand conformational polymorphism, (see Orita et al., Genomics, 5, 874-879 (1989)). For example, a sequencing primer may be used with double-stranded PCR product or a single-stranded template molecule generated by a modified PCR. The sequence determination is performed by conventional procedures with radiolabelled nucleotides or by automatic sequencing procedures with fluorescent-tags. Cloned DNA segments may also be used as probes to detect specific DNA segments. The sensitivity of this method is greatly enhanced when combined with PCR. Further, point mutations and other sequence variations, such as polymorphisms, can be detected as described above, for example, through the use of allele-specific oligonucleotides for PCR amplification of sequences that differ by single nucleotides.

DNA sequence differences may also be detected by alterations in the electrophoretic mobility of DNA fragments in gels, with or without denaturing agents, or by direct DNA sequencing (for example, Myers et al., Science (1985) 230:1242). Sequence changes at specific locations may also be revealed by nuclease protection assays, such as RNase and S1 protection or the chemical cleavage method (see Cotton et al., Proc. Natl. Acad. Sci. USA (1985) 85: 4397-4401).

In addition to conventional gel electrophoresis and DNA sequencing, mutations such as microdeletions, aneuploidies, translocations, inversions, can also be detected by in situ analysis (see, for example, Keller et al., DNA Probes, 2nd Ed., Stockton Press, New York, N.Y., USA (1993)), that is, DNA or RNA sequences in cells can be analyzed for mutations without need for their isolation and/or immobilisation onto a membrane. Fluorescence in situ hybridization (FISH) is presently the most commonly applied method and numerous reviews of FISH have appeared (see, for example, Trachuck et al., Science, 250, 559-562 (1990), and Trask et al., Trends, Genet., 7, 171-154 (1991)).

In another embodiment of the invention, an array of oligonucleotide probes comprising a nucleic acid molecule according to the invention can be constructed to conduct efficient screening of genetic variants, mutations and polymorphisms. Array technology methods are well known and have general applicability and can be used to address a variety of questions in molecular genetics including gene expression, genetic linkage, and genetic variability (see for example: M. Chee et al., Science (1996), Vol 274, pp 610-613).

In one embodiment, the array is prepared and used according to the methods described in PCT application WO95/11995 (Chee et al); Lockhart, D. J. et al. (1996) Nat. Biotech. 14: 1675-1680); and Schena, M. et al. (1996) Proc. Natl. Acad. Sci. 93: 10614-10619). Oligonucleotide pairs may range from two to over one million. The oligomers are synthesized at designated areas on a substrate using a light-directed chemical process. The substrate may be paper, nylon or other type of membrane, filter, chip, glass slide or any other suitable solid support. In another aspect, an oligonucleotide may be synthesized on the surface of the substrate by using a chemical coupling procedure and an ink jet application apparatus, as described in PCT application WO95/25116 (Baldeschweiler et al). In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536 or 6144 oligonucleotides, or any other number between two and over one million which lends itself to the efficient use of commercially-available instrumentation.

In addition to the methods discussed above, diseases may be diagnosed by methods comprising determining, from a sample derived from a subject, an abnormally decreased or increased level of polypeptide or mRNA. Decreased or increased expression can be measured at the RNA level using any of the methods well known in the art for the quantitation of polynucleotides, such as, for example, nucleic acid amplification, for instance PCR, RT-PCR, RNase protection, Northern blotting and other hybridization methods.

Assay techniques that can be used to determine levels of a polypeptide of the present invention in a sample derived from a host are well-known to those of skill in the art and are discussed in some detail above (including radioimmunoassays, competitive-binding assays, Western Blot analysis and ELISA assays). This aspect of the invention provides a diagnostic method which comprises the steps of: (a) contacting a ligand as described above with a biological sample under conditions suitable for the formation of a ligand-polypeptide complex; and (b) detecting said complex.

Protocols such as ELISA, RIA, and FACS for measuring polypeptide levels may additionally provide a basis for diagnosing altered or abnormal levels of polypeptide expression. Normal or standard values for polypeptide expression are established by combining body fluids or cell extracts taken from normal mammalian subjects, preferably humans, with antibody to the polypeptide under conditions suitable for complex formation The amount of standard complex formation may be quantified by various methods, such as by photometric means.

Antibodies which specifically bind to a polypeptide of the invention may be used for the diagnosis of conditions or diseases characterised by expression of the polypeptide, or in assays to monitor patients being treated with the polypeptides, nucleic acid molecules, ligands and other compounds of the invention. Antibodies useful for diagnostic purposes may be prepared in the same manner as those described above for therapeutics. Diagnostic assays for the polypeptide include methods that utilise the antibody and a label to detect the polypeptide in human body fluids or extracts of cells or tissues. The antibodies may be used with or without modification, and may be labelled by joining them, either covalently or non-covalently, with a reporter molecule. A wide variety of reporter molecules known in the art may be used, several of which are described above.

Quantities of polypeptide expressed in subject, control and disease samples from biopsied tissues are compared with the standard values. Deviation between standard and subject values establishes the parameters for diagnosing disease. Diagnostic assays may be used to distinguish between absence, presence, and excess expression of polypeptide and to monitor regulation of polypeptide levels during therapeutic intervention. Such assays may also be used to evaluate the efficacy of a particular therapeutic treatment regimen in animal studies, in clinical trials or in monitoring the treatment of an individual patient.

A diagnostic kit of the present invention may comprise:

(a) a nucleic acid molecule of the present invention;
(b) a polypeptide of the present invention; or
(c) a ligand of the present invention.

In one aspect of the invention, a diagnostic kit may comprise a first container containing a nucleic acid probe that hybridises under stringent conditions with a nucleic acid molecule according to the invention; a second container containing primers useful for amplifying the nucleic acid molecule; and instructions for using the probe and primers for facilitating the diagnosis of disease. The kit may further comprise a third container holding an agent for digesting unhybridized RNA.

In an alternative aspect of the invention, a diagnostic kit may comprise an array of nucleic acid molecules, at least one of which may be a nucleic acid molecule according to the invention.

To detect polypeptide according to the invention, a diagnostic kit may comprise one or more antibodies that bind to a polypeptide according to the invention; and a reagent useful for the detection of a binding reaction between the antibody and the polypeptide.

Such kits will be of use in diagnosing a disease or susceptibility to disease, particularly diseases in which polypeptides containing Reeler domains are implicated. Such diseases include, but are not limited to neoplasm, cancer, brain tumour, glioma, bone tumor, lung tumor, breast tumour, prostate tumour, colon tumour, hemangioma, myeloproliferative disorder, leukemia, hematological disease, neutropenia, thrombocytopenia, angiogenesis disorders, dermatological disease, aging, wounds, burns, fibrosis, cardiovascular disease, restenosis, heart disease, peripheral vascular disease, coronary artery disease, oedema, thromboembolism, dysmenorrhea, endometriosis, pre-eclampsia, lung disease, COPD, asthma bone disease, renal disease, glomerulonephritis, liver disease, Crohn's disease, gastritis, ulcerative colitis, ulcer, immune disorder, autoimmune disease, arthritis, rheumatoid arthritis, psoriasis, epidermolysis bullosa, systemic lupus erythematosus, ankylosing spondylitis, Lyme disease, multiple sclerosis, neurodegeneration, stroke, brain/spinal cord injury, Alzheimer's disease, Parkinson's disease, motor neurone disease, neuromuscular disease, CNS inflammation, cerebellar degeneration, amylotrophic lateral sclerosis, head injury damage, and other neurological abnormalities, HIV, AIDS, cytomegalovirus infection and fungal infection.

Such kits will be of use in diagnosing a disease or susceptibility to disease, particularly diseases in which polypeptides containing Reeler domains are implicated. Particular examples of such diseases include, but are not limited to, neurological disorders such as, for example, schizophrenia, neurodegeneration, neurodevelopmental disease, stroke, brain/spinal cord injury, Alzheimer's disease, Parkinson's disease, motor neurone disease, neuromuscular disease, CNS inflammation, cerebellar degeneration, amylotrophic lateral sclerosis, head injury damage, and other neurological abnormalities, including Autosomal recessive lissencephaly with cerebellar hypoplasia and autistic disorders.

Various aspects and embodiments of the present invention will now be described in more detail by way of example, with particular reference to INSP171 polypeptides.

It will be appreciated that modification of detail may be made without departing from the scope of the invention.

### BRIEF DESCRIPTION OF THE FIGURES

FIG. 1: INSP171 cDNA and protein sequence. The position and sense of the PCR primers are indicated by arrows.

FIG. 2: Amino acid sequence alignment of INSP171 with the 4 splice variants. Signal peptide is shaded.

FIG. 3: The main differences between INSP171 mature polypeptide and INSP171-SV1, INSP171-SV2, INSP171-SV3 and INSP171-SV4 mature polypeptides. The Reeler domain is underlined and Asparagines predicted to be N-glycosylated using the NetNglyc server prediction tool are highlighted.

FIG. 4: Amino acid sequence alignment of the reeler domain INSP171, INSP171-SV1, human F-spondin (SwissProt Acc. No. Q9HCB6) and SDR2 (SwissProt Acc. No. P97301). Conserved residues are indicated by an asterisk.

FIG. 5: Nucleotide sequence with translation of the INSP171 PCR product cloned using primers INSP171-CP3 and INSP171-CP4. The position

FIG. 6: Nucleotide sequence with translation of the INSP171SV1 PCR product cloned using primers INSP171-CP3 and INSP171-CP4. The position and sense of the PCR primers are indicated by arrows.

FIG. 7: Nucleotide sequence with translation of the INSP171SV2 PCR product cloned using primers INSP171-CP3 and INSP171-CP4. The position and sense of the PCR primers are indicated by arrows.

FIG. 8: Nucleotide sequence with translation of the INSP171SV3 PCR product cloned using primers INSP171-CP3 and INSP171-CP4. The position and sense of the PCR primers are indicated by arrows.

FIG. 9: Nucleotide sequence with translation of the INSP171SV4 PCR product cloned using primers INSP171-CP3 and INSP171-CP4. The position and sense of the PCR primers are indicated by arrows.

TABLE 1

| | | More Preferred |
|---|---|---|
| Amino Acid | Synonymous Groups | Synonymous Groups |
| Ser | Gly, Ala, Ser, Thr, Pro | Thr, Ser |
| Arg | Asn, Lys, Gln, Arg, His | Arg, Lys, His |
| Leu | Phe, Ile, Val, Leu, Met | Ile, Val, Leu, Met |
| Pro | Gly, Ala, Ser, Thr, Pro | Pro |
| Thr | Gly, Ala, Ser, Thr, Pro | Thr, Ser |
| Ala | Gly, Thr, Pro, Ala, Ser | Gly, Ala |
| Val | Met, Phe, Ile, Leu, Val | Met, Ile, Val, Leu |
| Gly | Ala, Thr, Pro, Ser, Gly | Gly, Ala |
| Ile | Phe, Ile, Val, Leu, Met | Ile, Val, Leu, Met |
| Phe | Trp, Phe, Tyr | Tyr, Phe |
| Tyr | Trp, Phe, Tyr | Phe, Tyr |
| Cys | Ser, Thr, Cys | Cys |
| His | Asn, Lys, Gln, Arg, His | Arg, Lys, His |
| Gln | Glu, Asn, Asp, Gln | Asn, Gln |
| Asn | Glu, Asn, Asp, Gln | Asn, Gln |
| Lys | Asn, Lys, Gln, Arg, His | Arg, Lys, His |
| Asp | Glu, Asn, Asp, Gln | Asp, Glu |
| Glu | Glu, Asn, Asp, Gln | Asp, Glu |
| Met | Phe, Ile, Val, Leu, Met | Ile, Val, Leu, Met |
| Trp | Trp, Phe, Tyr | Trp |

TABLE 2

| Amino Acid | Synonymous Groups |
|---|---|
| Ser | D-Ser, Thr, D-Thr, allo-Thr, Met, D-Met, Met(O), D-Met(O), L- |
| | Cys, D-Cys |
| Arg | D-Arg, Lys, D-Lys, homo-Arg, D-homo-Arg, Met, Ile, D-.Met, D- |
| | Ile, Orn, D-Orn |
| Leu | D-Leu, Val, D-Val, AdaA, AdaG, Leu, D-Leu, Met, D-Met |
| Pro | D-Pro, L-I-thioazolidine-4-carboxylic acid, D-or L-1-oxazolidine- |
| | 4-carboxylic acid |
| Thr | D-Thr, Ser, D-Ser, allo-Thr, Met, D-Met, Met(O), D-Met(O), Val, |
| | D-Val |
| Ala | D-Ala, Gly, Aib, B-Ala, Acp, L-Cys, D-Cys |
| Val | D-Val, Leu, D-Leu, Ile, D-Ile, Met, D-Met, AdaA, AdaG |
| Gly | Ala, D-Ala, Pro, D-Pro, Aib, .beta.-Ala, Acp |
| Ile | D-Ile, Val, D-Val, AdaA, AdaG, Leu, D-Leu, Met, D-Met |
| Phe | D-Phe, Tyr, D-Thr, L-Dopa, His, D-His, Trp, D-Trp, Trans-3, 4, or |
| | 5-phenylproline, AdaA, AdaG, cis-3, 4, or 5-phenylproline, Bpa, |
| | D-Bpa |
| Tyr | D-Tyr, Phe, D-Phe, L-Dopa, His, D-His |
| Cys | D-Cys, S--Me--Cys, Met, D-Met, Thr, D-Thr |
| Gln | D-Gln, Asn, D-Asn, Glu, D-Glu, Asp, D-Asp |
| Asn | D-Asn, Asp, D-Asp, Glu, D-Glu, Gln, D-Gln |
| Lys | D-Lys, Arg, D-Arg, homo-Arg, D-homo-Arg, Met, D-Met, Ile, D- |
| | Ile, Orn, D-Orn |
| Asp | D-Asp, D-Asn, Asn, Glu, D-Glu, Gln, D-Gln |
| Glu | D-Glu, D-Asp, Asp, Asn, D-Asn, Gln, D-Gln |
| Met | D-Met, S--Me--Cys, Ile, D-Ile, Leu, D-Leu, Val, D-Val |

### EXAMPLES

### Example 1

### Identification and in Silico Analysis of INSP171

### 1.1 Identification of the Reeler Domain

The INSP171 polypeptide sequence, shown in SEQ ID NO:22, was used as a BLAST query against the NCBI non-redundant sequence database. The INSP171 polypeptide sequence with the alternative start point of Methonine 3 of SEQ ID NO:22 was also used as a BLAST query against the NCBI non-redundant sequence database. The top hits are all proteins that contain Reeler domains. The top hits all align to the query sequence with highly significant E-values, thus indicating a very high degree of confidence in the prediction. The results of this BLAST query indicate that the INSP171 polypeptide contains a Reeler domain and thus this protein can be predicted to possess biological activity that is characteristic of such a protein.

These results indicate that both predictions for the INSP171 polypeptide contain a Reeler domain and thus these proteins can be predicted to possess the biological activity characteristic of such a protein.

As described above, the INSP171 polypeptide is predicted to comprise a leader sequence that is cleaved between positions 23 and 24 of the sequence beginning at methionine 1 of SEQ ID NO: 22 (this falls between positions 20 and 21 of the sequence beginning at methionine 3 of SEQ ID NO: 22). The presence of a leader sequence is consistent with the INSP171 protein being a secreted protein.

On the basis of these experiments and the INSP171 sequence information provided herein, it is now possible to design experiments to detect the presence of the INSP171 transcript across a range of human tissue types to determine its tissue expression. In addition, it will be possible to design experiments to detect the presence of the INSP171 transcript across a range of normal and diseased tissues in order to establish more particularly the relevance of the INSP171 protein in a pathological context.

At the same time, the cloning of the INSP171 gene from human genomic DNA will allow the high level expression of the INSP171 protein in prokaryotic or eukaryotic expression systems and its subsequent purification and characterisation. For example, recombinant INSP171 may be used to generate INSP171-specific monoclonal or polyclonal antibodies which might then be used in the biochemical characterisation of INSP171. Alternatively, recombinant INSP171 may be used in a wide variety of screening assays, including those described above, and those described in Example 2 below.

### 1.2 Analysis of INSP171 Splice Variants

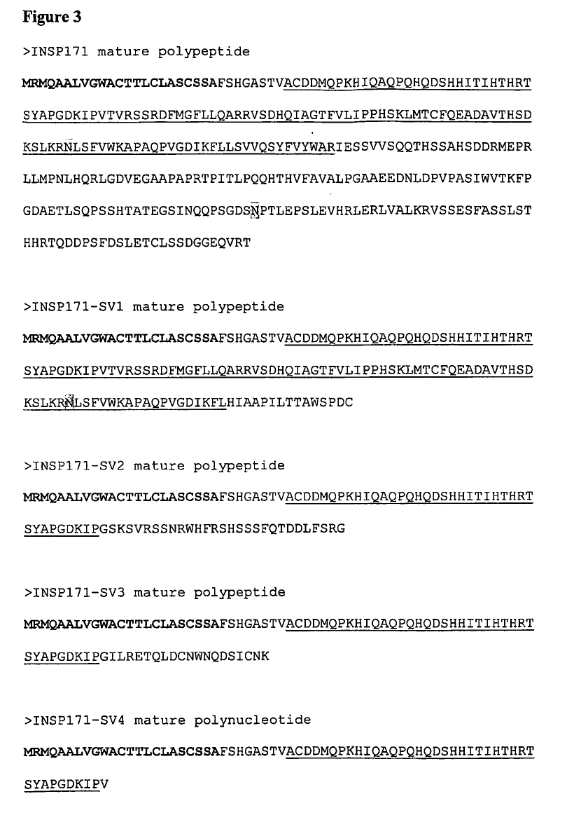

The main differences between INSP171 mature polypeptide and INSP171-SV1, INSP171-SV2, INSP171-SV3 and INSP171-SV4 mature polypeptides are shown in FIG. 3. The differences reside in the presence of a complete or partial Reeler domain (which is underlined) as well as the presence or absence of one or more Asparagines (which are highlighted) predicted to be N-glycosylated using the NetNglyc server prediction tool.

A complete Reeler domain has been predicted in INSP171 as well as two N-glycosylated Asparagine residues. INSP171-SV1 contains most of the Reeler domain however the last predicted N-Glycosylated Asparagine is absent. Whilst in INSP171-SV2, INSP171-SV3 and INSP171-SV4 only incomplete Reeler domains are present and they lack any predicted N-glycosylated Asparagines.

For domain annotation for FIG. 3 the INSP171 mature polypeptide and INSP171-SV1, INSP171-SV2, INSP171-SV3 and INSP171-SV4 mature polypeptide sequences were submitted to Interpro. A significant match to Reeler domain was predicted by Pfam for INSP171 and INSP171-SV1 mature polypeptides only; whilst no significant hits were identified for INSP171-SV2, INSP171-SV3 and INSP171-SV4. However, the respective incomplete Reeler domains were identified by manual inspection of the sequence and are highlighted for illustration purposes.

In terms of EST information, INSP171 is represented by one human EST from hepatocellular carcinoma (AV698927) and INSP171 and splice variants may be represented by two ESTs from endometrial carcinoma (BX114465.1, AI499628.1).

### Example 2

### Neurobiology Assays Suitable for Exploration of the Biological Relevance of INSP171 Function

A number of neurobiology-related assays have been developed by the Applicant and are of use in the investigation of the biological relevance of INSP171 function. These assays may address generic biological responses such as survival, proliferation and differentiation as well as specific cellular responses such as nuclear translocation of transcription factors or calcium mobilization. Such assays may, for example, focus on the three major central nervous system cell types (namely neurons, astrocytes and oligodendrocytes) as model cells for the investigation of the biological relevance of INSP171 function.

It is known in the art that purified neural cells are very difficult to culture and that neural cells grow better in mixed culture systems. However, determining the assay output for the different specific cell types within a mixed culture system is very difficult. This difficulty drastically decreases the throughput of assays employing mixed culture systems. Thus, suitable assays for the investigation of the biological relevance of INSP171 function primarily include both assays based on primary cells and assays based on cell lines. One possible way in which the biological relevance of INSP171 function can be efficiently explored in vitro is first to employ high-throughput screening assays based upon cell lines to provide an initial data set for INSP171, followed by the use of primary cultures to confirm the biological relevance predicted by the first data set.

Examples of neurobiology-related assays that have been developed by the Applicant and are suitable for further investigation of the biological relevance of INSP171 function include:

### A. Oligodendrocyte Assays:

Oligodendrocytes are responsible for myelin formation in the CNS. In multiple sclerosis they are the first cells attacked and their loss leads to major behavioral impairment. In addition to curbing inflammation, enhancing the incomplete remyelination of lesions that occurs in MS has been proposed as a therapeutic strategy for MS. Like neurons, mature oligodendrocytes do not divide but the new oligodendrocytes can arise from progenitors. There are very few of these progenitor cells in adult brain and even in embryos the number of progenitor cells is inadequate for high-throughput screening. We therefore looked for oligodendrocyte cell lines that would fulfil the following criteria: high proliferative capacity, culture conditions compatible with high-throughput screening, and possibility to induce differentiation with proteins known to act in primary oligodendrocytes.

Oli-neu is a murine cell line obtained by an immortalization of an oligodendrocyte precursor by the t-neu oncogene. They are well studied and accepted as a representative cell line to study young oligodendrocyte biology (for example, see Schuster et al., J. Neurosci. Res. 2003 Aug. 1; 73(3):324-33). Using this cell line two types of assays may be developed. The first type of assay can be used to identify factors that stimulate oligodendrocyte proliferation, and the other type can be used to identify factors promoting oligodendrocyte differentiation. Both events are key in the perspective of helping renewal and repairing demyelinating diseases.

Oli-neu are murine cells while the INSP171 protein is a Homo sapiens polypeptide. Thus, these assays may also involve a human cell line, such as MO3-13. MO3-13 results from the fusion of rabdo-myosarcoma cells with adult human oligodendrocytes (see McLaurin et al., J Neurobiol. 1995 February; 26(2):283-93). These cells have a reduced ability to differentiate into oligodendrocytes and their proliferating rate is not sufficient to allow a proliferation assay. Nevertheless, they express certain features of oligodendrocytes and their morphology is well adapted to nuclear translocation studies. The Applicant has developed assays based on nuclear translocation of three transcription factors, NF-kB, Stat-1 and Stat-2 in MO3-13 cells. The Jak/Stats transcription pathway is a complex pathway activated by many factors such as IFN α,β,γ, cytokines (for example, IL-2, IL-6 and IL-5) or hormones (for example, GH, TPO, EPO). The specificity of the response depends on the combination of activated Stats. For example, it is noticeable that INF-β activates Stat1, 2 and 3 nuclear translocations. In contrast, INF-γ activates only Stat1. In the same way, many cytokines and growth factors induce NF-kB translocation. In such assays the goal should be to get a picture of the pathways activated by the INSP171 protein. Thus, these assays provide a way of investigating whether the INSP171 polypeptide plays a role in the Jak/Stats transcription pathway. Complementary assays studying activation of other key pathways such as the PI3K, CREB and MEK pathways may be utilised to provide a full signalosome picture of INSP171.

### B. Astrocyte Assays:

The biology of astrocytes is very complex but two general states are recognised. In the ‘quiescent’ state astrocytes regulate the metabolic and excitatory level of neurons by pumping glutamate and providing energetic substratum to neurons and oligodendrocytes. In the ‘activated’ state, astrocytes produce chemokines and cytokines as well as nitric oxide. The first state can be considered as normal and healthy, while the second state is implicated in inflammation, stroke and neurodegenerative diseases. When this activated state persists it can be regarded as a pathological state.

Many factors and many pathways are known to modulate astrocyte activation. In order to identify whether INSP171 modulates astrocyte activation, assays may employ U373 cells, a human cell line of astroglioma origin. NF-kB, c-Jun as well as Stats are signalling molecules known to play pivotal roles in astrocyte activation. The Applicant has therefore developed a series of screens based on the nuclear translocation of NF-kB, c-Jun and Stat1, 2 and 3. Prototypical activators of these pathways are IL-1b, IFN-b or IFN-g. The goal in these assays is to identify whether the INSP171 proteins could be used as therapeutics themselves and to identify proteins and receptors that could be targeted for diagnostic or therapeutic applications.

### C. Neuronal Assays:

Neurons are very complex and diverse cells but they have all in common two things. First they are post-mitotic cells, and secondly they are innervating other cells. Their survival is linked to the presence of trophic factors often produced by the innervated target cells. In many neurodegenerative diseases, the loss of target innervation leads to cell body atrophy and apoptotic cell death. Therefore identification of trophic factors supplementing target deficiency is very important in treatment of neurodegenerative diseases. Accordingly, it is possible to set-up a survival assay using NS1 cells, a sub-clone of rat PC12 cells. These cells have been used for years and a lot of neurobiology knowledge has been first acquired on these cells before being confirmed on primary neurons including the pathways involved in neuron survival and differentiation (MEK, PI3K, CREB). In contrast the N2A cell line, a mouse neuroblastoma, does not respond to classical neurotrophic factors but Jun-kinase inhibitors prevent apoptosis induced by serum deprivation. Therefore, developing independent assays on these two cell lines will help to identify different types of survival-promoting proteins.

The above assays can be used to identify whether the INSP171 polypeptides promote both proliferation and differentiation or the relevant cell types. In order to identify whether the INSP171 polypeptides specifically promote neuronal differentiation, a NS1 differentiation assay based on neurite outgrowth has been developed. Promoting axonal or dendritic sprouting in neurodegenerative diseases could be advantageous for two reasons. It will first help the degenerating neurons to regrow and reestablish a contact with the target cells. Secondly, it will potentiate the so-called collateral sprouting from healthy fibers, a compensatory phenomenon that delays terminal phases of neurodegenerative diseases such as Parkinson or ALS.

### D. Endothelial Cell Assays:

The blood brain barrier (BBB) between brain and vessels is responsible for differences between cortical spinal fluid and serum compositions. The BBB results from a tight contact between endothelial cells and astrocytes. It maintains an immunotolerant status by preventing leukocytes penetration in brain, and allows the development of two parallel endocrine systems using the same intracellular signalling pathways. However, in many diseases or traumas, the BBB integrity is altered and leukocytes as well as serum proteins enter the brain inducing neuroinflammation. There is no simple in vitro model of BBB, but cultures of primary endothelial cells such as human embryonic umbilical vein endothelial cells (HUVEC) are considered to mimic some aspects of BBB biology. For example, BBB leakiness could be induced by proteins stimulating intracellular calcium release. In the perspective of identifying proteins that modulate BBB integrity, a calcium mobilization assay with or without thrombin has been developed by the Applicant using HUVEC.

A number of additional assays, employing primary cells, are also envisaged by the Applicant:

### E. Primary Oligodendrocyte Assays

One example of an assay employing primary oligodendrocytes involves the culture of oligodendrocyte progenitors from rat or mouse embryos and an analysis of the proliferation and differentiation of the oligodendrocytes. A second example of an assay employing primary oligodendrocytes involves myelin formation in mixed cortical cultures from murine embryos. These cultures contain the three major cell types of CNS as primary cells, ensuring that positive effects will be more therapeutically relevant. Finally, a model of demyelination/remyelination may also be developed in organotypic cultures of hippocampus. As noted above, as these assay models become more complex the efficiency of the assay throughput decreases dramatically and only positives from the high-throughput screening with cell lines (as described above) should be tested on these assays.

### F. Primary Astrocyte Assays

Activated astrocytes produce high levels of nitric oxide (NO). This very reactive molecule has various biological functions. Among these biological functions is killing infiltrating T-lymphocytes. However, the overproduction of NO is deleterious for neurons. During inflammation many cytokines such as IL-1β and TNF-α induce iNOS, the enzyme responsible for NO production by astrocytes. It is therefore possible to develop a screen on rat primary astrocytes to identify modulators of IL-1β-induced nitric oxide production.

### G. Primary Neuron Assays

A major problem with primary neurons is the number and the purity of the cells that can be obtained. However, there are one or two neuron subtypes, such as cortical and cerebellar granular neurons, that may prove useful for high-throughput screening. It is likely by the Applicant that a suitable assay for exploring the biological relevance of INSP171 function can be developed using rat primary cortical neurons. These assays will address the survival, differentiation and neurite outgrowth on non-permissive substratum such as myelin extract. This latter assay is directly related to CNS trauma such as spinal cord contusion, where paralysis is caused by the inability of lesioned axons to regrow through a non-permissive environment.

As neurons mature during post-natal life they can no longer be maintained in culture. Therefore an organotypique culture system was developed to assess the effect of the INSP171 polypeptides on kainate-induced excitotoxicity.

### Example 3

### Summary of Cloning of INSP171, INSP171-SV1, ISNP171-SV2, INSP171-SV3, and INSP171-SV4

INSP171 is a prediction for a reeler domain-containing secreted protein. It is a full length prediction for a 327 amino acid protein (981 bp) encoded in 5 exons. A signal peptide was predicted spanning from residues 1 to 23. Two alternative translation start sites were predicted at amino acid positions 1 and 3 of the 327 residue sequence.

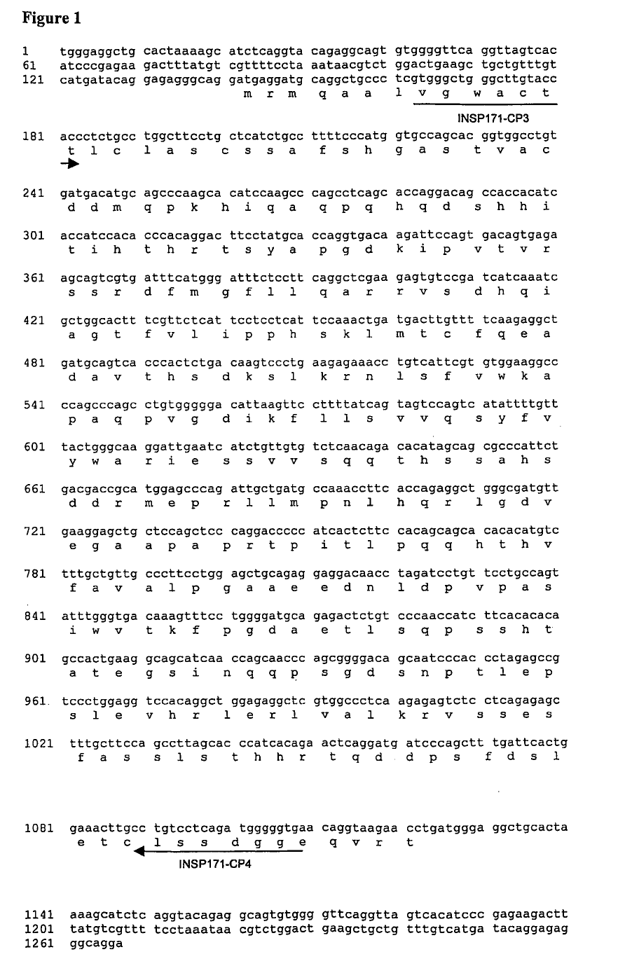

Two PCR primers (INSP171-CP3/INSP171-CP4) (FIG. 1) were designed to amplify a 947 by product. The INSP171-CP3/INSP171-CP4 PCR product would be missing the 5′-most 21 by and the 3′-most 13 by of the coding sequence.

Using the INSP171 sequence to query published EST databases identified a sequence, GenBank Accession AV698927 derived from hepatocellular carcinoma, which corresponded to a portion of the INSP171 cds. The INSP171-CP3/INSP171-CP4 primer pair was used in PCR directly on the liver tumour cDNA template and this amplification gave a product of the expected size. This product was purified from the gel, cloned and sequenced. A clone was identified which contained the expected INSP171-CP3/INSP171-CP4 PCR product sequence. This clone is pCR4-TOPO-INSP171.

Sequence analysis identified four further cloned products, all amplified from the same liver tumour cDNA template under various PCR conditions, each of which represented different variants of the INSP171 predicted ORF. These cloned products are INSP171-SV1, INSP171-SV2, INSP171-SV3 and INSP171-SV4.

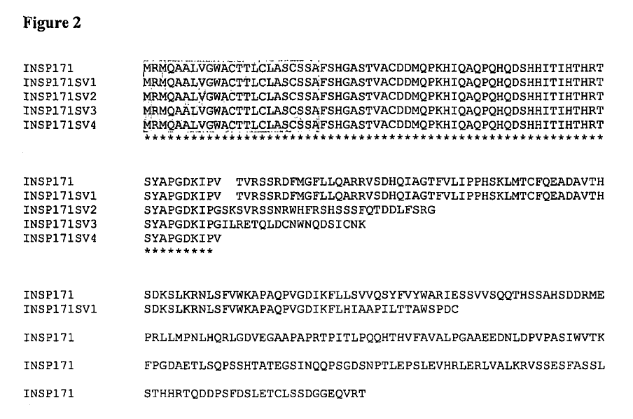

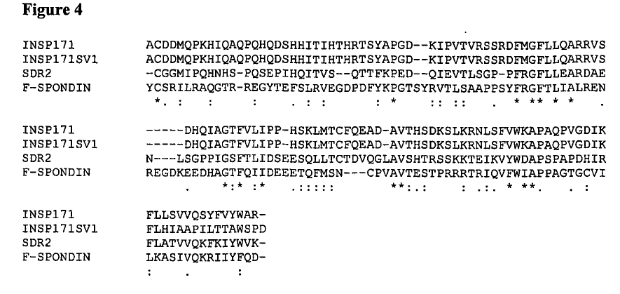

The alignment of the amino acid translations of the five cloned ORFs with the predicted INSP171 ORF is shown in FIG. 2. The amino acid sequence alignment of the reeler domain of INSP171, INSP171-SV1, human F-spondin (SwissProt Acc. No. Q9HCB6) and SDR2 (SwissProt Acc. No. P97301) is shown in FIG. 4.

cDNA cloning of INSP171 and splice variants
Preparation of Human cDNA Templates

First strand cDNA was prepared from a variety of human tissue total RNA samples (Clontech, Stratagene, Ambion, Biochain Institute and in-house preparations) using SuperScript II or SuperScript III RNase H⁻ Reverse Transcriptase (Invitrogen) according to the manufacturer's protocol.

For SuperScript II: Oligo (dT)₁₅ primer (10 at 500 μg/ml) (Promega), 2 μg human total RNA, 1 μl 10 mM dNTP mix (10 mM each of dATP, dGTP, dCTP and dTTP at neutral pH) and sterile distilled water to a final volume of 12 μl were combined in a 1.5 ml Eppendorf tube, heated to 65° C. for 5 min and chilled on ice. The contents were collected by brief centrifugation and 4 μl of 5× First-Strand Buffer, 2 μl M DTT, and 1 μl RnaseOUT™ Recombinant Ribonuclease Inhibitor (40 units/μl, Invitrogen) were added. The contents of the tube were mixed gently and incubated at 42° C. for 2 min, then 1 μl (200 units) of SuperScript II™ enzyme was added and mixed gently by pipetting. The mixture was incubated at 42° C. for 50 min and then inactivated by heating at 70° C. for 15 min. To remove RNA complementary to the cDNA, 1 μl (2 units) of E. coli RNase H (Invitrogen) was added and the reaction mixture incubated at 37° C. for 20 min.

For SuperScript III: 1 μl Oligo(dT)₂₀ primer (50 μM, Invitrogen), 2 μg human total RNA, 1 μl 10 mM dNTP mix (10 mM each of dATP, dGTP, dCTP and dTTP at neutral pH) and sterile distilled water to a final volume of 10 μl were combined in a 1.5 ml Eppendorf tube, heated to 65° C. for 5 min and then chilled on ice. For each RT reaction a cDNA synthesis mix was prepared as follows: 2 μl 10×RT buffer, 4 μl 25 mM MgCl₂, 2 μl 0.1M DTT, 1 μl RNaseOUT™ (40 U/μl) and 1 μl SuperScript III™ RT enzyme were combined in a separate tube and then 10 μl of this mix added to the tube containing the RNA/primer mixture. The contents of the tube were mixed gently, collected by brief centrifugation, and incubated at 50° C. for 50 min. The reaction was terminated by incubating at 80° C. for 5 min and the reaction mixture then chilled on ice and collected by brief centrifugation. To remove RNA complementary to the cDNA, 1 μl (2 units) of E. coli RNase H (Invitrogen) was added and the reaction mixture incubated at 37° C. for 20 min.

The final 21 μl reaction mix was diluted by adding 179 μl sterile water to give a total volume of 200 μl. This represented approximately 20 ng/μl of each individual cDNA template. The cDNA template used in the amplification INSP171 was derived from liver tumour RNA (Biochain Institute).

### Gene Specific Cloning Primers for PCR

A pair of PCR primers having a length of between 18 and 30 bases were designed to amplify the INSP171 cds using Primer Designer Software (Scientific & Educational Software, PO Box 72045, Durham, N.C. 27722-2045, USA). PCRprimers were optimized to have a Tm close to 55±10° C. and a GC content of 40-60%. Primers were selected which had high selectivity for the target sequence (INSP171) with little or no none specific priming.

PCR Amplification of INSP171 from Human cDNA Templates

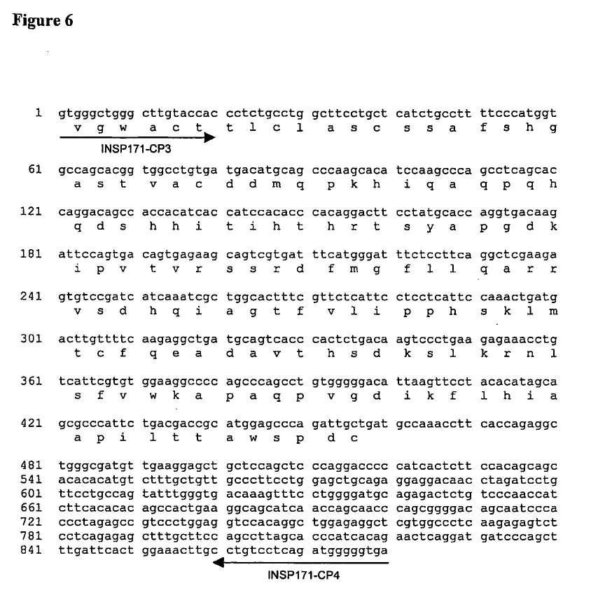

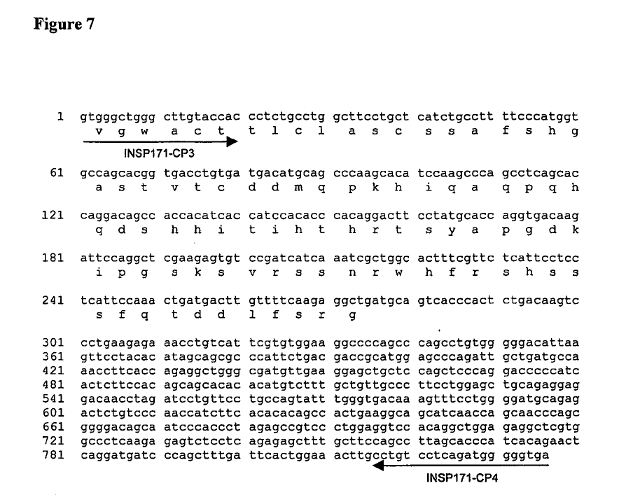

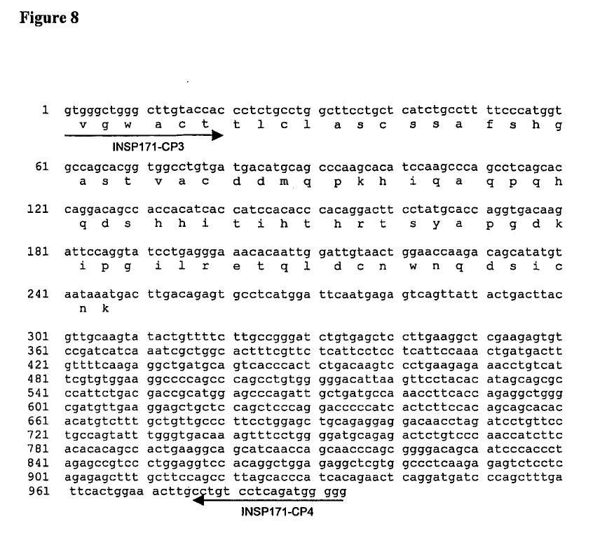

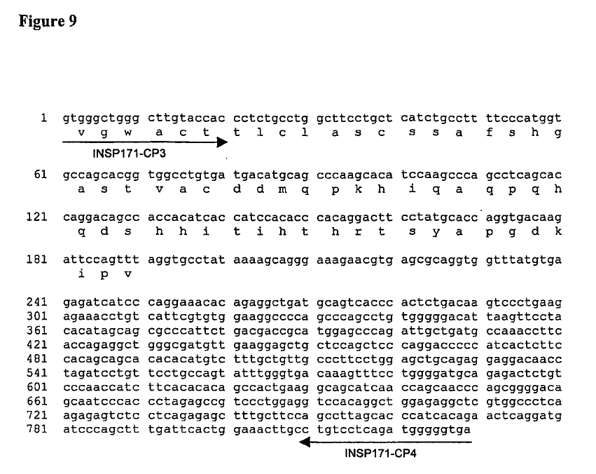

Gene-specific cloning primers (INSP171-CP3 and INSP171-CP4, Table 3, FIG. 1-12) were designed to amplify a cDNA fragment of 947 by covering the majority of the INSP171 cds. The PCR product would be missing the 5′-most 21 by and the 3′-most 13 by of the cds due to the primer positions. Interrogation of published EST databases using the INSP171 prediction identified a sequence, GenBank Accession AV698927 derived from hepatocellular carcinoma, which corresponded to a portion of the INSP171 cds. The primer pair was therefore used with a human liver tumour cDNA sample as the PCR template. PCR was performed in a final volume of 50 μl containing 1× Platinum® Taq High Fidelity (HiFi) buffer, 2 mM MgSO₄, 200 μM dNTPs, 0.2 μM of each cloning primer, 1 unit of Platinum® Taq DNA Polymerase High Fidelity (HiFi) (Invitrogen), 20 ng of liver tumour cDNA template, and either 0×, 1× or 2×PCR, Enhancer solution (Invitrogen). Cycling was performed using an MJ Research DNA Engine, programmed as follows: 94° C., 2 min; 40 cycles of 94° C., 30 sec, 61° C., 30 sec, and 68° C., 2 min; followed by 1 cycle at 68° C. for 7 min and a holding cycle at 4° C.

30 μl of each amplification product was visualized on a 0.8% agarose gel in 1×TAE buffer (Invitrogen). Products of approximately the expected molecular weight were purified from the gel using the Wizard PCR Preps DNA Purification System (Promega), eluted in 50 μl of water and subcloned directly.

### Subcloning of PCR Products

The PCR products were subcloned into the topoisomerase I modified cloning vector (pCR4-TOPO) using the TA cloning kit purchased from the Invitrogen Corporation using the conditions specified by the manufacturer. Briefly, 4 μl of gel purified PCR product was incubated for 15 min at room temperature with 1 μl of TOPO vector and 1 μl salt solution. The reaction mixture was then transformed into E. coli strain TOP10 (Invitrogen) as follows: a 50 μl aliquot of One Shot TOP10 cells was thawed on ice and 2 μl of TOPO reaction was added. The mixture was incubated for 15 min on ice and then heat shocked by incubation at 42° C. for exactly 30 s. Samples were returned to ice and 250 μl of warm (room temperature) SOC media was added. Samples were incubated with shaking (220 rpm) for 1 h at 37° C. The transformation mixture was then plated on L-broth (LB) plates containing ampicillin (100 μg/ml) and incubated overnight at 37° C.

### Plasmid DNA Preparation and Sequencing

A number of colonies were inoculated into 5 ml L-Broth (LB) containing ampicillin (100 μg/ml) and grown overnight at 37° C. with shaking at 220 rpm. Miniprep plasmid DNA was prepared from the 5 ml culture using a Biorobot 8000 robotic system (Qiagen) or Wizard Plus SV Minipreps kit (Promega cat. no. 1460) according to the manufacturer's instructions. Plasmid DNA was eluted in 80 μl of sterile water. The DNA concentration was measured using an Eppendorf BO photometer or Spectramax 190 photometer (Molecular Devices). Plasmid DNA (200-500 ng) was subjected to DNA sequencing with the T7 and T3 primers using the BigDye Terminator system (Applied Biosystems cat. no. 4390246) according to the manufacturer's instructions. The primer sequences are shown in Table 3. Sequencing reactions were purified using Dye-Ex columns (Qiagen) or Montage SEQ 96 cleanup plates (Millipore cat. no. LSKS09624) then analyzed on an Applied Biosystems 3700 sequencer.

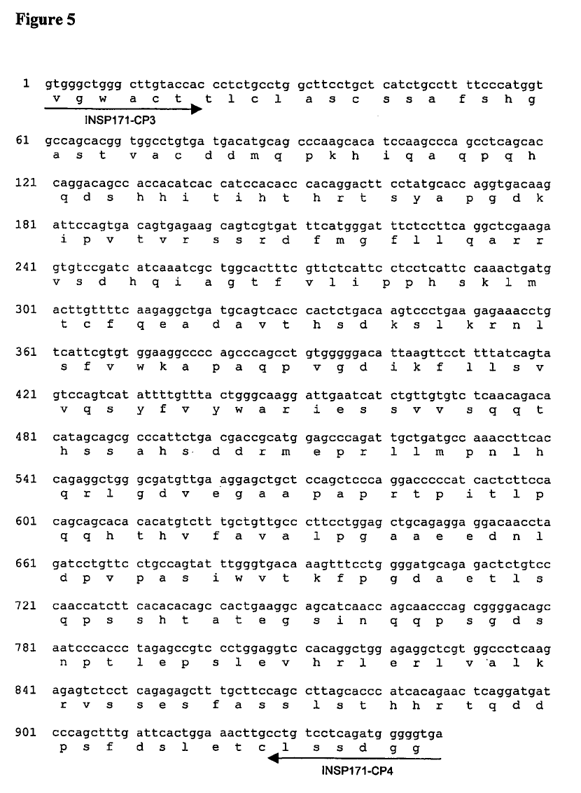

Sequence analysis identified a clone which contained the expected INSP171-CP3/INSP171-CP4 product sequence. The sequence of the cloned cDNA fragment is shown in FIG. 5. The plasmid containing the cloned PCR product is pCR4-TOPO-INSP171. Four further clones were identified each of which contained deletions at the 3′ end compared with INSP171 prediction which led to prematurely truncated ORFs in exon 4. The sequences of each of the cloned cDNA fragments (INSP171-SV1, INSP171-SV2, INSP171-SV3 and INSP171-SV4) are shown in FIGS. 5, 6, 7 and 8 respectively. The plasmid containing the cloned PCR product for INSP171SV1 is pCR4-TOPO-INSP171-SV1. The plasmid containing the cloned PCR product for INSP171SV2 is pCR4-TOPO-INSP171-SV2. The plasmid containing the cloned PCR product for INSP171SV3 is pCR4-TOPO-INSP171-SV3. The plasmid containing the cloned PCR product for INSP171SV4 is pCR4-TOPO-INSP171-SV4.

### Example 4

### Construction of Mammalian Cell Expression Vectors for INSP171, INSP171SV1, INSP171SV2, INSP171SV3 and INSP171SV4

### 4.1 Generation of Gateway Compatible INSP171 ORF Fused to an in Frame 6HIS Tag Sequence.

The first stage of the Gateway cloning process involves a two step PCR reaction which generates the ORF of INSP171 flanked at the 5′ end by an attB1 recombination site and Kozak sequence, and flanked at the 3′ end by a sequence encoding an in-frame 6 histidine (6HIS) tag, a stop codon and the attB2 recombination site (Gateway compatible cDNA). Plasmid pCR4-TOPO-INSP171 used as a template for the PCR, contained the INSP171 ORF but was missing 21 bases at the 5′ end prediction and 13 bases at the 3′ end. The missing sequence was added in PCR primers INSP171-EX1 and INSP171-EX2 (Table 3).

The first PCR reaction (in a final volume of 50 μl) contains respectively: 1 μl (30 ng) of plasmid pCR4-TOPO-INSP171, 1.5 μl dNTPs (10 mM), 10 μl of 10×Pfx polymerase buffer, 1 μl MgSO4 (50 mM), 0.5 μl each of gene specific primer (100 μM) (INSP171-EX1 and INSP171-EX2), and 0.5 μl Platinum Pfx DNA polymerase (Invitrogen). The PCR reaction was performed using an initial denaturing step of 95° C. for 2 min, followed by 12 cycles of 94° C. for 15 s; 55° C. for 30 s and 68° C. for 2 min; and a holding cycle of 4° C. The amplification product was directly purified using the Wizard PCR Preps DNA Purification System (Promega) and recovered in 50 μl sterile water according to the manufacturer's instructions.

The second PCR reaction (in a final volume of 50 μl) contained 10 μl purified PCR1 product, 1.5 μl dNTPs (10 mM), 5 μl of 10×Pfx polymerase buffer, 1 μl MgSO₄ (50 mM), 0.5 μl of each Gateway conversion primer (100 μM) (GCP forward and GCP reverse) and 0.5 μl of Platinum Pfx DNA polymerase. The conditions for the 2nd PCR reaction were: 95° C. for 1 min; 4 cycles of 94° C., 15 sec; 50° C., 30 sec and 68° C. for 2 min; 25 cycles of 94° C., 15 sec; 55° C., 30 sec and 68° C., 2 min; followed by a holding cycle of 4° C. The PCR mixture was cleaned up directly using the Wizard PCR Preps DNA Purification System (Promega) and recovered in 50 μsterile water according to the manufacturer's instructions. A 10 μl aliquot was visualized on 0.8% agarose gel in 1×TAE buffer (Invitrogen) in order to verify that the product was of the expected molecular weight (981+70=1051 bp).

4.2 Subcloning of Gateway Compatible INSP171 ORF into Gateway Entry Vector pDONR221 and Expression Vectors pEAK12d and pDEST12.2

The second stage of the Gateway cloning process involves subcloning of the Gateway modified PCR product into the Gateway entry vector pDONR221 (Invitrogen) as follows: 5 μl of purified product from PCR2 were incubated with 1.5 μl pDONR221 vector (0.1 μg/μl), 2 μl BP buffer and 1.5 μl of BP clonase enzyme mix (Invitrogen) in a final volume of 10 μl at RT for 1.5 h. The reaction was stopped by addition of 1 μl proteinase K (2 μg/μl) and incubated at 37° C. for a further 10 min. An aliquot of this reaction (2 μl) was used to transform E. coli strain TOP10 (Invitrogen) as follows: a 50 μl aliquot of One Shot TOP10 cells was thawed on ice and 2 μl of reaction mixture added. The mixture was incubated for 30 min on ice and then heat shocked by incubation at 42° C. for exactly 30 s. Samples were returned to ice and 250 μl of warm SOC media (room temperature) was added. Samples were incubated with shaking (220 rpm) for 1 h at 37° C. The transformation mixture was then plated on L-broth (LB) plates containing kanamycin (40 μg/ml) and incubated overnight at 37° C. Six of the resultant colonies were each inoculated into 1.3 ml of T-broth (TB) using a Qpix2 colony picking robot (Genetix), grown up overnight at 37° C. with shaking (220 rpm), and plasmid miniprep DNA was prepared using a Qiaprep BioRobot 8000 system (Qiagen) as described above. Plasmid DNA (150-200 ng) was subjected to DNA sequencing with 21M13 and M13Rev primers using the BigDyeTerminator system (Applied Biosystems cat. no. 4336919) according to the manufacturer's instructions. The primer sequences are shown in Table 3. Sequencing reactions were purified using Montage SEQ 96 cleanup plates (Millipore cat. no. LSKS09624) then analyzed on an Applied Biosystems 3700 sequencer.

Plasmid eluate (2 μl or approx. 150 ng) from one of the clones which contained the correct sequence (pENTR_INSP171-6HIS) was then used in a recombination reaction containing 1.5 μl of either pEAK12d vector or pDEST12.2 vector (0.1 μg/μl), 2 μl LR buffer and 1.5 μl of LR clonase (Invitrogen) in a final volume of 10 μl. The mixture was incubated at RT for 1 h, stopped by addition of proteinase K (2 μg) and incubated at 37° C. for a further 10 min. An aliquot of this reaction (2 μl) was used to transform E. coli strain TOP10 (Invitrogen) as described above. The transformation mixture was then plated on L-broth (LB) plates containing ampicillin (100 μg/ml) and incubated overnight at 37° C.

Plasmid miniprep DNA was prepared from 5 ml cultures from 6 of the resultant colonies subcloned into each vector using a Qiaprep BioRobot 8000 system (Qiagen). Plasmid DNA (200-500 ng) in the pEAK12d vector was subjected to DNA sequencing with the sequencing primers pEAK12F and pEAK12R, and gene-specific primers INSP171-SP1, INSP171-SP2 and INSP171-SP3, as described above. Plasmid DNA (200-500 ng) in the pDEST12.2 vector was subjected to DNA sequencing with the sequencing primers 21M13 and M13Rev, and gene-specific primers INSP171-SP1, INSP171-SP2 and INSP171-SP3, as described above. Primer sequences are shown in Table 3.

The plasmid of the sequence verified clone INSP171 in pEAK12d is pEAK12d_INSP171-6HIS and in pDEST12.2 is pDEST12.2_INSP171-6HIS.

4.3 Generation of Gateway compatible INSP171SV1 ORF Fused to an in Frame 6HIS Tag Sequence.

Plasmid pCR4-TOPO-INSP171-SV1 was used as PCR template to generate pEAK12d and pDEST12.2 expression clones containing the INSP171SV1 ORF sequence with a 3′ sequence encoding a 6HIS tag using the Gateway™ cloning methodology (Invitrogen) as described above. The INSP171SV1 sequence contained in plasmid pCR4-TOPO-INSP171-SV1 was missing 21 bases at the 5′ end. The missing sequence was added in PCR primer INSP171-EX1.

The first PCR reaction (in a final volume of 50 μl) contains respectively: 1 μl (30 ng) of plasmid pCR4-TOPO-INSP171-SV1, 1.5 μl dNTPs (10 mM), 10 μl of 10×Pfx polymerase buffer, 1 μl MgSO4 (50 mM), 0.5 μl each of gene specific primer (100 μM) (INSP171-EX1 and INSP171SV1-EX2), and 0.5 μl Platinum Pfx DNA polymerase (Invitrogen). The PCR reaction was performed using an initial denaturing step of 95° C. for 2 min, followed by 12 cycles of 94° C. for 15 s; 55° C. for 30 s and 68° C. for 2 min; and a holding cycle of 4° C. The amplification product was directly purified using the Wizard PCR Preps DNA Purification System (Promega) and recovered in 50 μl sterile water according to the manufacturer's instructions.

The second PCR reaction (in a final volume of 50 μl) contained 10 μl purified PCR1 product, 1.5 μl dNTPs (10 mM), 5 μl of 10×Pfx polymerase buffer, 1 μl MgSO₄ (50 mM), 0.5 μl of each Gateway conversion primer (100 μM) (GCP forward and GCP reverse) and 0.5 μl of Platinum Pfx DNA polymerase. The conditions for the 2nd PCR reaction were: 95° C. for 1 min; 4 cycles of 94° C., 15 sec; 50° C., 30 sec and 68° C. for 2 min; 25 cycles of 94° C., 15 sec; 55° C., 30 sec and 68° C., 2 min; followed by a holding cycle of 4° C. The PCR mixture was cleaned up directly using the Wizard PCR Preps DNA Purification System (Promega) and recovered in 50 μl sterile water according to the manufacturer's instructions. A 10 μl aliquot was visualized on 0.8% agarose gel in 1×TAE buffer (Invitrogen) in order to verify that the product was of the expected molecular weight (477+70=547 bp).

4.4 Subcloning of Gateway Compatible INSP171SV1 ORF into Gateway Entry Vector pDONR-Zeo and Expression Vectors pEAK12d and pDEST12.2

The second stage of the Gateway cloning process involves subcloning of the Gateway modified PCR product into the Gateway entry vector pDONR-Zeo (Invitrogen) as follows: 5 μl of cleaned product from PCR2 were incubated with 1.5 μl pDONR-Zeo vector (0.1 μm/μl), 2 μl BP buffer and 1.5 μl of BP clonase enzyme mix (Invitrogen) in a final volume of 10 μl at RT for 1 h. The reaction was stopped by addition of 1 μl proteinase K (2 μg/μl) and incubated at 37° C. for a further 10 min. An aliquot of this reaction (2 μl) was used to transform DH5α strain (Invitrogen) as follows: a 50 μl aliquot of DH5α cells was thawed on ice and 2 μl of reaction mixture added. The mixture was incubated for 30 min on ice and then heat shocked by incubation at 42° C. for exactly 30 s. Samples were returned to ice and 250 μl of warm SOC media (room temperature) was added. Samples were incubated with shaking (220 rpm) for 1 h at 37° C. The transformation mixture was then plated on L-broth (LB) plates containing kanamycin (40 μg/ml) and incubated overnight at 37° C. Plasmid mini-prep DNA was prepared from 5 ml cultures from 6 of the resultant colonies using a Qiaprep Biorobot 8000 system (Qiagen). Plasmid DNA (150-200 ng) was subjected to DNA sequencing with 21M13 and M13Rev primers using the BigDyeTerminator system (Applied Biosystems cat. no. 4390246) according to the manufacturer's instructions. The primer sequences are shown in Table 3. Sequencing reactions were purified using Montage SEQ 96 cleanup plates (Millipore cat. no. LSKS09624) then analyzed on an Applied Biosystems 3700 sequencer.

Plasmid eluate (2 μl or approx. 150 ng) from one of the clones which contained the correct sequence (pDONR-Zeo_INSP171SV1-6HIS) was then used in a recombination reaction containing 1.5 μl of either pEAK12d vector or pDEST12.2 vector (0.1 μg/μl), 2 μl LR buffer and 1.5 μl of LR clonase (Invitrogen) in a final volume of 10 μl. The mixture was incubated at RT for 1 h, stopped by addition of proteinase K (2 μg) and incubated at 37° C. for a further 10 min. An aliquot of this reaction (1 ul) was used to transform E. coli DH10B cells by electroporation as follows: a 25 μl aliquot of DH10B electrocompetent cells (Invitrogen) was thawed on ice and 1 μl of the LR reaction mix was added. The mixture was transferred to a chilled 0.1 cm electroporation cuvette and the cells electroporated using a BioRad Gene-Pulser™ according to the manufacturer's recommended protocol. SOC media (0.5 ml) which had been pre-warmed to room temperature was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing ampicillin (100 μg/ml) and incubated overnight at 37° C. Plasmid mini-prep DNA was prepared from 5 ml cultures from 4 of the resultant colonies subcloned in each vector using a Qiaprep BioRobot 8000 system (Qiagen). Plasmid DNA (200-500 ng) in the pEAK12d vector was subjected to DNA sequencing with the sequencing primers pEAK12F and pEAK12R as described above. Plasmid DNA (200-500 ng) in the pDEST12.2 vector was subjected to DNA sequencing with the sequencing primers 21M13 and M13Rev as described above.

The plasmid for one of the sequence verified clones of INSP171SV1 in pEAK12d is pEAK12d_INSP171SV1-6HIS and in pDEST12.2 is pDEST12.2_INSP171SV1-6HIS.

### 4.5 Generation of Gateway Compatible INSP171SV2 ORF Fused to an in Frame 6HIS Tag Sequence.

Plasmid pCR4-TOPO-INSP171-SV2 was used as PCR template to generate pEAK12d and pDEST12.2 expression clones containing the INSP171SV2 ORF sequence with a 3′ sequence encoding a 6HIS tag using the Gateway™ cloning methodology (Invitrogen). The INSP171SV2 sequence contained in plasmid pCR4-TOPO-INSP171-SV2 was missing 21 bases at the 5′ end. The missing sequence was added in PCR primer INSP171-EX1. INSP171SV2 also contained a single base substitution would lead to a change in the amino acid sequence of the full length protein at position 32 (A-T). Site directed mutagenesis was used to correct the mutation in the pENTR clone.

The first stage of the Gateway cloning process involves a two step PCR reaction which generates the ORF of INSP171SV2 flanked at the 5′ end by an attB1 recombination site and Kozak sequence, and flanked at the 3′ end by a sequence encoding an in-frame 6 histidine (6HIS) tag, a stop codon and the attB2 recombination site (Gateway compatible cDNA). The first PCR reaction (in a final volume of 50 μl) contains respectively: 1 μl (30 ng) of plasmid pCR4-TOPO-INSP171-SV2, 1.5 μl dNTPs (10 mM), 10 μl of 10×Pfx polymerase buffer, 1 μl MgSO4 (50 mM), 0.5 μl each of gene specific primer (100 μM) (INSP171-EX1 and INSP171SV2-EX2), and 0.5 μl Platinum Pfx DNA polymerase (Invitrogen). The PCR reaction was performed using an initial denaturing step of 95° C. for 2 min, followed by 12 cycles of 94° C. for 15 s; 55° C. for 30 s and 68° C. for 2 min; and a holding cycle of 4° C. The amplification product was directly purified using the Wizard PCR Preps DNA Purification System (Promega) and recovered in 50 μl sterile water according to the manufacturer's instructions.

The second PCR reaction (in a final volume of 50 μl) contained 10 μl purified PCR1 product, 1.5 μl dNTPs (10 mM), 5 μl of 10×Pfx polymerase buffer, 1 μl MgSO₄ (50 mM), 0.5 μl of each Gateway conversion primer (100 μM) (GCP forward and GCP reverse) and 0.5 μl of Platinum pa DNA polymerase. The conditions for the 2nd PCR reaction were: 95° C. for 1 min; 4 cycles of 94° C., 15 sec; 50° C., 30 sec and 68° C. for 2 min; 25 cycles of 94° C., 15 sec; 55° C., 30 sec and 68° C., 2 min; followed by a holding cycle of 4° C. The PCR mixture was cleaned up directly using the Wizard PCR Preps DNA Purification System (Promega) and recovered in 50 μl sterile water according to the manufacturer's instructions. A 10 μl aliquot was visualized on 0.8% agarose gel in 1×TAE buffer (Invitrogen) in order to verify that the product was of the expected molecular weight (294+70=364 bp).

4.6 Subcloning of Gateway Compatible INSP171SV2 ORF into Gateway Entry Vector pDONR-Zeo

The second stage of the Gateway cloning process involves subcloning of the Gateway modified PCR product into the Gateway entry vector pDONR-Zeo (Invitrogen) as follows: 5 μl of cleaned product from PCR2 were incubated with 1.5 μl pDONR-Zeo vector (0.1 μg/μl), 2 μl BP buffer and 1.50 of BP clonase enzyme mix (Invitrogen) in a final volume of 10 μl at RT for 1 h. The reaction was stopped by addition of 1 μl proteinase K (2 μg/μl) and incubated at 37° C. for a further 10 min. An aliquot of this reaction (2 μl) was used to transform DH5α strain (Invitrogen) as follows: a 50 μl aliquot of DH5α cells was thawed on ice and 2 μl of reaction mixture added. The mixture was incubated for 30 min on ice and then heat shocked by incubation at 42° C. for exactly 30 s. Samples were returned to ice and 250 μl of warm SOC media (room temperature) was added. Samples were incubated with shaking (220 rpm) for 1 h at 37° C. The transformation mixture was then plated on L-broth (LB) plates containing kanamycin (40 μg/ml) and incubated overnight at 37° C. Plasmid mini-prep DNA was prepared from 5 ml cultures from 6 of the resultant colonies using a Qiaprep Biorobot 8000 system (Qiagen). Plasmid DNA (150-200 ng) was subjected to DNA sequencing with 21M13 and M13Rev primers using the BigDyeTerminator system (Applied Biosystems cat. no. 4390246) according to the manufacturer's instructions. The primer sequences are shown in Table 3. Sequencing reactions were purified using Montage SEQ 96 cleanup plates (Millipore cat. no. LSKS09624) then analyzed on an Applied Biosystems 3700 sequencer. The clone which contained the correct sequence (pDONR-Zeo_INSP171SV2-6HIS) was then used for site-directed mutagenesis.

### 4.7 Site-Directed Mutagenesis (A94G) in INSP171SV2-6HIS

The INSP171SV2 sequence cloned by PCR differed from the predicted INSP171 SV2 by a single base substitution (G 94 A) which leads to the amino acid mutation A32T. This mutation was PCR induced as it was not detected in genomic DNA. In order to create a pDONR-Zeo clone containing the correct INSP171SV2 sequence, the pDONR-Zeo_INSP171-SV2-6HIS clone was used as a template for site-directed mutagenesis.

### 4.8 Gene Specific Cloning Primers for Site-Directed Mutagenesis of INSP171SV2

A pair of PCR primers, INSP171SV2 MF and INSP171SV2_MR (Table 3), was designed such that the primers annealed to opposite strands of the plasmid pDONR-Zeo_INSP171SV2-6HIS sequence and each primer annealed to 15-25 bases on either side of the amino acid to be mutated. The PCR primers were optimized to have a Tm greater than or equal to 78° C., a minimum GC content of 40%, and either a G or a C as the 3′ terminal base. Primers were designed and optimized using Primer Designer Software (Scientific & Educational Software, PO Box 72045, Durham, N.C. 27722-2045, USA).

### 4.9 Site-Directed Mutagenesis of INSP171SV2

Site-directed mutagenesis was carried out using the QuikChange® II Site-Directed Mutagenesis Kit (Stratagene) according to the manufacturer's instructions. The control reaction was performed in a final volume of 50 μl containing 1× reaction buffer, 10 ng pWhitescript 4.5 kb control plasmid, 125 ng oligonucleotide control primer #1, 125 ng control primer #2, 1 μl dNTP mix, and 2.5 units PfuUltra HF DNA polymerase. The sample reaction was performed in a final volume of 50 μl containing 1× reaction buffer, 10 ng plasmid pDONR-Zeo_INSP171SV2-6HIS template DNA, 125 ng INSP171SV2_MF primer, 125 ng INSP171SV2_MR primer, 1 μl dNTP mix, and 2.5 units PfuUltra HF DNA polymerase. Thermal cycling was performed using a MJ Research DNA Engine, programmed as follows: 95° C., 30 sec; 18 cycles of 95° C., 30 sec, 55° C., 1 min, and 68° C., 3 min 30 sec; followed by a holding cycle at 4° C.

Dpn I digestion was used to digest the methylated or hemimethylated parental DNA template (plasmid pDONR-Zeo_INSP171SV2-6HIS in the sample reaction). 1 μl of Dpn I restriction enzyme (10 U/μl, Stratagene) was added to the products of the control and sample amplification reactions. The reactions were mixed gently and incubated at 37° C. for 1 hour. Each reaction mixture was then transformed into XL1-Blue supercompetent cells (Stratagene) as follows. A 50 μl aliquot of XL1-Blue cells was thawed on ice and 1 μl of Dpn I-treated DNA was added. The mixture was incubated for 30 min on ice and then heat shocked by incubation at 42° C. for exactly 45 s. Samples were returned to ice for 2 min and 250 μl of pre-warmed (42° C.) NZY media was added. Samples were incubated with shaking (220 rpm) for 1 h at 37° C. The control transformation mixture (250 μl) was then plated on an L-broth (LB) plate containing ampicillin (100 μg/ml), X-gal (80 μg/ml), and 20 mM IPTG. The sample transformation mixture (250 μl on each of 2 plates) was plated on L-broth (LB) plates containing kanamycin (40 μg/ml). Plates were incubated overnight at 37° C.

### 4.10 Plasmid DNA Preparation and Sequencing

Eight colonies from the sample transformation plate were inoculated into 5 ml L-Broth (LB) containing kanamycin (40 μg/ml) and grown up overnight at 37° C. with shaking at 220 rpm. Plasmid mini-prep DNA was prepared using a Qiaprep Biorobot 8000 system (Qiagen). Plasmid DNA (150-200 ng) was subjected to DNA sequencing with 21M13 and M13Rev primers using the BigDyeTerminator system (Applied Biosystems cat. no. 4390246) according to the manufacturer's instructions. The primer sequences are shown in Table 3. Sequencing reactions were purified using Montage SEQ 96 cleanup plates (Millipore cat. no. LSKS09624) then analyzed on an Applied Biosystems 3700 sequencer. Sequence analysis identified a clone which contained the expected INSP171SV2 insert sequence (pDONR-Zeo_INSP171SV2-6HIS).

4.11 Subcloning of Gateway Compatible INSP171SV2 ORF into Expression Vectors pEAK12d and pDEST12.2

Plasmid DNA (2 μl or approx. 150 ng) of pDONR-Zeo_INSP171SV2-6HIS was then used in a recombination reaction containing 1.5 μl of either pEAK12d vector or pDEST12.2 vector (0.1 μg/μl), 2 μl LR buffer and 1.5 μl of LR clonase (Invitrogen) in a final volume of 10 μl. The mixture was incubated at RT for 1 h, stopped by addition of 1 μl proteinase K (2 μg/μl) and incubated at 37° C. for a further 10 min. E. coli DH10B electrocompetent cells (Invitrogen) were thawed on ice and 1 μl of the LR reaction mix was added. The mixture was transferred to a chilled 0.1 cm electroporation cuvette and the cells electroporated using a BioRad Gene-Pulser™ according to the manufacturer's recommended protocol. SOC media (0.5 ml) which had been pre-warmed to room temperature was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing ampicillin (100 μg/ml) and incubated overnight at 37° C.

Plasmid mini-prep DNA was prepared from 5 ml cultures from 4 of the resultant colonies subcloned in each vector using a Qiaprep BioRobot 8000 system (Qiagen). Plasmid DNA (200-500 ng) in the pEAK12d vector was subjected to DNA sequencing with the sequencing primers pEAK12F and pEAK12R as described above. Plasmid DNA (200-500 ng) in the pDEST12.2 vector was subjected to DNA sequencing with the sequencing primers 21M13 and M13Rev as described above.

The plasmid for one of the sequence verified clones of INSP171SV2 in pEAK12d is pEAK12d_INSP171SV2-6HIS and in pDEST12.2 is pDEST12.2_INSP171SV2-6HIS.

### 4.12 Generation of Gateway Compatible INSP171SV3 ORF Fused to an in Frame 6HIS Tag Sequence.

Plasmid pCR4-TOPO-INSP171-SV3 was used as a PCR template to generate pEAK12d and pDEST12.2 expression clones containing the INSP171SV3 ORF sequence with a 3′ sequence encoding a 6HIS tag using the Gateway™ cloning methodology (Invitrogen). The INSP171 SV3 sequence contained in plasmid pCR4-TOPO-INSP171-SV3 was missing 21 bases at the 5′ end. The 21 missing bases were added in PCR primer INSP171-EX1.

The first PCR reaction (in a final volume of 50 μl) contained 1 μl (30 ng) of plasmid pCR4-TOPO-INSP171-SV3, 1.5 μl dNTPs (10 mM), 10 μl of 10×Pfx polymerase buffer, 1 μl MgSO4 (50 mM), 0.5 each of gene specific primer (100 μM) (INSP171-EX1 and INSP171SV3-EX2), and 0.5 μl Platinum Pfx DNA polymerase (Invitrogen). The PCR reaction was performed using an initial denaturing step of 95° C. for 2 min, followed by 12 cycles of 94° C. for 15 s; 55° C. for 30 s and 68° C. for 2 min; and a holding cycle of 4° C. The amplification product was directly purified using the Wizard PCR Preps DNA Purification System (Promega) and recovered in 50 μl sterile water according to the manufacturer's instructions.

The second PCR reaction (in a final volume of 50 μl) contained 10 μl purified PCR1 product, 1.5 μl dNTPs (10 mM), 5 μl of 10×Pfx polymerase buffer, 1 μl MgSO₄ (50 mM), 0.5 μl of each Gateway conversion primer (100 μM) (GCP forward and GCP reverse) and 0.5 μl of Platinum Pfx DNA polymerase. The conditions for the 2nd PCR reaction were: 95° C. for 1 min; 4 cycles of 94° C., 15 sec; 50° C., 30 sec and 68° C. for 2 min; 25 cycles of 94° C., 15 sec; 55° C., 30 sec and 68° C., 2 min; followed by a holding cycle of 4° C. PCR product was visualized on 0.8% agarose gel in 1×TAE buffer (Invitrogen) and the band migrating at the predicted molecular mass (267+70=337 bp) was purified from gel using the Wizard SV Gel and PCR Clean-Up System (Promega Cat. # A9282) and recovered in 50 μl sterile water according to the manufacturer's instructions.

4.13 Subcloning of Gateway Compatible INSP171SV3 ORF into Gateway Entry Vector pDONR221 and Expression Vectors pEAK12d and pDEST12.2

The second stage of the Gateway cloning process involves subcloning of the Gateway modified PCR product into the Gateway entry vector pDONR221 as follows: 5 μl of purified product from PCR2 were incubated with 1.5 μl pDONR221 vector (0.1 μg/μl), 2 μl BP buffer and 1.5 μl of BP clonase enzyme mix (Invitrogen) in a final volume of 10 μl at RT for 1 h. The reaction was stopped by addition of 1 μl proteinase K (2 μg/μl) and incubated at 37° C. for a further 10 min. DH10B electrocompetent cells (25 μl) (Invitrogen) were thawed on ice and 1 μl of the BP reaction mix was added. The mixture was transferred to a chilled 0.1 cm electroporation cuvette and the cells electroporated using a BioRad Gene-Pulser™ according to the manufacturer's recommended protocol. SOC media (0.5 ml) which had been pre-warmed to room temperature was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing kanamycin (40 μg/ml) and incubated overnight at 37° C. The next day, 8 colonies were inoculated into 20 μl sterile water using a sterile toothpick. A 5 μl aliquot of the inoculum was then subjected to PCR in a total reaction volume of 20 μl containing 1× AmpliTaq® buffer, 200 μM dNTPs, 10 pmoles of 21M13 primer, 10 pmoles of M13Rev primer, 1 unit of AmpliTaq® DNA polymerase using an MJ Research DNA Engine. The cycling conditions were as follows: 94° C., 2 min; 30 cycles of 94° C., 30 sec, 55° C., 30 sec and 72° C. for 1 min. Samples were maintained at 4° C. (holding cycle) before further analysis. PCR reaction products were analyzed on 0.8% agarose gels in 1×TAE buffer. Colonies which gave the expected PCR product size (577 bp) were grown up overnight at 37° C. in 5 ml L-Broth (LB) containing kanamycin (40 μg/ml), with shaking at 220 rpm.

Plasmid eluate (2 μl or approx. 150 ng) of one PCR positive clone (pENTR_INSP171SV3-6HIS) was then used in a recombination reaction containing 1.5 μl of either pEAK12d vector or pDEST12.2 vector (0.1 μg/μl), 2 μl LR buffer and 1.5 μl of LR clonase (Invitrogen) in a final volume of 10 μl. The mixture was incubated at RT for 1 h, stopped by addition of 1 μl proteinase K (2 μg/μl) and incubated at 37° C. for a further 10 min. E. coli DH10B cells (25 μl) electrocompetent cells (Invitrogen) were thawed on ice and 1 μl of the LR reaction mix was added. The mixture was transferred to a chilled 0.1 cm electroporation cuvette and the cells electroporated using a BioRad Gene-Pulser™ according to the manufacturer's recommended protocol. SOC media (0.5 ml) which had been pre-warmed to room temperature was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing ampicillin (100 μg/ml) and incubated overnight at 37° C.

Plasmid mini-prep DNA was prepared from 5 ml cultures from 6 of the resultant colonies subcloned in each vector using a Qiaprep BioRobot 8000 system (Qiagen). Plasmid DNA (200-500 ng) in the pEAK12d vector was subjected to DNA sequencing with pEAK12F and pEAK12R primers as described above. Plasmid DNA (200-500 ng) in the pDEST12.2 vector was subjected to DNA sequencing with 21M13 and M13Rev primers as described above.

The plasmid for one of the sequence verified clones of INSP171SV3 in pEAK12d is pEAK12d_INSP171SV3-6HIS and in pDEST12.2 is pDEST12.2_INSP171SV3-6HIS.

### 4.14 Generation of Gateway Compatible INSP171SV4 ORF Fused to an in Frame 6HIS Tag Sequence.

Plasmid pCR4-TOPO-INSP171-SV4 was used as PCR template to generate pEAK12d and pDEST12.2 expression clones containing the INSP171SV4 ORF sequence with a 3′ sequence encoding a 6HIS tag using the Gateway™ cloning methodology (Invitrogen). The INSP171SV4 sequence contained in plasmid pCR4-TOPO-INSP171-SV4 was missing 21 bases at the 5′ end. The missing sequence was added in PCR primer INSP171-EX1.

The first PCR reaction (in a final volume of 50 μl) contains respectively: 1 μl (30 ng) of plasmid pCR4-TOPO-INSP171-SV4, 1.5 μl dNTPs (10 mM), 10 μl of 10×Pfx polymerase buffer, 1 μl MgSO4 (50 mM), 0.5 μl each of gene specific primer (100 μM) (INSP171-EX1 and INSP171SV4-EX2), and 0.5 μl Platinum Pfx DNA polymerase (Invitrogen). The PCR reaction was performed using an initial denaturing step of 95° C. for 2 min, followed by 12 cycles of 94° C. for 15 s; 55° C. for 30 s and 68° C. for 2 min; and a holding cycle of 4° C. The amplification product was directly purified using the Wizard PCR Preps DNA Purification System (Promega) and recovered in 50 μl sterile water according to the manufacturer's instructions.

The second PCR reaction (in a final volume of 50 μl) contained 10 μl purified PCR1 product, 1.5 μl dNTPs (10 mM), 5 μl of 10×Pfx polymerase buffer, 1 μl MgSO₄ (50 mM), 0.5 μl of each Gateway conversion primer (100 μM) (GCP forward and GCP reverse) and 0.5 μl of Platinum Pfx DNA polymerase. The conditions for the 2nd

PCR reaction were: 95° C. for 1 min; 4 cycles of 94° C., 15 sec; 50° C., 30 sec and 68° C. for 2 min; 25 cycles of 94° C., 15 sec; 55° C., 30 sec and 68° C., 2 min; followed by a holding cycle of 4° C. PCR product was visualized on 0.8% agarose gel in 1×TAE buffer (Invitrogen) and the band migrating at the predicted molecular mass (210+70=280 bp) was purified from gel using the Wizard SV Gel and PCR Clean-Up System (Promega Cat. # A9282) and recovered in 50 μl sterile water according to the manufacturer's instructions.

4.15 Subcloning of Gateway Compatible INSP171SV4 ORF into Gateway Entry Vector pDONR221 and Expression Vectors pEAK12d and pDEST12.2

The second stage of the Gateway cloning process involves subcloning of the Gateway modified PCR product into the Gateway entry vector pDONR221 as follows: 5 μl of purified product from PCR2 were incubated with 1.5 μl pDONR221 vector (0.1 μg/μl), 2 μl BP buffer and 1.5 μl of BP clonase enzyme mix (Invitrogen) in a final volume of 10 μl at RT for 1 h. The reaction was stopped by addition of 1 μl proteinase K (2 μg/μl) and incubated at 37° C. for a further 10 min. E. coli DH10B electrocompetent cells (25 μl) (Invitrogen) were thawed on ice and 1 μl of the BP reaction mix was added. The mixture was transferred to a chilled 0.1 cm electroporation cuvette and the cells electroporated using a BioRad Gene-Pulser™ according to the manufacturer's recommended protocol. SOC media (0.5 ml) which had been pre-warmed to room temperature was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing kanamycin (40 μg/ml) and incubated overnight at 37° C. The next day, 8 colonies were inoculated into 20 μl sterile water using a sterile toothpick. A 5 μl aliquot of the inoculum was then subjected to PCR in a total reaction volume of 20 μl containing 1× AmpliTaq® buffer, 200 μM dNTPs, 10 pmoles of 21M13 primer, 10 pmoles of M13Rev primer, 1 unit of AmpliTaq® DNA polymerase using an MJ Research DNA Engine. The cycling conditions were as follows: 94° C., 2 min; 30 cycles of 94° C., 30 sec, 55° C., 30 sec and 72° C. for 1 min. Samples were maintained at 4° C. (holding cycle) before further analysis. PCR reaction products were analyzed on 0.8% agarose gels in 1×TAE buffer. Colonies which gave the expected PCR product size (520 bp) were grown up overnight at 37° C. in 5 ml L-Broth (LB) containing kanamycin (40 μg/ml), with shaking at 220 rpm.

Plasmid eluate (2 μl or approx. 150 ng) from one of the positive PCR clones (pENTR_INSP171SV4-6HIS) was then used in a recombination reaction containing 1.5 μl of either pEAK12d vector or pDEST12.2 vector (0.1 μg/μl), 2 μl LR buffer and 1.5 μl of LR clonase (Invitrogen) in a final volume of 10 μl. The mixture was incubated at RT for 1 h, stopped by addition of proteinase K (2 μg) and incubated at 37° C. for a further 10 min. An aliquot of this reaction (1 ul) was used to transform E. coli DH10B cells by electroporation as follows: a 25 μl aliquot of DH10B electrocompetent cells (Invitrogen) was thawed on ice and 1 μl of the LR reaction mix was added. The mixture was transferred to a chilled 0.1 cm electroporation cuvette and the cells electroporated using a BioRad Gene-Pulser™ according to the manufacturer's recommended protocol. SOC media (0.5 ml) which had been pre-warmed to room temperature was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing ampicillin (100 μg/ml) and incubated overnight at 37° C.

Plasmid mini-prep DNA was prepared from 5 ml cultures from 6 of the resultant colonies subcloned in each vector using a Qiaprep BioRobot 8000 system (Qiagen). Plasmid DNA (200-500 ng) in the pEAK12d vector was subjected to DNA sequencing with pEAK12F and pEAK12R primers as described above. Plasmid DNA (200-500 ng) in the pDEST12.2 vector was subjected to DNA sequencing with 21M13 and M13Rev primers as described above.

The plasmid for one of the sequence verified clones of INSP171SV4 in pEAK12d is pEAK12d_INSP171SV4-6HIS and in pDEST12.2 is pDEST12.2_INSP171SV4-6HIS.

### 4.16 DNA Maxipreps

CsCl gradient purified maxi-prep DNA was prepared from 500 ml cultures of sequence verified pEAK12d clones of INSP171, INSP171SV1, INSP171SV2, INSP171SV3 and INSP171SV4 (plasmid pEAK12d_INSP171-6HIS, pEAK12d_INSP171SV1-6HIS, pEAK12d_INSP171SV2-6HIS, pEAK12d_INSP171SV3-6HIS and pEAK12d_INSP171SV4-6HIS respectively) using the method described by Sambrook J. et al., 1989 (in Molecular Cloning, a Laboratory Manual, 2^{nd} edition, Cold Spring Harbor Laboratory Press). Plasmid DNA was resuspended at a concentration of 1 μg/μl in sterile water (or 10 mM Tris-HCl pH 8.5) and stored at −20° C.

Endotoxin-free maxi-prep DNA was prepared from 500 ml cultures of sequence verified pDEST12.2 clones of INSP171, INSP171SV1, INSP171SV2, INSP171SV3 and INSP171SV4 using the EndoFree Plasmid Mega kit (Qiagen) according to the manufacturer's instructions. Purified plasmid DNA was resuspended in endotoxin free TE buffer at a final concentration of at least 3 μg/μl and stored at −20° C.

TABLE 3

| Primer | Sequence (5′-3′) |
|---|---|
| INSP171-CP3 | GTG GGC TGG GCT TGT ACC AC |
| | (SEQ ID NO: 44) |
| INSP171-CP4 | TCA CCC CCA TCT GAG GAC AGG |
| | (SEQ ID NO: 45) |
| INSP171-EX1 | GCA GGC TTC GCC ACC ATG AGG ATG CAG |
| | GCT GCC CTC GTG GGC TGG GCT TGT ACC |
| | AC |
| | (SEQ ID NO: 46) |
| INSP171-EX2 | TG ATG GTG ATG GTG GGT TCT TAC CTG |
| | TTC ACC CCC ATC TGA GGA CAG G |
| | (SEQ ID NO: 47) |
| INSP171SV1-EX2 | TG ATG GTG ATG GTG GCA ATC TGG GCT |
| | CCA TGC GGT |
| | (SEQ ID NO: 48) |
| INSP171SV2-EX2 | TG ATG GTG ATG GTG GCC TCT TGA AAA |
| | CAA GTC ATC |
| | (SEQ ID NO: 49) |
| INSP171SV3-EX2 | TG ATG GTG ATG GTG TTT ATT ACA TAT |
| | GCT GTC TTG |
| | (SEQ ID NO: 50) |
| INSP171SV4-EX2 | TG ATG GTG ATG GTG AAC TGG AAT CTT |
| | GTC ACC TGG |
| | (SEQ ID NO: 51) |
| INSP171SV2-MF | GGT GCC AGC ACG GTG GCC TGT GAT GAC |
| | ATG C |
| | (SEQ ID NO: 52) |
| INSP171SV2-MR | CAT GTC ATC ACA GGC CAC CGT GCT GGC |
| | ACC ATG |
| | (SEQ ID NO: 53) |
| INSP171-SP1 | GTG GGC TGG GCT TGT ACC AC |
| | (SEQ ID NO: 54) |
| INSP171-SP2 | ATG CCA AAC CTT CAC CAG AG |
| | (SEQ ID NO: 55) |
| INSP171-SP3 | AAA GCT GGG ATC ATC CTG AG |
| | (SEQ ID NO: 56) |
| GCP Forward | G GGG ACA AGT TTG TAC AAA AAA GCA |
| | GGC TTC GCC ACC |
| | (SEQ ID NO: 57) |
| GCP Reverse | GGG GAC CAC TTT GTA CAA GAA AGC TGG |
| | GTT TCA ATG GTG ATG GTG ATG GTG |
| | (SEQ ID NO: 58) |
| pEAK12F | GCC AGC TTG GCA CTT GAT GT |
| | (SEQ ID NO: 59) |
| pEAK12R | GAT GGA GGT GGA CGT GTC AG |
| | (SEQ ID NO: 60) |
| 21M13 | TGT AAA ACG ACG GCC AGT |
| | (SEQ ID NO: 61) |
| M13REV | CAG GAA ACA GCT ATG ACC |
| | (SEQ ID NO: 62) |
| T7 | TAA TAC GAC TCA CTA TAG G |
| | (SEQ ID NO: 63) |
| T3 | ATT AAC CCT CAC TAA AGG |
| | (SEQ ID NO: 64) |

Underlined sequence = Kozak sequence
Bold = Stop codon
Italic sequence = His tag

### Example 5

### Expression and Purification of INSP171, INSP171-SV1, INSP171-SV2, INSP171-SV3 AND INSP171-SV4

Further experiments may now be performed to determine the tissue distribution and expression levels of the INSP171, INSP171-SV1, INSP171-SV2, INSP171-SV3 AND INSP171-SV4 polypeptides in vivo, on the basis of the nucleotide and amino acid sequence disclosed herein.

The presence of the transcripts for INSP171, INSP171-SV1, INSP171-SV2, INSP171-SV3 and/or INSP171-SV4 may be investigated by PCR of cDNA from different human tissues. The INSP171, INSP171-SV1, INSP171-SV2, INSP171-SV3 and/or INSP171-SV4 transcripts may be present at very low levels in the samples tested. Therefore, extreme care is needed in the design of experiments to establish the presence of a transcript in various human tissues as a small amount of genomic contamination in the RNA preparation will provide a false positive result. Thus, all RNA should be treated with DNAse prior to use for reverse transcription. In addition, for each tissue a control reaction may be set up in which reverse transcription was not undertaken (a-ve RT control).

For example, 1 μg of total RNA from each tissue may be used to generate cDNA using Multiscript reverse transcriptase (ABI) and random hexamer primers. For each tissue, a control reaction is set up in which all the constituents are added except the reverse transcriptase eve RT control). PCR reactions are set up for each tissue on the reverse transcribed RNA samples and the minus RT controls. INSP171, INSP171-SV1, INSP171-SV2, INSP171-SV3 and/or INSP171-SV4-specific primers may readily be designed on the basis of the sequence information provided herein. The presence of a product of the correct molecular weight in the reverse transcribed sample together with the absence of a product in the minus RT control may be taken as evidence for the presence of a transcript in that tissue. Any suitable cDNA libraries may be used to screen for the INSP171, INSP171-SV1, INSP171-SV2, INSP171-SV3 and/or INSP171-SV4 transcripts, not only those generated as described above.

The tissue distribution pattern of the INSP171, INSP171-SV1, INSP171-SV2, INSP171-SV3 and/or INSP171-SV4 polypeptides will provide further useful information in relation to the function of those polypeptides.

In addition, further experiments may now be performed using expression vectors. Transfection of mammalian cell lines with these vectors may enable the high level expression of the INSP171, INSP171-SV1, INSP171-SV2, INSP171-SV3 and/or INSP171-SV4 proteins and thus enable the continued investigation of the functional characteristics of the INSP171, INSP171-SV1, INSP171-SV2, INSP171-SV3 and/or INSP171-SV4 polypeptides. The following material and methods are an example of those suitable in such experiments:

### 5.1 Cell Culture

Human Embryonic Kidney 293 cells expressing the Epstein-Barr virus Nuclear Antigen (HEK293-EBNA, Invitrogen) are maintained in suspension in Ex-cell VPRO serum-free medium (seed stock, maintenance medium, JRH). Sixteen to 20 hours prior to transfection (Day-1), cells are seeded in 2×T225 flasks (50 ml per flask in DMEM/F12 (1:1) containing 2% FBS seeding medium (JRH) at a density of 2×10⁵ cells/ml). The next day (transfection day 0) transfection takes place using the JetPEI™ reagent (2 μl/μg of plasmid DNA, PolyPlus-transfection). For each flask, plasmid DNA is co-transfected with GFP (fluorescent reporter gene) DNA. The transfection mix is then added to the 2×T225 flasks and incubated at 37° C. (5% CO₂) for 6 days. Confirmation of positive transfection may be carried out by qualitative fluorescence examination at day 1 and day 6 (Axiovert 10 Zeiss).

On day 6 (harvest day), supernatants from the two flasks are pooled and centrifuged (e.g. 4° C., 400 g) and placed into a pot bearing a unique identifier. One aliquot (500 μl) is kept for QC of the 6His-tagged protein (internal bioprocessing QC).

Scale-up batches may be produced by following the protocol called “PEI transfection of suspension cells”, referenced BP/PEI/HH/02/04, with PolyEthyleneImine from Polysciences as transfection agent.

### 5.2 Purification Process

The culture medium sample containing the recombinant protein with a C-terminal 6His tag is diluted with cold buffer A (50 mM NaH₂PO₄; 600 mM NaCl; 8.7% (w/v) glycerol, pH 7.5). The sample is filtered then through a sterile filter (Millipore) and kept at 4° C. in a sterile square media bottle (Nalgene).

The purification is performed at 4° C. on the VISION workstation (Applied Biosystems) connected to an automatic sample loader (Labomatic). The purification procedure is composed of two sequential steps, metal affinity chromatography on a Poros 20 MC (Applied Biosystems) column charged with Ni ions (4.6×50 mm, 0.83 ml), followed by gel filtration on a Sephadex G-25 medium (Amersham Pharmacia) column (1.0×10 cm).

For the first chromatography step the metal affinity column is regenerated with 30 column volumes of EDTA solution (100 mM EDTA; 1M NaCl; pH 8.0), recharged with Ni ions through washing with 15 column volumes of a 100 mM NiSO₄ solution, washed with 10 column volumes of buffer A, followed by 7 column volumes of buffer B (50 mM NaH₂PO₄; 600 mM NaCl; 8.7% (w/v) glycerol, 400 mM; imidazole, pH 7.5), and finally equilibrated with 15 column volumes of buffer A containing 15 mM imidazole. The sample is transferred, by the Labomatic sample loader, into a 200 ml sample loop and subsequently charged onto the Ni metal affinity column at a flow rate of 10 ml/min. The column is washed with 12 column volumes of buffer A, followed by 28 column volumes of buffer A containing 20 mM imidazole. During the 20 mM imidazole wash loosely attached contaminating proteins are eluted from the column. The recombinant His-tagged protein is finally eluted with 10 column volumes of buffer B at a flow rate of 2 ml/min, and the eluted protein is collected.

For the second chromatography step, the Sephadex G-25 gel-filtration column is regenerated with 2 ml of buffer D (1.137M NaCl; 2.7 mM KCl; 1.5 mM KH₂PO₄; 8 mM Na₂HPO₄; pH 7.2), and subsequently equilibrated with 4 column volumes of buffer C (137 mM NaCl; 2.7 mM KCl; 1.5 mM KH₂PO₄; 8 mM Na₂HPO₄; 20% (w/v) glycerol; pH 7.4). The peak fraction eluted from the Ni-column is automatically loaded onto the Sephadex G-25 column through the integrated sample loader on the VISION and the protein is eluted with buffer C at a flow rate of 2 ml/min. The fraction was filtered through a sterile centrifugation filter (Millipore), frozen and stored at −80° C. An aliquot of the sample is analyzed on SDS-PAGE (4-12% NuPAGE gel; Novex) Western blot with anti-His antibodies. The NuPAGE gel may be stained in a 0.1% Coomassie blue 8250 staining solution (30% methanol, 10% acetic acid) at room temperature for 1 h and subsequently destained in 20% methanol, 7.5% acetic acid until the background is clear and the protein bands clearly visible.

Following the electrophoresis the proteins are electrotransferred from the gel to a nitrocellulose membrane. The membrane is blocked with 5% milk powder in buffer E (137 mM NaCl; 2.7 mM KCl; 1.5 mM KH₂PO₄; 8 mM Na₂HPO₄; 0.1% Tween 20, pH 7.4) for 1 h at room temperature, and subsequently incubated with a mixture of 2 rabbit polyclonal anti-His antibodies (G-18 and H-15, 0.2 μg/ml each; Santa Cruz) in 2.5% milk powder in buffer E overnight at 4° C. After a further 1 hour incubation at room temperature, the membrane is washed with buffer E (3×10 min), and then incubated with a secondary HRP-conjugated anti-rabbit antibody (DAKO, HRP 0399) diluted 1/3000 in buffer E containing 2.5% milk powder for 2 hours at room temperature. After washing with buffer E (3×10 minutes), the membrane is developed with the ECL kit (Amersham Pharmacia) for 1 min. The membrane is subsequently exposed to a Hyperfilm (Amersham Pharmacia), the film developed and the western blot image visually analyzed.

For samples that showed detectable protein bands by Coomassie staining, the protein concentration may be determined using the BCA protein assay kit (Pierce) with bovine serum albumin as standard.

Furthermore, overexpression or knock-down of the expression of the polypeptides in cell lines may be used to determine the effect on transcriptional activation of the host cell genome. Dimerisation partners, co-activators and co-repressors of the INSP171, INSP171-SV1, INSP171-SV2, INSP171-SV3 and/or INSP171-SV4 polypeptides may be identified by immunoprecipitation combined with Western blotting and immunoprecipitation combined with mass spectroscopy.

1) A polypeptide, which polypeptide:
i) comprises the amino acid sequence as recited in SEQ ID NO:2, SEQ ID NO:4, SEQ ID NO:6, SEQ ID NO:8 or SEQ ID NO:10;
ii) is a fragment thereof that contains a Reeler domain and/or has an antigenic determinant in common with the polypeptides of (i); or
iii) is a functional equivalent of (i) or (ii).

2) A polypeptide which is a functional equivalent according to part (iii) of claim 1, characterised in that it is homologous to the amino acid sequence as recited in SEQ ID NO:2, SEQ ID NO:4, SEQ ID NO:6, SEQ ID NO:8 or SEQ ID NO:10.

3) A fragment or functional equivalent according to part (ii) of claim 1, which has greater than 50% sequence identity with the amino acid sequence recited SEQ ID NO:2, SEQ ID NO:4, SEQ ID NO:6, SEQ ID NO:8 or SEQ ID NO:10 or with active fragments thereof, preferably greater than 60%, 70%, 80%, 90%, 95%, 98% or 99% sequence identity.

4) A functional equivalent according to any one of the preceding claims, which exhibits significant structural homology with a polypeptide having the amino acid sequence given in SEQ ID NO:2, SEQ ID NO:4, SEQ ID NO:6, SEQ ID NO:8 or SEQ ID NO:10.

5) A fragment as recited in any one of the preceding claims, having an antigenic determinant in common with the polypeptide of part (i) of claim 1 which consists of 7 or more (for example, 8, 10, 12, 14, 16, 18, 20 or more) amino acid residues from the sequence of SEQ ID NO:2, SEQ ID NO:4, SEQ ID NO:6, SEQ ID NO:8 or SEQ ID NO:10.

6) A fusion protein comprising the polypeptide according to any one of the preceding claims.

7) The polypeptide of claim 6, wherein said polypeptide comprises a histidine tag.

8) The polypeptide of claim 7, whose sequence is recited in SEQ ID NO:12, SEQ ID NO:14, SEQ ID NO:16, SEQ ID NO:18 or SEQ ID NO:20.

9) The polypeptide of any one of the preceding claims, wherein said polypeptide comprises a signal peptide.

10) The polypeptide of claim 9, whose sequence is recited in SEQ ID NO:22, SEQ ID NO:24, SEQ ID NO:26, SEQ ID NO:28, SEQ ID NO:30 SEQ ID NO:32, SEQ ID NO:34, SEQ ID NO:36, SEQ ID NO:38 or SEQ ID NO:40.

11) A purified nucleic acid molecule which encodes a polypeptide according to any one of the preceding claims.

12) A purified nucleic acid molecule according to claim 11, which comprises or consists of the nucleic acid sequence as recited in SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37 or SEQ ID NO: 39.

13) A purified nucleic acid molecule according to claim 11 or 12 which consists of the nucleic acid sequence as recited in SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37 or SEQ ID NO: 39, or is a redundant equivalent or fragment thereof.

14) A purified nucleic acid molecule which hybridizes under high stringency conditions with a nucleic acid molecule according any one of claims 11 to 13.

15) A vector comprising a nucleic acid molecule as recited in any one of claims 11 to 13.

16) A host cell transformed with a vector according to claim 15.

17) A ligand which binds specifically to, and which preferably inhibits the activity of, a polypeptide according to any one of claims 1 to 10.

18) A ligand according to claim 17, which is an antibody.

19) A compound that either increases or decreases the level of expression or activity of a polypeptide according to any one of claims 1 to 10.

20) A compound according to claim 19 that binds to a polypeptide according to any one of claims 1 to 10 without inducing any of the biological effects of the polypeptide.

21) A compound according to claim 19 or claim 20, which is a natural or modified substrate, ligand, enzyme, receptor or structural or functional mimetic.

22) A polypeptide according to any one of claims 1 to 10, a nucleic acid molecule according to any one of claims 11 to 13, a vector according to claim 15, a host cell according to claim 16, a ligand according to claim 17 or claim 18, or a compound according to any one of claims 19 to 21, for use in therapy or diagnosis of disease.

23) A method of diagnosing a disease in a patient, comprising assessing the level of expression of a natural gene encoding a polypeptide according to any one of claims 1 to 10, or assessing the activity of a polypeptide according to any one of claims 1 to 10, in tissue from said patient and comparing said level of expression or activity to a control level, wherein a level that is different to said control level is indicative of disease.

24) A method according to claim 23 that is carried out in vitro.

25) A method according to claim 23 or claim 24, which comprises the steps of:
i) contacting a ligand according to claim 17 or claim 18 with a biological sample under conditions suitable for the formation of a ligand-polypeptide complex; and
ii) detecting said complex.

26) A method according to claim 23 or claim 24, comprising the steps of:
i) contacting a sample of tissue from the patient with a nucleic acid probe under stringent conditions that allow the formation of a hybrid complex between a nucleic acid molecule according to any one of claims 11 to 13 and the probe;
ii) contacting a control sample with said probe under the same conditions used in step i); and
iii) detecting the presence of hybrid complexes in said samples; wherein detection of levels of the hybrid complex in the patient sample that differ from levels of the hybrid complex in the control sample is indicative of disease.

27) A method according to claim 23 or claim 24, comprising:
i) contacting a sample of nucleic acid from tissue of the patient with a nucleic acid primer under stringent conditions that allow the formation of a hybrid complex between a nucleic acid molecule according to any one of claims 11 to 13 and the primer;
ii) contacting a control sample with said primer under the same conditions used in step i); and
iii) amplifying the sampled nucleic acid; and
iv) detecting the level of amplified nucleic acid from both patient and control samples; wherein detection of levels of the amplified nucleic acid in the patient sample that differ significantly from levels of the amplified nucleic acid in the control sample is indicative of disease.

28) A method according to claim 23 or claim 24 comprising:
i) obtaining a tissue sample from a patient being tested for disease;
ii) isolating a nucleic acid molecule according to any one of claims 11 to 13 from said tissue sample; and
iii) diagnosing the patient for disease by detecting the presence of a mutation which is associated with disease in the nucleic acid molecule as an indication of the disease.

29) The method of claim 28, further comprising amplifying the nucleic acid molecule to form an amplified product and detecting the presence or absence of a mutation in the amplified product.

30) The method of either claim 28 or 29, wherein the presence or absence of the mutation in the patient is detected by contacting said nucleic acid molecule with a nucleic acid probe that hybridises to said nucleic acid molecule under stringent conditions to form a hybrid double-stranded molecule, the hybrid double-stranded molecule having an unhybridized portion of the nucleic acid probe strand at any portion corresponding to a mutation associated with disease; and detecting the presence or absence of an unhybridized portion of the probe strand as an indication of the presence or absence of a disease-associated mutation.

31) A method according to any one of claims 23 to 30, wherein said disease is selected from neoplasm, cancer, brain tumour, glioma, bone tumor, lung tumor, breast tumour, prostate tumour, colon tumour, hepatocellular carcinoma or liver tumour, endometrial carcinoma, hemangioma, myeloproliferative disorder, leukemia, hematological disease, neutropenia, thrombocytopenia, angiogenesis disorders, dermatological disease, aging, wounds, burns, fibrosis, cardiovascular disease, restenosis, heart disease, peripheral vascular disease, coronary artery disease, oedema, thromboembolism, dysmenorrhea, endometriosis, pre-eclampsia, lung disease, COPD, asthma bone disease, renal disease, glomerulonephritis, liver disease, Crohn's disease, gastritis, ulcerative colitis, ulcer, immune disorder, autoimmune disease, arthritis, rheumatoid arthritis, psoriasis, epidermolysis bullosa, systemic lupus erythematosus, ankylosing spondylitis, Lyme disease, multiple sclerosis, neurodegeneration, stroke, brain/spinal cord injury, Alzheimer's disease, Parkinson's disease, motor neurone disease, neuromuscular disease, CNS inflammation, cerebellar degeneration, amylotrophic lateral sclerosis, head injury damage, and other neurological abnormalities, HIV, AIDS, cytomegalovirus infection and fungal infection.

32) A method according to any one of claims 23 to 30, wherein said disease is selected from neurological disorders such as, for example, schizophrenia, neurodegeneration, neurodevelopmental disease, stroke, brain/spinal cord injury, Alzheimer's disease, Parkinson's disease, motor neurone disease, neuromuscular disease, CNS inflammation, cerebellar degeneration, amylotrophic lateral sclerosis, head injury damage, and other neurological abnormalities, including Autosomal recessive lissencephaly with cerebellar hypoplasia and autistic disorders.

33) Use of a polypeptide according to any one of claims 1 to 10 as a Reeler domain containing protein.

34) A pharmaceutical composition comprising a polypeptide according to any one of claims 1 to 10, a nucleic acid molecule according to any one of claims 11 to 13, a vector according to claim 15, a host cell according to claim 16, a ligand according to claim 17 or 15, or a compound according to any one of claims 19 to 21.

35) A vaccine composition comprising a polypeptide according to any one of claims 1 to 10 or a nucleic acid molecule according to any one of claims 11 to 13.

36) A polypeptide according to any one of claims 1 to 10, a nucleic acid molecule according to any one of claims 11 to 13, a vector according to claim 15, a host cell according to claim 16, a ligand according to claim 17 or 15, a compound according to any one of claims 19 to 21, or a pharmaceutical composition according to claim 34, for use in the manufacture of a medicament for the treatment of a disease selected from neoplasm, cancer, brain tumour, glioma, bone tumor, lung tumor, breast tumour, prostate tumour, colon tumour, hepatocellular carcinoma or liver tumour, endometrial carcinoma, hemangioma, myeloproliferative disorder, leukemia, hematological disease, neutropenia, thrombocytopenia, angiogenesis disorders, dermatological disease, aging, wounds, burns, fibrosis, cardiovascular disease, restenosis, heart disease, peripheral vascular disease, coronary artery disease, oedema, thromboembolism, dysmenorrhea, endometriosis, pre-eclampsia, lung disease, COPD, asthma bone disease, renal disease, glomerulonephritis, liver disease, Crohn's disease, gastritis, ulcerative colitis, ulcer, immune disorder, autoimmune disease, arthritis, rheumatoid arthritis, psoriasis, epidermolysis bullosa, systemic lupus erythematosus, ankylosing spondylitis, Lyme disease, multiple sclerosis, neurodegeneration, stroke, brain/spinal cord injury, Alzheimer's disease, Parkinson's disease, motor neurone disease, neuromuscular disease, CNS inflammation, cerebellar degeneration, amylotrophic lateral sclerosis, head injury damage, and other neurological abnormalities, HIV, AIDS, cytomegalovirus infection, and fungal infection.

37) Use of a polypeptide according to any one of claims 1 to 10, a nucleic acid molecule according to any one of claims 11 to 13, a vector according to claim 15, a host cell according to claim 16, a ligand according to claim 17 or 15, a compound according to any one of claims 19 to 21, or a pharmaceutical composition according to claim 34, in the manufacture of a medicament for the treatment of a disease selected from neurological disorders such as, for example, schizophrenia, neurodegeneration, neurodevelopmental disease, stroke, brain/spinal cord injury, Alzheimer's disease, Parkinson's disease, motor neurone disease, neuromuscular disease, CNS inflammation, cerebellar degeneration, amylotrophic lateral sclerosis, head injury damage, and other neurological abnormalities, including Autosomal recessive lissencephaly with cerebellar hypoplasia and autistic disorders.

38) A method of treating a disease in a patient, comprising administering to the patient a polypeptide according to any one of claims 1 to 10, a nucleic acid molecule according to any one of claims 11 to 13, a vector according to claim 15, a host cell according to claim 16, a ligand according to claim 17 or 15, or a compound according to any one of claims 19 to 21, or a pharmaceutical composition according to claim 34.

39) A method according to claim 38, wherein, for diseases in which the expression of the natural gene or the activity of the polypeptide is lower in a diseased patient when compared to the level of expression or activity in a healthy patient, the polypeptide, nucleic acid molecule, vector, ligand, compound or composition administered to the patient is an agonist.

40) A method according to claim 38, wherein, for diseases in which the expression of the natural gene or activity of the polypeptide is higher in a diseased patient when compared to the level of expression or activity in a healthy patient, the polypeptide, nucleic acid molecule, vector, ligand, compound or composition administered to the patient is an antagonist.

41) A method of monitoring the therapeutic treatment of disease in a patient, comprising monitoring over a period of time the level of expression or activity of a polypeptide according to any one of claims 1 to 10, or the level of expression of a nucleic acid molecule according to any one of claims 11 to 13 in tissue from said patient, wherein altering said level of expression or activity over the period of time towards a control level is indicative of regression of said disease.

42) A method for the identification of a compound that is effective in the treatment and/or diagnosis of disease, comprising contacting a polypeptide according to any one of claims 1 to 10, or a nucleic acid molecule according to any one of claims 11 to 13 with one or more compounds suspected of possessing binding affinity for said polypeptide or nucleic acid molecule, and selecting a compound that binds specifically to said nucleic acid molecule or polypeptide.

43) A kit useful for diagnosing disease comprising a first container containing a nucleic acid probe that hybridises under stringent conditions with a nucleic acid molecule according to any one of claims 11 to 13; a second container containing primers useful for amplifying said nucleic acid molecule; and instructions for using the probe and primers for facilitating the diagnosis of disease.

44) The kit of claim 43, further comprising a third container holding an agent for digesting unhybridized RNA.

45) A kit comprising an array of nucleic acid molecules, at least one of which is a nucleic acid molecule according to any one of claims 11 to 13.

46) A kit comprising one or more antibodies that bind to a polypeptide as recited in any one of claims 1 to 10; and a reagent useful for the detection of a binding reaction between said antibody and said polypeptide.

47) A transgenic or knockout non-human animal that has been transformed to express higher, lower or absent levels of a polypeptide according to any one of claims 1 to 10.

48) A method for screening for a compound effective to treat disease, by contacting a non-human transgenic animal according to claim 47 with a candidate compound and determining the effect of the compound on the disease of the animal.

49) A method according to claims 38-44 or claim 48, wherein said disease is one of the diseases set forth in claim 31 or claim 32.

50) The use of an INSP171 polypeptide as a target for screening candidate drugs for treating or preventing a Reeler domain containing protein related disorder.

51) Method of selecting biologically active compounds comprising:
(i) contacting a candidate compound with recombinant host cells expressing an INSP171 polypeptide;
(ii) selecting compounds that bind said INSP171 polypeptide at the surface of said cells and/or that modulate the activity of the INSP171 polypeptide.


SEQUENCE LISTING (74 sequences)

SEQ ID NO: 1 - DNA, 915 bp, Homo sapiens
ttttcccatggtgccagcacggtggcctgtgatgacatgcagcccaagcacatccaagcccagcctcagcaccaggacagccaccacatcaccatccacacccacaggacttcctatgcaccaggtgacaagattccagtgacagtgagaagcagtcgtgatttcatgggatttctccttcaggctcgaagagtgtccgatcatcaaatcgctggcactttcgttctcattcctcctcattccaaactgatgacttgttttcaagaggctgatgcagtcacccactctgacaagtccctgaagagaaacctgtcattcgtgtggaaggccccagcccagcctgtgggggacattaagttccttttatcagtagtccagtcatattttgtttactgggcaaggattgaatcatctgttgtgtctcaacagacacatagcagcgcccattctgacgaccgcatggagcccagattgctgatgccaaaccttcaccagaggctgggcgatgttgaaggagctgctccagctcccaggacccccatcactcttccacagcagcacacacatgtctttgctgttgcccttcctggagctgcagaggaggacaacctagatcctgttcctgccagtatttgggtgacaaagtttcctggggatgcagagactctgtcccaaccatcttcacacacagccactgaaggcagcatcaaccagcaacccagcggggacagcaatcccaccctagagccgtccctggaggtccacaggctggagaggctcgtggccctcaagagagtctcctcagagagctttgcttccagccttagcacccatcacagaactcaggatgatcccagctttgattcactggaaacttgcctgtcctcagatgggggtgaacaggtaagaacctga

SEQ ID NO: 2 - protein, 304 aa, Homo sapiens
FSHGASTVACDDMQPKHIQAQPQHQDSHHITIHTHRTSYAPGDKIPVTVRSSRDFMGFLLQARRVSDHQIAGTFVLIPPHSKLMTCFQEADAVTHSDKSLKRNLSFVWKAPAQPVGDIKFLLSVVQSYFVYWARIESSVVSQQTHSSAHSDDRMEPRLLMPNLHQRLGDVEGAAPAPRTPITLPQQHTHVFAVALPGAAEEDNLDPVPASIWVTKFPGDAETLSQPSSHTATEGSINQQPSGDSNPTLEPSLEVHRLERLVALKRVSSESFASSLSTHHRTQDDPSFDSLETCLSSDGGEQVRT

SEQ ID NO: 3 - DNA, 408 bp, Homo sapiens
ttttcccatggtgccagcacggtggcctgtgatgacatgcagcccaagcacatccaagcccagcctcagcaccaggacagccaccacatcaccatccacacccacaggacttcctatgcaccaggtgacaagattccagtgacagtgagaagcagtcgtgatttcatgggatttctccttcaggctcgaagagtgtccgatcatcaaatcgctggcactttcgttctcattcctcctcattccaaactgatgacttgttttcaagaggctgatgcagtcacccactctgacaagtccctgaagagaaacctgtcattcgtgtggaaggccccagcccagcctgtgggggacattaagttcctacacatagcagcgcccattctgacgaccgcatggagcccagattgc

SEQ ID NO: 4 - protein, 136 aa, Homo sapiens
FSHGASTVACDDMQPKHIQAQPQHQDSHHITIHTHRTSYAPGDKIPVTVRSSRDFMGFLLQARRVSDHQIAGTFVLIPPHSKLMTCFQEADAVTHSDKSLKRNLSFVWKAPAQPVGDIKFLHIAAPILTTAWSPDC

SEQ ID NO: 5 - DNA, 225 bp, Homo sapiens
ttttcccatggtgccagcacggtggcctgtgatgacatgcagcccaagcacatccaagcccagcctcagcaccaggacagccaccacatcaccatccacacccacaggacttcctatgcaccaggtgacaagattccaggctcgaagagtgtccgatcatcaaatcgctggcactttcgttctcattcctcctcattccaaactgatgacttgttttcaagaggc

SEQ ID NO: 6 - protein, 74 aa, Homo sapiens
FSHGASTVACDDMQPKHIQAQPQHQDSHHITIHTHRTSYAPGDKIPGSKSVRSSNRWHFRSHSSSFQTDDLFSR

SEQ ID NO: 7 - DNA, 198 bp, Homo sapiens
ttttcccatggtgccagcacggtggcctgtgatgacatgcagcccaagcacatccaagcccagcctcagcaccaggacagccaccacatcaccatccacacccacaggacttcctatgcaccaggtgacaagattccaggtatcctgagggaaacacaattggattgtaactggaaccaagacagcatatgtaataaa

SEQ ID NO: 8 - protein, 66 aa, Homo sapiens
FSHGASTVACDDMQPKHIQAQPQHQDSHHITIHTHRTSYAPGDKIPGILRETQLDCNWNQDSICNK

SEQ ID NO: 9 - DNA, 141 bp, Homo sapiens
ttttcccatggtgccagcacggtggcctgtgatgacatgcagcccaagcacatccaagcccagcctcagcaccaggacagccaccacatcaccatccacacccacaggacttcctatgcaccaggtgacaagattccagtt

SEQ ID NO: 10 - protein, 47 aa, Homo sapiens
FSHGASTVACDDMQPKHIQAQPQHQDSHHITIHTHRTSYAPGDKIPV

SEQ ID NO: 11 - DNA, 933 bp, Artificial Sequence
  INSP171 his tag mature polynucleotide
ttttcccatggtgccagcacggtggcctgtgatgacatgcagcccaagcacatccaagcccagcctcagcaccaggacagccaccacatcaccatccacacccacaggacttcctatgcaccaggtgacaagattccagtgacagtgagaagcagtcgtgatttcatgggatttctccttcaggctcgaagagtgtccgatcatcaaatcgctggcactttcgttctcattcctcctcattccaaactgatgacttgttttcaagaggctgatgcagtcacccactctgacaagtccctgaagagaaacctgtcattcgtgtggaaggccccagcccagcctgtgggggacattaagttccttttatcagtagtccagtcatattttgtttactgggcaaggattgaatcatctgttgtgtctcaacagacacatagcagcgcccattctgacgaccgcatggagcccagattgctgatgccaaaccttcaccagaggctgggcgatgttgaaggagctgctccagctcccaggacccccatcactcttccacagcagcacacacatgtctttgctgttgcccttcctggagctgcagaggaggacaacctagatcctgttcctgccagtatttgggtgacaaagtttcctggggatgcagagactctgtcccaaccatcttcacacacagccactgaaggcagcatcaaccagcaacccagcggggacagcaatcccaccctagagccgtccctggaggtccacaggctggagaggctcgtggccctcaagagagtctcctcagagagctttgcttccagccttagcacccatcacagaactcaggatgatcccagctttgattcactggaaacttgcctgtcctcagatgggggtgaacaggtaagaacctgacaccatcaccatcaccat

SEQ ID NO: 12 - protein, 310 aa, Artificial Sequence
  INSP171 his tag mature polypeptide
FSHGASTVACDDMQPKHIQAQPQHQDSHHITIHTHRTSYAPGDKIPVTVRSSRDFMGFLLQARRVSDHQIAGTFVLIPPHSKLMTCFQEADAVTHSDKSLKRNLSFVWKAPAQPVGDIKFLLSVVQSYFVYWARIESSVVSQQTHSSAHSDDRMEPRLLMPNLHQRLGDVEGAAPAPRTPITLPQQHTHVFAVALPGAAEEDNLDPVPASIWVTKFPGDAETLSQPSSHTATEGSINQQPSGDSNPTLEPSLEVHRLERLVALKRVSSESFASSLSTHHRTQDDPSFDSLETCLSSDGGEQVRTHHHHHH

SEQ ID NO: 13 - DNA, 426 bp, Artificial Sequence
  INSP171-SV1 his tag mature polynucleotide
ttttcccatggtgccagcacggtggcctgtgatgacatgcagcccaagcacatccaagcccagcctcagcaccaggacagccaccacatcaccatccacacccacaggacttcctatgcaccaggtgacaagattccagtgacagtgagaagcagtcgtgatttcatgggatttctccttcaggctcgaagagtgtccgatcatcaaatcgctggcactttcgttctcattcctcctcattccaaactgatgacttgttttcaagaggctgatgcagtcacccactctgacaagtccctgaagagaaacctgtcattcgtgtggaaggccccagcccagcctgtgggggacattaagttcctacacatagcagcgcccattctgacgaccgcatggagcccagattgccaccatcaccatcaccat

SEQ ID NO: 14 - protein, 142 aa, Artificial Sequence
  INSP171-SV1 his tag mature polypeptide
FSHGASTVACDDMQPKHIQAQPQHQDSHHITIHTHRTSYAPGDKIPVTVRSSRDFMGFLLQARRVSDHQIAGTFVLIPPHSKLMTCFQEADAVTHSDKSLKRNLSFVWKAPAQPVGDIKFLHIAAPILTTAWSPDCHHHHHH

SEQ ID NO: 15 - DNA, 243 bp, Artificial Sequence
  INSP171-SV2 his tag mature polynucleotide
ttttcccatggtgccagcacggtggcctgtgatgacatgcagcccaagcacatccaagcccagcctcagcaccaggacagccaccacatcaccatccacacccacaggacttcctatgcaccaggtgacaagattccaggctcgaagagtgtccgatcatcaaatcgctggcactttcgttctcattcctcctcattccaaactgatgacttgttttcaagaggccaccatcaccatcaccat

SEQ ID NO: 16 - protein, 80 aa, Artificial Sequence
  INSP171-SV2 his tag mature polypeptide
FSHGASTVACDDMQPKHIQAQPQHQDSHHITIHTHRTSYAPGDKIPGSKSVRSSNRWHFRSHSSSFQTDDLFSRHHHHHH

SEQ ID NO: 17 - DNA, 216 bp, Artificial Sequence
  INSP171-SV3 his tag mature polynucleotide
ttttcccatggtgccagcacggtggcctgtgatgacatgcagcccaagcacatccaagcccagcctcagcaccaggacagccaccacatcaccatccacacccacaggacttcctatgcaccaggtgacaagattccaggtatcctgagggaaacacaattggattgtaactggaaccaagacagcatatgtaataaacaccatcaccatcaccat

SEQ ID NO: 18 - protein, 72 aa, Artificial Sequence
  INSP171-SV3 his tag mature polypeptide
FSHGASTVACDDMQPKHIQAQPQHQDSHHITIHTHRTSYAPGDKIPGILRETQLDCNWNQDSICNKHHHHHH

SEQ ID NO: 19 - DNA, 159 bp, Artificial Sequence
  INSP171-SV4 his tag mature polynucleotide
ttttcccatggtgccagcacggtggcctgtgatgacatgcagcccaagcacatccaagcccagcctcagcaccaggacagccaccacatcaccatccacacccacaggacttcctatgcaccaggtgacaagattccagttcaccatcaccatcaccat

SEQ ID NO: 20 - protein, 53 aa, Artificial Sequence
  INSP171-SV4 his tag mature polypeptide
FSHGASTVACDDMQPKHIQAQPQHQDSHHITIHTHRTSYAPGDKIPVHHHHHH

SEQ ID NO: 21 - DNA, 984 bp, Homo sapiens
atgaggatgcaggctgccctcgtgggctgggcttgtaccaccctctgcctggcttcctgctcatctgccttttcccatggtgccagcacggtggcctgtgatgacatgcagcccaagcacatccaagcccagcctcagcaccaggacagccaccacatcaccatccacacccacaggacttcctatgcaccaggtgacaagattccagtgacagtgagaagcagtcgtgatttcatgggatttctccttcaggctcgaagagtgtccgatcatcaaatcgctggcactttcgttctcattcctcctcattccaaactgatgacttgttttcaagaggctgatgcagtcacccactctgacaagtccctgaagagaaacctgtcattcgtgtggaaggccccagcccagcctgtgggggacattaagttccttttatcagtagtccagtcatattttgtttactgggcaaggattgaatcatctgttgtgtctcaacagacacatagcagcgcccattctgacgaccgcatggagcccagattgctgatgccaaaccttcaccagaggctgggcgatgttgaaggagctgctccagctcccaggacccccatcactcttccacagcagcacacacatgtctttgctgttgcccttcctggagctgcagaggaggacaacctagatcctgttcctgccagtatttgggtgacaaagtttcctggggatgcagagactctgtcccaaccatcttcacacacagccactgaaggcagcatcaaccagcaacccagcggggacagcaatcccaccctagagccgtccctggaggtccacaggctggagaggctcgtggccctcaagagagtctcctcagagagctttgcttccagccttagcacccatcacagaactcaggatgatcccagctttgattcactggaaacttgcctgtcctcagatgggggtgaacaggtaagaacctga

SEQ ID NO: 22 - protein, 327 aa, Homo sapiens
MRMQAALVGWACTTLCLASCSSAFSHGASTVACDDMQPKHIQAQPQHQDSHHITIHTHRTSYAPGDKIPVTVRSSRDFMGFLLQARRVSDHQIAGTFVLIPPHSKLMTCFQEADAVTHSDKSLKRNLSFVWKAPAQPVGDIKFLLSVVQSYFVYWARIESSVVSQQTHSSAHSDDRMEPRLLMPNLHQRLGDVEGAAPAPRTPITLPQQHTHVFAVALPGAAEEDNLDPVPASIWVTKFPGDAETLSQPSSHTATEGSINQQPSGDSNPTLEPSLEVHRLERLVALKRVSSESFASSLSTHHRTQDDPSFDSLETCLSSDGGEQVRT

SEQ ID NO: 23 - DNA, 477 bp, Homo sapiens
atgaggatgcaggctgccctcgtgggctgggcttgtaccaccctctgcctggcttcctgctcatctgccttttcccatggtgccagcacggtggcctgtgatgacatgcagcccaagcacatccaagcccagcctcagcaccaggacagccaccacatcaccatccacacccacaggacttcctatgcaccaggtgacaagattccagtgacagtgagaagcagtcgtgatttcatgggatttctccttcaggctcgaagagtgtccgatcatcaaatcgctggcactttcgttctcattcctcctcattccaaactgatgacttgttttcaagaggctgatgcagtcacccactctgacaagtccctgaagagaaacctgtcattcgtgtggaaggccccagcccagcctgtgggggacattaagttcctacacatagcagcgcccattctgacgaccgcatggagcccagattgc

SEQ ID NO: 24 - protein, 159 aa, Homo sapiens
MRMQAALVGWACTTLCLASCSSAFSHGASTVACDDMQPKHIQAQPQHQDSHHITIHTHRTSYAPGDKIPVTVRSSRDFMGFLLQARRVSDHQIAGTFVLIPPHSKLMTCFQEADAVTHSDKSLKRNLSFVWKAPAQPVGDIKFLHIAAPILTTAWSPDC

SEQ ID NO: 25 - DNA, 294 bp, Homo sapiens
atgaggatgcaggctgccctcgtgggctgggcttgtaccaccctctgcctggcttcctgctcatctgccttttcccatggtgccagcacggtggcctgtgatgacatgcagcccaagcacatccaagcccagcctcagcaccaggacagccaccacatcaccatccacacccacaggacttcctatgcaccaggtgacaagattccaggctcgaagagtgtccgatcatcaaatcgctggcactttcgttctcattcctcctcattccaaactgatgacttgttttcaagaggc

SEQ ID NO: 26 - protein, 98 aa, Homo sapiens
MRMQAALVGWACTTLCLASCSSAFSHGASTVACDDMQPKHIQAQPQHQDSHHITIHTHRTSYAPGDKIPGSKSVRSSNRWHFRSHSSSFQTDDLFSRG

SEQ ID NO: 27 - DNA, 267 bp, Homo sapiens
atgaggatgcaggctgccctcgtgggctgggcttgtaccaccctctgcctggcttcctgctcatctgccttttcccatggtgccagcacggtggcctgtgatgacatgcagcccaagcacatccaagcccagcctcagcaccaggacagccaccacatcaccatccacacccacaggacttcctatgcaccaggtgacaagattccaggtatcctgagggaaacacaattggattgtaactggaaccaagacagcatatgtaataaa

SEQ ID NO: 28 - protein, 89 aa, Homo sapiens
MRMQAALVGWACTTLCLASCSSAFSHGASTVACDDMQPKHIQAQPQHQDSHHITIHTHRTSYAPGDKIPGILRETQLDCNWNQDSICNK

SEQ ID NO: 29 - DNA, 210 bp, Homo sapiens
atgaggatgcaggctgccctcgtgggctgggcttgtaccaccctctgcctggcttcctgctcatctgccttttcccatggtgccagcacggtggcctgtgatgacatgcagcccaagcacatccaagcccagcctcagcaccaggacagccaccacatcaccatccacacccacaggacttcctatgcaccaggtgacaagattccagtt

SEQ ID NO: 30 - protein, 70 aa, Homo sapiens
MRMQAALVGWACTTLCLASCSSAFSHGASTVACDDMQPKHIQAQPQHQDSHHITIHTHRTSYAPGDKIPV

SEQ ID NO: 31 - DNA, 1002 bp, Artificial Sequence
  INSP171 his tag Full length polynucleotide
atgaggatgcaggctgccctcgtgggctgggcttgtaccaccctctgcctggcttcctgctcatctgccttttcccatggtgccagcacggtggcctgtgatgacatgcagcccaagcacatccaagcccagcctcagcaccaggacagccaccacatcaccatccacacccacaggacttcctatgcaccaggtgacaagattccagtgacagtgagaagcagtcgtgatttcatgggatttctccttcaggctcgaagagtgtccgatcatcaaatcgctggcactttcgttctcattcctcctcattccaaactgatgacttgttttcaagaggctgatgcagtcacccactctgacaagtccctgaagagaaacctgtcattcgtgtggaaggccccagcccagcctgtgggggacattaagttccttttatcagtagtccagtcatattttgtttactgggcaaggattgaatcatctgttgtgtctcaacagacacatagcagcgcccattctgacgaccgcatggagcccagattgctgatgccaaaccttcaccagaggctgggcgatgttgaaggagctgctccagctcccaggacccccatcactcttccacagcagcacacacatgtctttgctgttgcccttcctggagctgcagaggaggacaacctagatcctgttcctgccagtatttgggtgacaaagtttcctggggatgcagagactctgtcccaaccatcttcacacacagccactgaaggcagcatcaaccagcaacccagcggggacagcaatcccaccctagagccgtccctggaggtccacaggctggagaggctcgtggccctcaagagagtctcctcagagagctttgcttccagccttagcacccatcacagaactcaggatgatcccagctttgattcactggaaacttgcctgtcctcagatgggggtgaacaggtaagaacctgacaccatcaccatcaccat

SEQ ID NO: 32 - protein, 333 aa, Artificial Sequence
  INSP171 his tag Full length polypeptide
MRMQAALVGWACTTLCLASCSSAFSHGASTVACDDMQPKHIQAQPQHQDSHHITIHTHRTSYAPGDKIPVTVRSSRDFMGFLLQARRVSDHQIAGTFVLIPPHSKLMTCFQEADAVTHSDKSLKRNLSFVWKAPAQPVGDIKFLLSVVQSYFVYWARIESSVVSQQTHSSAHSDDRMEPRLLMPNLHQRLGDVEGAAPAPRTPITLPQQHTHVFAVALPGAAEEDNLDPVPASIWVTKFPGDAETLSQPSSHTATEGSINQQPSGDSNPTLEPSLEVHRLERLVALKRVSSESFASSLSTHHRTQDDPSFDSLETCLSSDGGEQVRTHHHHHH

SEQ ID NO: 33 - DNA, 495 bp, Artificial Sequence
  INSP171-SV1 his tag Full length polynucleotide
atgaggatgcaggctgccctcgtgggctgggcttgtaccaccctctgcctggcttcctgctcatctgccttttcccatggtgccagcacggtggcctgtgatgacatgcagcccaagcacatccaagcccagcctcagcaccaggacagccaccacatcaccatccacacccacaggacttcctatgcaccaggtgacaagattccagtgacagtgagaagcagtcgtgatttcatgggatttctccttcaggctcgaagagtgtccgatcatcaaatcgctggcactttcgttctcattcctcctcattccaaactgatgacttgttttcaagaggctgatgcagtcacccactctgacaagtccctgaagagaaacctgtcattcgtgtggaaggccccagcccagcctgtgggggacattaagttcctacacatagcagcgcccattctgacgaccgcatggagcccagattgccaccatcaccatcaccat

SEQ ID NO: 34 - protein, 165 aa, Artificial Sequence
  INSP171-SV1 his tag Full length polypeptide
MRMQAALVGWACTTLCLASCSSAFSHGASTVACDDMQPKHIQAQPQHQDSHHITIHTHRTSYAPGDKIPVTVRSSRDFMGFLLQARRVSDHQIAGTFVLIPPHSKLMTCFQEADAVTHSDKSLKRNLSFVWKAPAQPVGDIKFLHIAAPILTTAWSPDCHHHHHH

SEQ ID NO: 35 - DNA, 312 bp, Artificial Sequence
  INSP171-SV2 his tag Full length polynucleotide
atgaggatgcaggctgccctcgtgggctgggcttgtaccaccctctgcctggcttcctgctcatctgccttttcccatggtgccagcacggtggcctgtgatgacatgcagcccaagcacatccaagcccagcctcagcaccaggacagccaccacatcaccatccacacccacaggacttcctatgcaccaggtgacaagattccaggctcgaagagtgtccgatcatcaaatcgctggcactttcgttctcattcctcctcattccaaactgatgacttgttttcaagaggccaccatcaccatcaccat

SEQ ID NO: 36 - protein, 104 aa, Artificial Sequence
  INSP171-SV2 his tag Full length polypeptide
MRMQAALVGWACTTLCLASCSSAFSHGASTVACDDMQPKHIQAQPQHQDSHHITIHTHRTSYAPGDKIPGSKSVRSSNRWHFRSHSSSFQTDDLFSRGHHHHHH

SEQ ID NO: 37 - DNA, 285 bp, Artificial Sequence
  INSP171-SV3 his tag Full length polynucleotide
atgaggatgcaggctgccctcgtgggctgggcttgtaccaccctctgcctggcttcctgctcatctgccttttcccatggtgccagcacggtggcctgtgatgacatgcagcccaagcacatccaagcccagcctcagcaccaggacagccaccacatcaccatccacacccacaggacttcctatgcaccaggtgacaagattccaggtatcctgagggaaacacaattggattgtaactggaaccaagacagcatatgtaataaacaccatcaccatcaccat

SEQ ID NO: 38 - protein, 95 aa, Artificial Sequence
  INSP171-SV3 his tag Full length polypeptide
MRMQAALVGWACTTLCLASCSSAFSHGASTVACDDMQPKHIQAQPQHQDSHHITIHTHRTSYAPGDKIPGILRETQLDCNWNQDSICNKHHHHHH

SEQ ID NO: 39 - DNA, 228 bp, Artificial Sequence
  INSP171-SV4 his tag Full length polynucleotide
atgaggatgcaggctgccctcgtgggctgggcttgtaccaccctctgcctggcttcctgctcatctgccttttcccatggtgccagcacggtggcctgtgatgacatgcagcccaagcacatccaagcccagcctcagcaccaggacagccaccacatcaccatccacacccacaggacttcctatgcaccaggtgacaagattccagttcaccatcaccatcaccat

SEQ ID NO: 40 - protein, 76 aa, Artificial Sequence
  INSP171-SV4 his tag Full length polypeptide
MRMQAALVGWACTTLCLASCSSAFSHGASTVACDDMQPKHIQAQPQHQDSHHITIHTHRTSYAPGDKIPVHHHHHH

SEQ ID NO: 41 - protein, 70 aa, Homo sapiens; identical to SEQ ID NO: 30

SEQ ID NO: 42 - protein, 68 aa, Homo sapiens
MQAALVGWACTTLCLASCSSAFSHGASTVACDDMQPKHIQAQPQHQDSHHITIHTHRTSYAPGDKIPV

SEQ ID NO: 43 - protein, 13 aa, Artificial Sequence
  Fusion protein linker sequence
EFGAGLVLGGQFM

SEQ ID NO: 44 - DNA, 20 bp, Artificial Sequence
  PCR primer
gtgggctgggcttgtaccac

SEQ ID NO: 45 - DNA, 21 bp, Artificial Sequence
  PCR primer
tcacccccatctgaggacagg

SEQ ID NO: 46 - DNA, 56 bp, Artificial Sequence
  PCR primer
gcaggcttcgccaccatgaggatgcaggctgccctcgtgggctgggcttgtaccac

SEQ ID NO: 47 - DNA, 48 bp, Artificial Sequence
  PCR primer
tgatggtgatggtgggttcttacctgttcacccccatctgaggacagg

SEQ ID NO: 48 - DNA, 35 bp, Artificial Sequence
  PCR primer
tgatggtgatggtggcaatctgggctccatgcggt

SEQ ID NO: 49 - DNA, 35 bp, Artificial Sequence
  PCR primer
tgatggtgatggtggcctcttgaaaacaagtcatc

SEQ ID NO: 50 - DNA, 35 bp, Artificial Sequence
  PCR primer
tgatggtgatggtgtttattacatatgctgtcttg

SEQ ID NO: 51 - DNA, 35 bp, Artificial Sequence
  PCR primer
tgatggtgatggtgaactggaatcttgtcacctgg

SEQ ID NO: 52 - DNA, 31 bp, Artificial Sequence
  PCR primer
ggtgccagcacggtggcctgtgatgacatgc

SEQ ID NO: 53 - DNA, 33 bp, Artificial Sequence
  PCR primer
catgtcatcacaggccaccgtgctggcaccatg

SEQ ID NO: 54 - DNA, 20 bp, Artificial Sequence
  PCR primer
gtgggctgggcttgtaccac

SEQ ID NO: 55 - DNA, 20 bp, Artificial Sequence
  PCR primer
atgccaaaccttcaccagag

SEQ ID NO: 56 - DNA, 20 bp, Artificial Sequence
  PCR primer
aaagctgggatcatcctgag

SEQ ID NO: 57 - DNA, 37 bp, Artificial Sequence
  PCR primer
ggggacaagtttgtacaaaaaagcaggcttcgccacc

SEQ ID NO: 58 - DNA, 51 bp, Artificial Sequence
  PCR primer
ggggaccactttgtacaagaaagctgggtttcaatggtgatggtgatggtg

SEQ ID NO: 59 - DNA, 20 bp, Artificial Sequence
  PCR primer
gccagcttggcacttgatgt

SEQ ID NO: 60 - DNA, 20 bp, Artificial Sequence
  PCR primer
gatggaggtggacgtgtcag

SEQ ID NO: 61 - DNA, 18 bp, Artificial Sequence
  PCR primer
tgtaaaacgacggccagt

SEQ ID NO: 62 - DNA, 18 bp, Artificial Sequence
  PCR primer
caggaaacagctatgacc

SEQ ID NO: 63 - DNA, 19 bp, Artificial Sequence
  PCR primer
taatacgactcactatagg

SEQ ID NO: 64 - DNA, 18 bp, Artificial Sequence
  PCR primer
attaaccctcactaaagg

SEQ ID NO: 65 - DNA, 1267 bp, Homo sapiens
  exon (142)..(1123)
tgggaggctgcactaaaagcatctcaggtacagaggcagtgtggggttcaggttagtcacatcccgagaagactttatgtcgttttcctaaataacgtctggactgaagctgctgtttgtcatgatacaggagagggcaggatgaggatgcaggctgccctcgtgggctgggcttgtaccaccctctgcctggcttcctgctcatctgccttttcccatggtgccagcacggtggcctgtgatgacatgcagcccaagcacatccaagcccagcctcagcaccaggacagccaccacatcaccatccacacccacaggacttcctatgcaccaggtgacaagattccagtgacagtgagaagcagtcgtgatttcatgggatttctccttcaggctcgaagagtgtccgatcatcaaatcgctggcactttcgttctcattcctcctcattccaaactgatgacttgttttcaagaggctgatgcagtcacccactctgacaagtccctgaagagaaacctgtcattcgtgtggaaggccccagcccagcctgtgggggacattaagttccttttatcagtagtccagtcatattttgtttactgggcaaggattgaatcatctgttgtgtctcaacagacacatagcagcgcccattctgacgaccgcatggagcccagattgctgatgccaaaccttcaccagaggctgggcgatgttgaaggagctgctccagctcccaggacccccatcactcttccacagcagcacacacatgtctttgctgttgcccttcctggagctgcagaggaggacaacctagatcctgttcctgccagtatttgggtgacaaagtttcctggggatgcagagactctgtcccaaccatcttcacacacagccactgaaggcagcatcaaccagcaacccagcggggacagcaatcccaccctagagccgtccctggaggtccacaggctggagaggctcgtggccctcaagagagtctcctcagagagctttgcttccagccttagcacccatcacagaactcaggatgatcccagctttgattcactggaaacttgcctgtcctcagatgggggtgaacaggtaagaacctgatgggaggctgcactaaaagcatctcaggtacagaggcagtgtggggttcaggttagtcacatcccgagaagactttatgtcgttttcctaaataacgtctggactgaagctgctgtttgtcatgatacaggagagggcagga

SEQ ID NO: 66 - protein, 126 aa, Homo sapiens
ACDDMQPKHIQAQPQHQDSHHITIHTHRTSYAPGDKIPVTVRSSRDFMGFLLQARRVSDHQIAGTFVLIPPHSKLMTCFQEADAVTHSDKSLKRNLSFVWKAPAQPVGDIKFLLSVVQSYFVYWAR

SEQ ID NO: 67 - protein, 127 aa, Homo sapiens
ACDDMQPKHIQAQPQHQDSHHITIHTHRTSYAPGDKIPVTVRSSRDFMGFLLQARRVSDHQIAGTFVLIPPHSKLMTCFQEADAVTHSDKSLKRNLSFVWKAPAQPVGDIKFLHIAAPILTTAWSPD

SEQ ID NO: 68 - protein, 125 aa, Mus musculus
CGGMIPQHNHSPQSEPIHQITVSQTTFKPEDQIEVTLSGPPFRGFLLEARDAENLSGPPIGSFTLIDSEESQLLTCTDVQGLAVSHTRSSKKTEIKVYWDAPSPAPDHIRFLATVVQKFKIYWVK

SEQ ID NO: 69 - protein, 131 aa, Homo sapiens
YCSRILRAQGTRREGYTEFSLRVEGDPDFYKPGTSYRVTLSAAPPSYFRGFTLIALRENREGDKEEDHAGTFQIIDEEETQFMSNCPVAVTESTPRRRTRIQVFWIAPPAGTGCVILKASIVQKRIIYFQD

SEQ ID NO: 70 - DNA, 947 bp, Homo sapiens
  exon (1)..(945)
gtgggctgggcttgtaccaccctctgcctggcttcctgctcatctgccttttcccatggtgccagcacggtggcctgtgatgacatgcagcccaagcacatccaagcccagcctcagcaccaggacagccaccacatcaccatccacacccacaggacttcctatgcaccaggtgacaagattccagtgacagtgagaagcagtcgtgatttcatgggatttctccttcaggctcgaagagtgtccgatcatcaaatcgctggcactttcgttctcattcctcctcattccaaactgatgacttgttttcaagaggctgatgcagtcacccactctgacaagtccctgaagagaaacctgtcattcgtgtggaaggccccagcccagcctgtgggggacattaagttccttttatcagtagtccagtcatattttgtttactgggcaaggattgaatcatctgttgtgtctcaacagacacatagcagcgcccattctgacgaccgcatggagcccagattgctgatgccaaaccttcaccagaggctgggcgatgttgaaggagctgctccagctcccaggacccccatcactcttccacagcagcacacacatgtctttgctgttgcccttcctggagctgcagaggaggacaacctagatcctgttcctgccagtatttgggtgacaaagtttcctggggatgcagagactctgtcccaaccatcttcacacacagccactgaaggcagcatcaaccagcaacccagcggggacagcaatcccaccctagagccgtccctggaggtccacaggctggagaggctcgtggccctcaagagagtctcctcagagagctttgcttccagccttagcacccatcacagaactcaggatgatcccagctttgattcactggaaacttgcctgtcctcagatgggggtga

SEQ ID NO: 71 - DNA, 880 bp, Homo sapiens
  exon (1)..(456)
gtgggctgggcttgtaccaccctctgcctggcttcctgctcatctgccttttcccatggtgccagcacggtggcctgtgatgacatgcagcccaagcacatccaagcccagcctcagcaccaggacagccaccacatcaccatccacacccacaggacttcctatgcaccaggtgacaagattccagtgacagtgagaagcagtcgtgatttcatgggatttctccttcaggctcgaagagtgtccgatcatcaaatcgctggcactttcgttctcattcctcctcattccaaactgatgacttgttttcaagaggctgatgcagtcacccactctgacaagtccctgaagagaaacctgtcattcgtgtggaaggccccagcccagcctgtgggggacattaagttcctacacatagcagcgcccattctgacgaccgcatggagcccagattgctgatgccaaaccttcaccagaggctgggcgatgttgaaggagctgctccagctcccaggacccccatcactcttccacagcagcacacacatgtctttgctgttgcccttcctggagctgcagaggaggacaacctagatcctgttcctgccagtatttgggtgacaaagtttcctggggatgcagagactctgtcccaaccatcttcacacacagccactgaaggcagcatcaaccagcaacccagcggggacagcaatcccaccctagagccgtccctggaggtccacaggctggagaggctcgtggccctcaagagagtctcctcagagagctttgcttccagccttagcacccatcacagaactcaggatgatcccagctttgattcactggaaacttgcctgtcctcagatgggggtga

SEQ ID NO: 72 - DNA, 836 bp, Homo sapiens
  exon (1)..(273)
gtgggctgggcttgtaccaccctctgcctggcttcctgctcatctgccttttcccatggtgccagcacggtgacctgtgatgacatgcagcccaagcacatccaagcccagcctcagcaccaggacagccaccacatcaccatccacacccacaggacttcctatgcaccaggtgacaagattccaggctcgaagagtgtccgatcatcaaatcgctggcactttcgttctcattcctcctcattccaaactgatgacttgttttcaagaggctgatgcagtcacccactctgacaagtccctgaagagaaacctgtcattcgtgtggaaggccccagcccagcctgtgggggacattaagttcctacacatagcagcgcccattctgacgaccgcatggagcccagattgctgatgccaaaccttcaccagaggctgggcgatgttgaaggagctgctccagctcccaggacccccatcactcttccacagcagcacacacatgtctttgctgttgcccttcctggagctgcagaggaggacaacctagatcctgttcctgccagtatttgggtgacaaagtttcctggggatgcagagactctgtcccaaccatcttcacacacagccactgaaggcagcatcaaccagcaacccagcggggacagcaatcccaccctagagccgtccctggaggtccacaggctggagaggctcgtggccctcaagagagtctcctcagagagctttgcttccagccttagcacccatcacagaactcaggatgatcccagctttgattcactggaaacttgcctgtcctcagatgggggtga

SEQ ID NO: 73 - DNA, 993 bp, Homo sapiens
  exon (1)..(246)
gtgggctgggcttgtaccaccctctgcctggcttcctgctcatctgccttttcccatggtgccagcacggtggcctgtgatgacatgcagcccaagcacatccaagcccagcctcagcaccaggacagccaccacatcaccatccacacccacaggacttcctatgcaccaggtgacaagattccaggtatcctgagggaaacacaattggattgtaactggaaccaagacagcatatgtaataaatgacttgacagagtgcctcatggattcaatgagagtcagttattactgacttacgttgcaagtatactgttttcttgccgggatctgtgagctccttgaaggctcgaagagtgtccgatcatcaaatcgctggcactttcgttctcattcctcctcattccaaactgatgacttgttttcaagaggctgatgcagtcacccactctgacaagtccctgaagagaaacctgtcattcgtgtggaaggccccagcccagcctgtgggggacattaagttcctacacatagcagcgcccattctgacgaccgcatggagcccagattgctgatgccaaaccttcaccagaggctgggcgatgttgaaggagctgctccagctcccaggacccccatcactcttccacagcagcacacacatgtctttgctgttgcccttcctggagctgcagaggaggacaacctagatcctgttcctgccagtatttgggtgacaaagtttcctggggatgcagagactctgtcccaaccatcttcacacacagccactgaaggcagcatcaaccagcaacccagcggggacagcaatcccaccctagagccgtccctggaggtccacaggctggagaggctcgtggccctcaagagagtctcctcagagagctttgcttccagccttagcacccatcacagaactcaggatgatcccagctttgattcactggaaacttgcctgtcctcagatggggg

SEQ ID NO: 74 - DNA, 829 bp, Homo sapiens
  exon (1)..(189)
gtgggctgggcttgtaccaccctctgcctggcttcctgctcatctgccttttcccatggtgccagcacggtggcctgtgatgacatgcagcccaagcacatccaagcccagcctcagcaccaggacagccaccacatcaccatccacacccacaggacttcctatgcaccaggtgacaagattccagtttaggtgcctataaaagcagggaaagaacgtgagcgcaggtggtttatgtgagagatcatcccaggaaacacagaggctgatgcagtcacccactctgacaagtccctgaagagaaacctgtcattcgtgtggaaggccccagcccagcctgtgggggacattaagttcctacacatagcagcgcccattctgacgaccgcatggagcccagattgctgatgccaaaccttcaccagaggctgggcgatgttgaaggagctgctccagctcccaggacccccatcactcttccacagcagcacacacatgtctttgctgttgcccttcctggagctgcagaggaggacaacctagatcctgttcctgccagtatttgggtgacaaagtttcctggggatgcagagactctgtcccaaccatcttcacacacagccactgaaggcagcatcaaccagcaacccagcggggacagcaatcccaccctagagccgtccctggaggtccacaggctggagaggctcgtggccctcaagagagtctcctcagagagctttgcttccagccttagcacccatcacagaactcaggatgatcccagctttgattcactggaaacttgcctgtcctcagatgggggtga